(19) United States
(12) Patent Application Publication
(10) Pub. No.: US 2022/0193246 A1
(43) Pub. Date: Jun. 23, 2022

(54) COMPOSITIONS FOR TREATING MUSCULAR DYSTROPHY

(71) Applicant: Sarepta Therapeutics, Inc., Cambridge, MA (US)

(72) Inventors: Sanjay RAKHADE, Cambridge, MA (US); Jay CHARLESTON, Cambridge, MA (US)

(73) Assignee: Sarepta Therapeutics, Inc., Cambridge, MA (US)

(21) Appl. No.: 17/603,229

(22) PCT Filed: Apr. 16, 2020
(86) PCT No.: PCT/US2020/028433

§ 371 Date: Oct. 12, 2021

Related U.S. Application Data
(60) Provisional application No. 62/835,836, filed on Apr. 18, 2019.

(51) Int. Cl. A61K 47/64

(52) U.S. Cl. CPC A61K 47/645

(57) ABSTRACT

Methods for treating Duchenne muscular dystrophy using conjugates of cell penetrating peptides to antisense oligonucleotide that induce exon skipping in the human dystrophin gene. In one embodiment, the cell penetrating peptide is an arginine-rich peptide comprising six contiguous arginine residues and the antisense oligonucleotide is eteplirsen, golodirsen or casimersen.

### CROSS-REFERENCE TO RELATED APPLICATIONS

This application claims the benefit of U.S. Provisional Application No. 62/835,836, filed on Apr. 18, 2019. The entire teachings of the above-referenced application are incorporated by reference in their entirety.

### SEQUENCE LISTING

The instant application contains a Sequence Listing which has been submitted electronically in ASCII format and is hereby incorporated by reference in its entirety. Said ASCII copy, created on Mar. 19, 2020, is named 8164_51_WO00_SL.txt and is 51,048 bytes in size.

### FIELD OF THE INVENTION

The present invention relates to improved methods for treating diseases or disorders amenable to antisense oligonucleotide therapy comprising an effective amount of an antisense oligonucleotide or an antisense oligonucleotide conjugate, or a pharmaceutically acceptable salt thereof, e.g., muscular dystrophy, in a human patient.

### BACKGROUND OF THE INVENTION

Antisense technologies are being developed using a range of chemistries to affect gene expression at a variety of different levels (transcription, splicing, stability, translation). Much of that research has focused on the use of antisense compounds to correct or compensate for abnormal or disease-associated genes in a wide range of indications. Antisense molecules are able to inhibit gene expression with specificity, and because of this, many research efforts concerning oligomers as modulators of gene expression have focused on inhibiting the expression of targeted genes or the function of cis-acting elements. The antisense oligomers are typically directed against RNA, either the sense strand (e.g., mRNA), or minus-strand in the case of some viral RNA targets. To achieve a desired effect of specific gene down-regulation, the oligomers generally either promote the decay of the targeted mRNA, block translation of the mRNA or block the function of cis-acting RNA elements, thereby effectively preventing either de novo synthesis of the target protein or replication of the viral RNA.

However, such techniques are not useful where the object is to up-regulate production of the native protein or compensate for mutations that induce premature termination of translation, such as nonsense or frame-shifting mutations. In these cases, the defective gene transcript should not be subjected to targeted degradation or steric inhibition, so the antisense oligomer chemistry should not promote target mRNA decay or block translation.

In a variety of genetic diseases, the effects of mutations on the eventual expression of a gene can be modulated through a process of targeted exon skipping during the splicing process. The splicing process is directed by complex multi-component machinery that brings adjacent exon-intron junctions in pre-mRNA into close proximity and performs cleavage of phosphodiester bonds at the ends of the introns with their subsequent reformation between exons that are to be spliced together. This complex and highly precise process is mediated by sequence motifs in the pre-mRNA that are relatively short, semi-conserved RNA segments to which various nuclear splicing factors that are then involved in the splicing reactions bind. By changing the way the splicing machinery reads or recognizes the motifs involved in pre-mRNA processing, it is possible to create differentially spliced mRNA molecules. It has now been recognized that the majority of human genes are alternatively spliced during normal gene expression, although the mechanisms involved have not been identified. Bennett et al. (U.S. Pat. No. 6,210,892) describe antisense modulation of wild-type cellular mRNA processing using antisense oligomer analogs that do not induce RNAse H-mediated cleavage of the target RNA. This finds utility in being able to generate alternatively spliced mRNAs that lack specific exons.

In cases where a normally functional protein is prematurely terminated because of mutations therein, a means for restoring some functional protein production through antisense technology has been shown to be possible through intervention during the splicing processes, and that if exons associated with disease-causing mutations can be specifically deleted from some genes, a shortened protein product can sometimes be produced that has similar biological properties of the native protein or has sufficient biological activity to ameliorate the disease caused by mutations associated with the exon. Kole et al. (U.S. Pat. Nos. 5,627,274; 5,916,808; 5,976,879; and 5,665,593) disclose methods of combating aberrant splicing using modified antisense oligomer analogs that do not promote decay of the targeted pre-mRNA. Bennett et al. (U.S. Pat. No. 6,210,892) describe antisense modulation of wild-type cellular mRNA processing also using antisense oligomer analogs that do not induce RNAse H-mediated cleavage of the target RNA.

The process of targeted exon skipping is likely to be particularly useful in long genes where there are many exons and introns, where there is redundancy in the genetic constitution of the exons or where a protein is able to function without one or more particular exons. Efforts to redirect gene processing for the treatment of genetic diseases associated with truncations caused by mutations in various genes have focused on the use of antisense oligomers that either: (1) fully or partially overlap with the elements involved in the splicing process; or (2) bind to the pre-mRNA at a position sufficiently close to the element to disrupt the binding and function of the splicing factors that would normally mediate a particular splicing reaction which occurs at that element.

Duchenne muscular dystrophy (DMD) is caused by a defect in the expression of the protein dystrophin. The gene encoding the protein contains 79 exons spread out over more than 2 million nucleotides of DNA. Any exonic mutation that changes the reading frame of the exon, or introduces a stop codon, or is characterized by removal of an entire out of frame exon or exons, or duplications of one or more exons, has the potential to disrupt production of functional dystrophin, resulting in DMD.

A less severe form of muscular dystrophy, Becker muscular dystrophy (BMD) has been found to arise where a mutation, typically a deletion of one or more exons, results in a correct reading frame along the entire dystrophin transcript, such that translation of mRNA into protein is not prematurely terminated. If the joining of the upstream and downstream exons in the processing of a mutated dystrophin pre-mRNA maintains the correct reading frame of the gene, the result is an mRNA coding for a protein with a short internal deletion that retains some activity, resulting in a Becker phenotype.

For many years it has been known that deletions of an exon or exons which do not alter the reading frame of a dystrophin protein would give rise to a BMD phenotype, whereas an exon deletion that causes a frame-shift will give rise to DMD. In general, dystrophin mutations including point mutations and exon deletions that change the reading frame and thus interrupt proper protein translation result in DMD. It should also be noted that some BMD and DMD patients have exon deletions covering multiple exons.

Although at least one product has been approved for the treatment of DMD, there remains a need for improved compositions and methods for treating muscular dystrophy, such as DMD and BMD in patients.

### BRIEF DESCRIPTION OF FIGURE

FIG. 1 shows exon skipping in muscle biopsies over 28 days following a single IV infusion of vehicle or 30 mg or 60 mg of PPMO #1 (as a 6HCl salt form) to cynomolgus monkeys.

### SUMMARY OF THE INVENTION

Methods of treating a human patient having a disease or disorder amenable to antisense oligonucleotide therapy comprising an effective amount of an antisense oligonucleotide or an antisense oligonucleotide conjugate, or a pharmaceutically acceptable salt thereof, are provided herein. In some aspects, the nucleobases of the antisense oligonucleotide are linked to morpholino ring structures. In some aspects, the morpholino subunits are joined by phosphorous-containing intersubunit linkages joining a morpholino nitrogen of one subunit to a 5′ exocyclic carbon of an adjacent morpholino subunit

In certain embodiments, the antisense oligonucleotide conjugate comprises an antisense oligonucleotide conjugated to one or more cell-penetrating peptides (referred to herein as “CPP”). In certain aspects, the nucleobases of the antisense oligonucleotide conjugate are linked to morpholino ring structures. In some aspects, the morpholino subunits are joined by phosphorous-containing intersubunit linkages joining a morpholino nitrogen of one subunit to a 5′ exocyclic carbon of an adjacent morpholino subunit.

In some embodiments, the CPP is an arginine-rich peptide. The term “arginine-rich” refers to a CPP having at least 2, and preferably 2, 3, 4, 5, 6, 7, or 8 arginine residues, each optionally separated by one or more uncharged, hydrophobic residues, and optionally containing about 6-14 amino acid residues. In some aspects, the arginine-rich peptide is selected from the group consisting of —(RXR)₄—R^{a} (SEQ ID NO: 52), —R—(FFR)₃—R^{a} (SEQ ID NO: 53), —B—X—(RXR)₄—R^{a} (SEQ ID NO: 54), —B—X—R—(FFR)₃—R^{a} (SEQ ID NO: 55), -GLY-R—(FFR)₃—R^{a} (SEQ ID NO: 56), -GLY-R₅—R^{a} (SEQ ID NO: 59), —R₅—R^{a} (SEQ ID NO: 60), -GLY-R₆—R^{a} (SEQ ID NO: 57) and —R₆—R^{a} (SEQ ID NO: 58), wherein R^{a} is selected from H, acyl, benzoyl, and stearoyl, and wherein R is arginine, X is 6-aminohexanoic acid, B is β-alanine, F is phenylalanine and GLY (or G) is glycine.

Methods of treating a human patient having Duchenne muscular dystrophy, the methods comprising administering to the human patient a therapeutically effective amount of an antisense oligonucleotide or an antisense oligonucleotide conjugate that comprises a cell penetrating peptide and an oligonucleotide, or a pharmaceutically acceptable salt thereof, once every four weeks are provided herein, wherein the antisense oligonucleotide or antisense oligonucleotide conjugate is capable of binding a selected target to induce exon skipping in the human dystrophin gene. In some aspects, the antisense oligonucleotide or antisense oligonucleotide conjugate induces skipping of exon 44, exon 45, exon 50, exon 51, exon 52, or exon 53 target region of the dystrophin pre-mRNA. In certain aspects, the human patient is administered an antisense oligonucleotide conjugate, or pharmaceutically acceptable salt thereof.

In some aspects, the human patient is administered the antisense oligonucleotide or antisense oligonucleotide conjugate, or pharmaceutically acceptable salt thereof, at least six months, at least one year, at least two years, at least three years, at least four years, or at least five years.

In some aspects, the methods comprise administering an antisense oligonucleotide conjugate, or a pharmaceutically acceptable salt thereof, that comprises a cell penetrating peptide that is an arginine-rich peptide attached to the oligonucleotide. In some aspects, the antisense oligonucleotide conjugate comprises a cell penetrating peptide that is an arginine-rich peptide that is -GLY-R₅—R^{a} (SEQ ID NO: 59), —R₅—R^{a} (SEQ ID NO: 60), -GLY-R₆—R^{a} (SEQ ID NO: 57) or —R₆—R^{a} (SEQ ID NO: 58), wherein R is arginine and R^{a} is hydrogen or an acyl group.

In some embodiments, the antisense oligonucleotide or antisense oligonucleotide conjugate, is provided in a pharmaceutical composition formed by dissolving 0.005 mg/kg to about 300 mg/kg of the antisense oligonucleotide conjugate, or pharmaceutically acceptable salt thereof, in an aqueous carrier solution.

In some embodiments, the therapeutically effective amount of the antisense oligonucleotide or antisense oligonucleotide conjugate, or pharmaceutically acceptable salt thereof, is in an amount from about 0.005 mg/kg to about 300 mg/kg. In certain aspects, the therapeutically effective amount of the antisense oligonucleotide or antisense oligonucleotide conjugate, or pharmaceutically acceptable salt thereof, is at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg. In certain aspects, the therapeutically effective amount of the antisense oligonucleotide or antisense oligonucleotide conjugate, or pharmaceutically acceptable salt thereof, is about 0.005 mg/kg to about 200 mg/kg, about 0.1 mg/kg to about 100 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, or about 80 mg/kg to about 300 mg/kg. In certain aspects, the therapeutically effective amount of the antisense oligonucleotide or antisense oligonucleotide conjugate, or pharmaceutically acceptable salt thereof, is about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg.

In some embodiments, the antisense oligonucleotide or antisense oligonucleotide conjugate, or pharmaceutically acceptable salt thereof, is administered intravenously.

Methods of treating a human patient having Duchenne muscular dystrophy, the method comprising administering to the human patient a pharmaceutical composition comprising a therapeutically effective amount of an antisense oligonucleotide or an antisense oligonucleotide conjugate, or pharmaceutically acceptable salt thereof, wherein the antisense oligonucleotide conjugate comprises a cell penetrating peptide and an oligonucleotide that induces skipping of an exon 44, exon 45, exon 50, exon 51, exon 52, or exon 53 target region of the dystrophin pre-mRNA, or a pharmaceutically acceptable salt thereof. In certain aspects, the human patient is administered an antisense oligonucleotide conjugate, or pharmaceutically acceptable salt thereof.

In certain embodiments, the antisense oligonucleotide conjugate comprises a cell penetrating peptide that is an arginine-rich peptide. In some aspects, the antisense oligonucleotide conjugate comprises a cell penetrating peptide that is an arginine-rich peptide selected from the group consisting of —(RXR)₄—R^{a} (SEQ ID NO: 52), —R—(FFR)₃—R^{a} (SEQ ID NO: 53), —B—X—(RXR)₄—R^{a} (SEQ ID NO: 54), —B—X—R—(FFR)₃—R^{a} (SEQ ID NO: 55), -GLY-R—(FFR)₃—R^{a} (SEQ ID NO: 56), -GLY-R₅—R^{a}, —R₅—R^{a}, (SEQ ID NO: 59), -GLY-R₆—R^{a} (SEQ ID NO: 57), and —R₆—R^{a} (SEQ ID NO: 58), wherein R^{a} is selected from H, acyl, benzoyl, and stearoyl, and wherein R is arginine, X is 6-aminohexanoic acid. B is β-alanine, F is phenylalanine and GLY (or G) is glycine. In some aspects, the antisense oligonucleotide conjugate comprises a cell penetrating peptide that is an arginine-rich peptide that is —R₅—R^{a} (SEQ ID NO: 60) or —R₆—R^{a} (SEQ ID NO: 58), wherein R^{a} is an acyl group. In some aspects, the antisense oligonucleotide conjugate comprises a cell penetrating peptide that is an arginine-rich peptide that is —R₆—R^{a} (SEQ ID NO: 58), wherein R^{a} is an acyl group. In some aspects, the arginine-rich peptide is -GLY-R₅—R^{a} (SEQ ID NO: 59) or -GLY-R₆—R^{a} (SEQ ID NO: 57), wherein R^{a} is an acyl group. In some aspects, the arginine-rich peptide is -GLY-R₆—R^{a}, (SEQ ID NO: 57), wherein R^{a} is an acyl group. In some aspects, the antisense oligonucleotide conjugate comprises a cell penetrating peptide that is an arginine-rich peptide that is —R₅—R^{a} (SEQ ID NO: 60) or —R₆—R^{a} (SEQ ID NO: 58), wherein R^{a} is H. In some aspects, the antisense oligonucleotide conjugate comprises a cell penetrating peptide that is an arginine-rich peptide that is —R₆—R^{a} (SEQ ID NO: 58), wherein R^{a} H. In some aspects, the arginine-rich peptide is -GLY-R₅—R^{a} (SEQ ID NO: 59) or -GLY-R₆—R^{a} (SEQ ID NO: 57), wherein R^{a} is H. In some aspects, the arginine-rich peptide is -GLY-R₆—R^{a} (SEQ ID NO: 57), wherein R^{a} is H.

In some embodiments, the target region for the antisense oligonucleotide or antisense oligonucleotide conjugate is designated as an annealing site, and wherein the base sequence and annealing site are selected from one of the following:

| | | SEQ |
|---|---|---|
| | Base Sequence | ID |
| Annealing Site | [5′ to 3′] | NO. |
| H51A(+66+95) | CTC CAA CAT CAA GGA | 1 |
| | AGA TGG CAT TTC TAG | |
| H51A(+74+97) | ACC TCC AAC ATC AAG | 2 |
| | GAA GAT GGC | |
| H51A(+70+99) | GTA CCT CCA ACA TCA | 3 |
| | AGG AAG ATG GCA TTT | |
| H51A(+72+99) | GTA CCT CCA ACA TCA | 4 |
| | AGG AAG ATG GCA T | |
| H51A(+68+87) | TCA AGG AAG ATG GCA | 5 |
| | TTT CT | |
| H51A(+68+87) | UCA AGG AmAGm AmUGm | 6 |
| | GmCA UUU CU | |
| H53A(+36+60) | GTT GCC TCC GGT TCT | 7 |
| | GAA GGT GTT C | |
| H53A(+36+60) | GTT G5mC5mC T5mC5mC | 8 |
| | GGT T5mC T GAA GGT | |
| | GTT 5mC | |
| H53A(+36+56) | CCT CCG GTT CTG AAG | 9 |
| | GTG TTC | |
| H53A(+23+47) | CTG AAG GTG TTC TTG | 10 |
| | TAC TTC ATC C | |
| H53A(+32+56) | CCT CCG GTT CTG AAG | 11 |
| | GTG TTC TTG T | |
| H53A(+33+60) | GTT GCC TCC GGT TCT | 12 |
| | GAA GGT GTT CTT G | |
| H53A(+30+59) | TTG CCT CCG GTT CTG | 13 |
| | AAG GTG TTC TTG TAC | |
| H53A(+39+62) | CTG TTG CCT CCG GTT | 14 |
| | CTG AAG GTG | |
| H53A(+36+69) | CAT TCA ACT GTT GCC | 15 |
| | TCC GGT TCT GAA GGT | |
| | G | |
| H53A(+45+62) | CTG TTG CCT CCG GTT | 16 |
| | CTG | |
| H45A(−03+19) | CAA TGC CAT CCT GGA | 17 |
| | GTT CCT G | |
| H45A(−09+25) | GCT GCC CAA TGC CAT | 18 |
| | CCT GGA GTT CCT GTA | |
| | AGA T | |
| H45A(−03+25) | GCT GCC CAA TGC CAT | 19 |
| | CCT GGA GTT CCT G | |
| H45A(−06+25) | GCT GCC CAA TGC CAT | 20 |
| | CCT GGA GTT CCT GTA | |
| | A | |
| H45A(−12+19) | CAA TGC CAT CCT GGA | 21 |
| | GTT CCT GTA AGA TAC | |
| | C | |
| H45A(−09+19) | CAA TGC CAT CCT GGA | 22 |
| | GTT CCT GTA AGA T | |
| H45A(−12+16) | TGC CAT CCT GGA GTT | 23 |
| | CCT GTA AGA TAC C | |
| H45A(−l4+25) | GCT GCC CAA TGC CAT | 24 |
| | CCT GGA GTT CCT GTA | |
| | AGA TAC CAA | |
| H45A(−08+19) | CAA TGC CAT CCT GGA | 25 |
| | GTT CCT GTA AGA | |
| HM45A(−07+25) | GCT GCC CAA TGC CAT | 26 |
| | CCT GGA GTT CCT GTA | |
| | AG | |
| H45A(−12+22) | GCC CAA TGC CAT CCT | 27 |
| | GGA GTT CCT GTA AGA | |
| | TAC C | |
| H45A(−09+22) | GCC CAA TGC CAT CCT | 28 |
| | GGA GTT CCT GTA AGA | |
| | T | |
| H45A(−09+30) | TTG CCG CTG CCC AAT | 29 |
| | GCC ATC CTG GAG TTC | |
| | CTG TAA GAT | |
| H45A(−06+22) | GCC CAA TGC CAT CCT | 30 |
| | GGA GTT CCT GTA A | |
| H45A(−06+28) | GCC GCT GCC CAA TGC | 31 |
| | CAT CCT GGA GTT CCT | |
| | GTA A | |
| H45A(−03+22) | GCC CAA TGC CAT CCT | 32 |
| | GGA GTT CCT G | |
| H45A(−03+28) | GCC GCT GCC CAA TGC | 33 |
| | CAT CCT GGA GTT CCT | |
| | G | |
| H45A(+9+26) | m5C-G-m5C-T-G-C-m5C- | 34 |
| | m5C-A-A-T-G-m5C-m5C- | |
| | A-U-m5C-m5C | |
| H44A(−10+15) | GAT CTG TCA AAT CGC | 35 |
| | CTG CAG GTA A | |
| H44A(−07+15) | GAT CTG TCA AAT CGC | 36 |
| | CTG CAG G | |
| H44M(−07+17) | CAG ATC TGT CAA ATC | 37 |
| | GCC TGC AGG | |
| H44A(−08+15) | GAT CTG TCA AAT CGC | 38 |
| | CTG CAG GT | |
| H44A(−06+15) | GAT CTG TCA AAT CGC | 39 |
| | CTG CAG | |
| H44A(−08+17) | CAG ATC TGT CAA ATC | 40 |
| | GCC TGC AGG T | |
| H44A(−06+17) | CAG ATC TGT CAA ATC | 41 |
| | GCC TGC AG | |
| H50D(+04−18) | GGG ATC CAG TAT ACT | 42 |
| | TAC AGG C | |
| H50D(+07−18) | GGG ATC CAG TAT ACT | 43 |
| | TAC AGG CTC C | |
| H50D(+07−16) | GAT CCA GTA TAC TTA | 44 |
| | CAG GCT CC | |
| H50D(+07−17) | GGA TCC AGT ATA CTT | 45 |
| | ACA GGC TCC | |
| H50A(−19+07) | ACT TCC TCT TTA ACA | 46 |
| | GAA AAG CAT AC | |
| H50D(+07−15) | ATC CAG TAT ACT TAC | 47 |
| | AGG CTC C | |
| H50A(−02+23) | GAG CTC AGA TCT TCT | 48 |
| | AAC TTC CTC T | |
| H50D(+06−18) | GGG ATC CAG TAT ACT | 49 |
| | TAC AGG CTC | |
| H50D(+07−20) | ATG GGA TCC AGT ATA | 50 |
| | CTT ACA GGC TCC | |
| H52A(−01+24) | CTG TTC CAA ATC CTG | 51 |
| | CAT TGT TGC C | |

wherein each T of each of SEQ ID NOS: 1-51 is thymine or uracil. In certain aspects, each T in the base sequence is thymine.

In some embodiments, the antisense oligonucleotide or antisense oligonucleotide conjugate contains a T′ moiety attached to the 5′ end of the nucleic acid analog, wherein the T moiety is selected from:

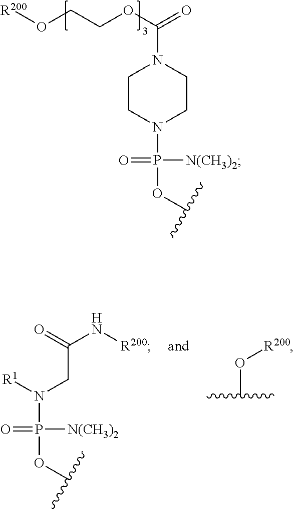

and herein R²⁰⁰ is hydrogen or a cell-penetrating peptide and R¹ is C₁-C₆ alkyl. In certain aspects, R²⁰⁰ is hydrogen.

In some embodiments, the nucleobases of the antisense oligonucleotide or antisense oligonucleotide are linked to morpholino ring structures. In certain aspects, the morpholino subunits are joined by phosphorous-containing intersubunit linkages joining a morpholino nitrogen of one subunit to a 5′ exocyclic carbon of an adjacent morpholino subunit.

In some embodiments, the antisense oligonucleotide or antisense oligonucleotide conjugate, is provided in a pharmaceutical composition formed by dissolving 0.005 mg/kg to about 300 mg/kg of the antisense oligonucleotide conjugate, or pharmaceutically acceptable salt thereof, in an aqueous carrier solution.

In some embodiments, the therapeutically effective amount of the antisense oligonucleotide or antisense oligonucleotide conjugate, or pharmaceutically acceptable salt thereof, is in an amount from about 0.005 mg/kg to about 300 mg/kg. In certain aspects, the therapeutically effective amount of the antisense oligonucleotide or antisense oligonucleotide conjugate, or pharmaceutically acceptable salt thereof, is at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg. In certain aspects, the therapeutically effective amount of the antisense oligonucleotide or antisense oligonucleotide conjugate, or pharmaceutically acceptable salt thereof, is about 0.005 mg/kg to about 200 mg/kg, about 0.1 mg/kg to about 100 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, or about 80 mg/kg to about 300 mg/kg. In certain aspects, the therapeutically effective amount of the antisense oligonucleotide or antisense oligonucleotide conjugate, or pharmaceutically acceptable salt thereof, is about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg.

In some embodiments, the antisense oligonucleotide or antisense oligonucleotide conjugate, or pharmaceutically acceptable salt thereof, is administered intravenously.

Methods of treating a human patient having Duchenne muscular dystrophy, the methods comprising administering to the human patient a pharmaceutical composition comprising a therapeutically effective amount of an antisense oligonucleotide or antisense oligonucleotide conjugate according to Formula (I):

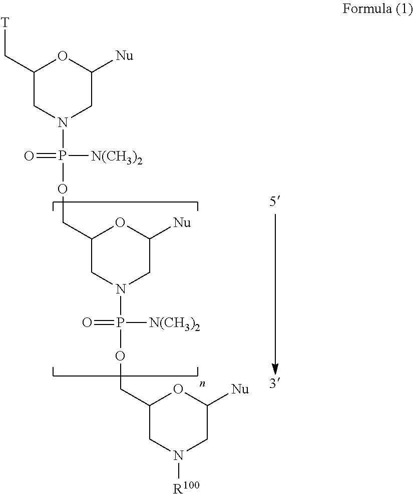

or a pharmaceutically acceptable salt thereof, wherein:
each Nu is a nucleobase which taken together form a targeting sequence;
T′ in Formula (I) is a moiety selected from:

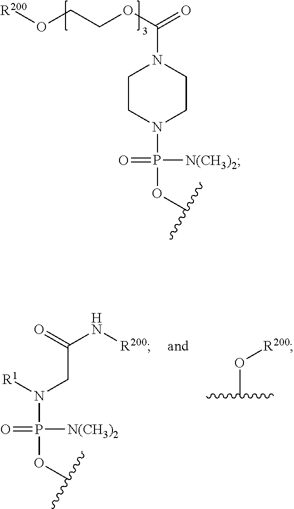

R¹⁰⁰ and R²⁰⁰ are each independently hydrogen or a cell-penetrating peptide and R¹ is C₁-C₆ alkyl;
each Nu from 1 to (n+1) and 5′ to 3′ corresponds to the nucleobases in one of the following:

| | SEQ |
|---|---|
| Base Sequence | ID |
| [5′ to 3′] | NO. |
| CTC CAA CAT CAA GGA | 1 |
| AGA TGG CAT TTC TAG | |
| ACC TCC AAC ATC AAG | 2 |
| GAA GAT GGC | |
| GTA CCT CCA ACA TCA | 3 |
| AGG AAG ATG GCA TTT | |
| GTA CCT CCA ACA TCA | 4 |
| AGG AAG ATG GCA T | |
| TCA AGG AAG ATG GCA | 5 |
| TTT CT | |
| UCA AGG AmAGm AmUGm | 6 |
| GmCA UUU CU | |
| GTT GCC TCC GGT TCT | 7 |
| GAA GGT GTT C | |
| GTT G5mC5mC T5mC5mC | 8 |
| GGT T5mC T GAA GGT G | |
| TT 5mC | |
| CCT CCG GTT CTG AAG | 9 |
| GTG TTC | |
| CTG AAG GTG TTC TTG | 10 |
| TAC TTC ATC C | |
| CCT CCG GTT CTG AAG | 11 |
| GTG TTC TTG T | |
| GTT GCC TCC GGT TCT | 12 |
| GAA GGT GTT CTT G | |
| TTG CCT CCG GTT CTG | 13 |
| AAG GTG TTC TTG TAC | |
| CTG TTG CCT CCG GTT | 14 |
| CTG AAG GTG | |
| CAT TCA ACT GTT GCC | 15 |
| TCC GGT TCT GAA GGT | |
| G | |
| CTG TTG CCT CCG GTT | 16 |
| CTG | |
| CAA TGC CAT CCT GGA | 17 |
| GTT CCT G | |
| GCT GCC CAA TGC CAT | 18 |
| CCT GGA GTT CCT GTA | |
| AGA T | |
| GCT GCC CAA TGC CAT | 19 |
| CCT GGA GTT CCT G | |
| GCT GCC CAA TGC CAT | 20 |
| CCT GGA GTT CCT GTA | |
| A | |
| CAA TGC CAT CCT GGA | 21 |
| GTT CCT GTA AGA TAC | |
| C | |
| CAA TGC CAT CCT GGA | 22 |
| GTT CCT GTA AGA T | |
| TGC CAT CCT GGA GTT | 23 |
| CCT GTA AGA TAC C | |
| GCT GCC CAA TGC CAT | 24 |
| CCT GGA GTT CCT GTA | |
| AGA | |
| TAC CAA | |
| CAA TGC CAT CCT GGA | 25 |
| GTT CCT GTA AGA | |
| GCT GCC CAA TGC CAT | 26 |
| CCT GGA GTT CCT GTA | |
| AG | |
| GCC CAA TGC CAT CCT | 27 |
| GGA GTT CCT GTA AGA | |
| TAC C | |
| GCC CAA TGC CAT CCT | 28 |
| GGA GTT CCT GTA AGA | |
| T | |
| TTG CCG CTG CCC AAT | 29 |
| GCC ATC CTG GAG TTC | |
| CTG TAA | |
| GAT | |
| GCC CAA TGC CAT CCT | 30 |
| GGA GTT CCT GTA A | |
| GCC GCT GCC CAA TGC | 31 |
| CAT CCT GGA GTT CCT | |
| GTA A | |
| GCC CAA TGC CAT CCT | 32 |
| GGA GTT CCT G | |
| GCC GCT GCC CAA TGC | 33 |
| CAT CCT GGA GTT CCT | |
| G | |
| GAT CTG TCA AAT CGC | 35 |
| CTG C AG GTA A | |
| GAT CTG TC A AAT CGC | 36 |
| CTG CAG G | |
| CAG ATC TGT CAA ATC | 37 |
| GCC TGC AGG | |
| GAT CTG TCA AAT CGC | 38 |
| CTG CAG GT | |
| GAT CTG TC A AAT CGC | 39 |
| CTG C AG | |
| CAG ATC TGT CAA ATC | 40 |
| GCC TGC AGG T | |
| CAG ATC TGT CAA ATC | 41 |
| GCC TGC AG | |
| GGG ATC CAG TAT ACT | 42 |
| TAC AGG C | |
| GGG ATC CAG TAT ACT | 43 |
| TAC AGG CTC C | |
| GAT CCA GTA TAC TTA | 44 |
| CAG GCT CC | |
| GGA TCC AGT ATA CTT | 45 |
| ACA GGC TCC | |
| ACT TCC TCT TTA ACA | 46 |
| GAA AAG CAT AC | |
| ATC CAG TAT ACT TAC | 47 |
| AGG CTC C | |
| GAG CTC AGA TCT TCT | 48 |
| AAC TTC CTC T | |
| GGG ATC CAG TAT ACT | 49 |
| TAC AGG CTC | |
| ATG GGA TCC AGT ATA | 50 |
| CTT ACA GGC TCC | |
| CTG TTC CAA ATC CTG | 51 |
| CAT TGT TGC C | |

wherein each T of each of SEQ ID NOS: 1-51 is thymine or uracil are also provided herein. In certain aspects, each Nu from 1 to (n+1) and 5′ to 3′ of the antisense oligonucleotide or antisense oligonucleotide conjugate corresponds to SEQ ID NO: 1, SEQ ID NO: 7, or SEQ ID NO: 17.

In some embodiments, T′ in the antisense oligonucleotide or antisense oligonucleotide conjugate of Formula (I) is a moiety selected from:

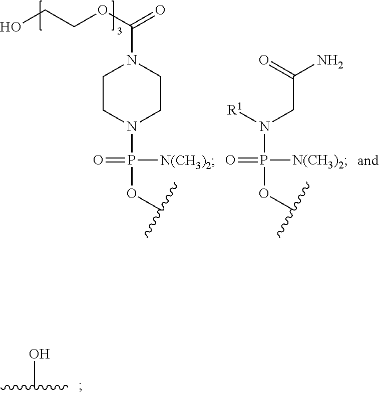

R¹⁰⁰ is a cell penetrating peptide and R¹ is C₁-C₆ alkyl. In certain aspects, T′ in the antisense oligonucleotide conjugate of Formula (I) is

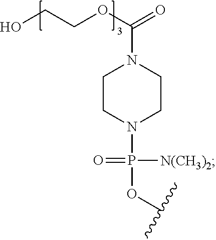

and R¹⁰⁰ is a cell penetrating peptide.

In some embodiments, the cell penetrating peptide of the antisense oligonucleotide conjugate is an arginine-rich peptide. In certain aspects, the arginine-rich peptide of the antisense oligonucleotide conjugate is selected from the group consisting of —(RXR)₄—R^{a} (SEQ ID NO: 52), R—(FFR)₃—R^{a} (SEQ ID NO: 53), —B—X—(RXR)₄—R^{a} (SEQ ID NO: 54), —B—X—R—(FFR)₃—R^{a} (SEQ ID NO: 55), -GLY-R—(FFR)₃—R^{a} (SEQ ID NO: 56), -GLY-R₅—R^{a} (SEQ ID NO: 59), —R₅—R^{a} (SEQ ID NO: 60), -GLY-R₆—R^{a} (SEQ ID NO: 57) and —R₆—R^{a} (SEQ ID NO: 58), wherein R^{a} is selected from H, acyl, benzoyl, and stearoyl, and wherein R is arginine, X is 6-aminohexanoic acid, B is β-alanine, F is phenylalanine and GLY (or G) is glycine. In certain aspects, the arginine-rich peptide of the antisense oligonucleotide conjugate is —R₅—R^{a} (SEQ ID NO: 60) or —R₆—R^{a} (SEQ ID NO: 58), wherein R^{a} is an acyl group. In certain aspects, the arginine-rich peptide of the antisense oligonucleotide conjugate is —R₆—R^{a} (SEQ ID NO: 58), wherein R^{a} is an acyl group. In certain aspects, the arginine-rich peptide of the antisense oligonucleotide conjugate is -GLY-R₅—R^{a} (SEQ ID NO: 59) or -GLY-R₆—R^{a} (SEQ ID NO: 57), wherein R^{a} is an acyl group. In certain aspects, the arginine-rich peptide is -GLY-R₆—R^{a} (SEQ ID NO: 57), wherein R^{a} is an acyl group. In certain aspects, the antisense oligonucleotide conjugate comprises a cell penetrating peptide that is an arginine-rich peptide that is —R₅—R^{a} (SEQ ID NO: 60) or —R₆—R^{a} (SEQ ID NO: 58), wherein R^{a} is H. In certain aspects, the antisense oligonucleotide conjugate comprises a cell penetrating peptide that is an arginine-rich peptide that is —R₆—R^{a} (SEQ ID NO: 58), wherein R^{a} H. In certain aspects, the arginine-rich peptide is -GLY-R₅—R^{a} (SEQ ID NO: 59) or -GLY-R₆—R^{a} (SEQ ID NO: 57), wherein R^{a} is H. In certain aspects, the arginine-rich peptide is -GLY-R₆—R^{a} (SEQ ID NO: 57), wherein R^{a} is H.

In some embodiments, the antisense oligonucleotide or antisense oligonucleotide conjugate is in free base form. In some embodiments, the antisense oligonucleotide or antisense oligonucleotide conjugate is a pharmaceutically acceptable salt. In some embodiments, the antisensense oligonucleotide conjugate is a halide salt (e.g., HCl salt). In some aspects, the antisense oligonucleotide conjugate is a monohalide, dihalide, trihalide, tetrahalide, pentahalide, or hexahalide salt. In certain aspects, the antisense oligonucleotide conjugate is an HCl salt. In certain aspects, the HCl salt of the antisense oligonucleotide or antisense oligonucleotide conjugate is a 1HCl, 2HCl, 3HCl, 4HCl, 5HCl, or 6HCl salt. In some embodiments, the antisense oligonucleotide or antisense oligonucleotide conjugate is provided as a mixture of free base and salt form.

In some embodiments, the antisense oligonucleotide is eteplirsen, golodirsen, or casimersen. In some embodiments, the antisense oligonucleotide conjugate is PPPMO #1, PPMO #2, or PPMO #3, or a pharmaceutically acceptable salt thereof. In some embodiments, the antisense oligonucleotide conjugate is PPMO #1.6HCl, PPMO #2.6HCl, or PPMO #3.6HCl.

In some embodiments, the antisense oligonucleotide conjugate, or a pharmaceutically acceptable salt thereof, is provided in a pharmaceutical composition formed by dissolving 0.005 mg/kg to about 300 mg/kg of the antisense oligonucleotide conjugate, or pharmaceutically acceptable salt thereof, in an aqueous carrier solution In some embodiments, the pharmaceutical composition is formed by dissolving about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #1 in an aqueous carrier solution. In some embodiments, the pharmaceutical composition is formed by dissolving 20 mg/kg, 30 mg/kg, 40 mg/kg, 50 mg/kg, 60 mg/kg, 70 mg/kg, 80 mg/kg, 90 mg/kg, or 100 mg/kg of PPMO #1.6HCl in an aqueous carrier solution. In some embodiments, the pharmaceutical composition is formed by dissolving about 60 mg/kg of a pharmaceutically acceptable salt of PPMO #1 in an aqueous carrier solution. In some embodiments, the pharmaceutical composition is formed by dissolving about 80 mg/kg of a pharmaceutically acceptable salt of PPMO #1 in an aqueous carrier solution. In some embodiments, the pharmaceutical composition is formed by dissolving about 100 mg/kg of a pharmaceutically acceptable salt of PPMO #1 in an aqueous carrier solution. In some embodiments, the pharmaceutical composition is formed by dissolving about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #2 in an aqueous carrier solution. In some embodiments, the pharmaceutical composition is formed by dissolving about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #3 in an aqueous carrier solution.

In some embodiments, the therapeutically effective amount of the antisense oligonucleotide or antisense oligonucleotide conjugate, or pharmaceutically acceptable salt thereof, is in an amount from about 0.005 mg/kg to about 300 mg/kg. In certain aspects, the therapeutically effective amount of the antisense oligonucleotide or antisense oligonucleotide conjugate, or pharmaceutically acceptable salt thereof, is at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg. In certain aspects, the therapeutically effective amount of the antisense oligonucleotide or antisense oligonucleotide conjugate, or pharmaceutically acceptable salt thereof, is about 0.005 mg/kg to about 200 mg/kg, about 0.1 mg/kg to about 100 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, or about 80 mg/kg to about 300 mg/kg. In certain aspects, the therapeutically effective amount of the antisense oligonucleotide or antisense oligonucleotide conjugate, or pharmaceutically acceptable salt thereof, is about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg. In some embodiments, a therapeutically effective amount of a pharmaceutically acceptable salt of PPMO #1 is about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg. In some embodiments, a therapeutically effective amount of a pharmaceutically acceptable salt of PPMO #1 is about 30 mg/kg, about 60 mg/kg, about 80 mg/kg, or about 100 mg/kg. In some embodiments, a therapeutically effective amount of the pharmaceutical composition is 20 mg/kg, 30 mg/kg, 40 mg/kg, 50 mg/kg, 60 mg/kg, 70 mg/kg, 80 mg/kg, 90 mg/kg, or 100 mg/kg of PPMO #1.6HCl. In some embodiments, a therapeutically effective amount of a pharmaceutically acceptable salt of PPMO #2 is about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg. In some embodiments, a therapeutically effective amount of a pharmaceutically acceptable salt of PPMO #3 is about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg.

In some embodiments, the antisense oligonucleotide or antisense oligonucleotide conjugate, or pharmaceutically acceptable salt thereof, is administered intravenously.

Methods of treating a human patient having Duchenne muscular dystrophy, the methods comprising administering to the human patient a pharmaceutical composition comprising a therapeutically effective amount of an antisense oligonucleotide conjugate according to Formula (II):

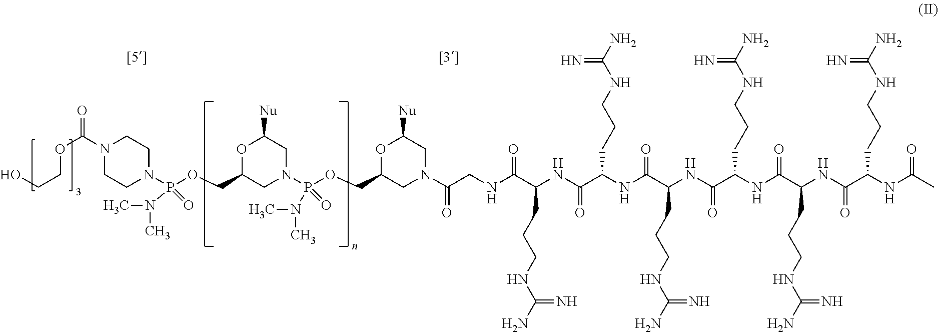

or a pharmaceutically acceptable salt thereof, each Nu from 1 to (n+1) and 5′ to 3′ corresponds to the nucleobases in one of the following:

| | SEQ |
|---|---|
| Base Sequence | ID |
| [5′ to 3′] | NO. |
| CTC CAA CAT CAA GGA | 1 |
| AGA TGG CAT TTC TAG | |
| ACC TCC AAC ATC AAG | 2 |
| GAA GAT GGC | |
| GTA CCT CCA ACA TCA | 3 |
| AGG AAG ATG GCA TTT | |
| GTA CCT CCA ACA TCA | 4 |
| AGG AAG ATG GCA T | |
| TCA AGG AAG ATG GCA | 5 |
| TTT CT | |
| UCA AGG AmAGm AmUGm | 6 |
| GmCA UUU CU | |
| GTT GCC TCC GGT TCT | 7 |
| GAA GGT GTT C | |
| GTT G5mC5mC T5mC5mC | 8 |
| GGT T5mC T GAA GGT | |
| GTT 5mC | |
| CCT CCG GTT CTG AAG | 9 |
| GTG TTC | |
| CTG AAG GTG TTC TTG | 10 |
| TAC TTC ATC C | |
| CCT CCG GTT CTG AAG | 11 |
| GTG TTC TTG T | |
| GTT GCC TCC GGT TCT | 12 |
| GAA GGT GTT CTT G | |
| TTG CCT CCG GTT CTG | 13 |
| AAG GTG TTC TTG TAC | |
| CTG TTG CCT CCG GTT | 14 |
| CTG AAG GTG | |
| CAT TCA ACT GTT GCC | 15 |
| TCC GGT TCT GAA GGT | |
| G | |
| CTG TTG CCT CCG GTT | 16 |
| CTG | |
| CAA TGC CAT CCT GGA | 17 |
| GTT CCT G | |
| GCT GCC CAA TGC CAT | 18 |
| CCT GGA GTT CCT GTA | |
| AGA T | |
| GCT GCC CAA TGC CAT | 19 |
| CCT GGA GTT CCT G | |
| GCT GCC CAA TGC CAT | 20 |
| CCT GGA GTT CCT GTA | |
| A | |
| CAA TGC CAT CCT GGA | 21 |
| GTT CCT GTA AGA TAC | |
| C | |
| CAA TGC CAT CCT GGA | 22 |
| GTT CCT GTA AGA T | |
| TGC CAT CCT GGA GTT | 23 |
| CCT GTA AGA TAC C | |
| GCT GCC CAA TGC CAT | 24 |
| CCT GGA GTT CCT GTA | |
| AGA | |
| TAC CAA | |
| CAA TGC CAT CCT GGA | 25 |
| GTT CCT GTA AGA | |
| GCT GCC CAA TGC CAT | 26 |
| CCT GGA GTT CCT GTA | |
| AG | |
| GCC CAA TGC CAT CCT | 27 |
| GGA GTT CCT GTA AGA | |
| T AC C | |
| GCC CAA TGC CAT CCT | 28 |
| GGA GTT CCT GTA AGA | |
| T | |
| TTG CCG CTG CCC AAT | 29 |
| GCC ATC CTG GAG TTC | |
| CTG TAA GAT | |
| GCC CAA TGC CAT CCT | 30 |
| GGA GTT CCT GTA A | |
| GCC GCT GCC CAA TGC | 31 |
| CAT CCT GGA GTT CCT | |
| GTA A | |
| GCC CAA TGC CAT CCT | 32 |
| GGA GTT CCT G | |
| GCC GCT GCC C AA TGC | 33 |
| C AT CCT GGA GTT CC | |
| T G | |
| m5C-G-m5C-T-G-C-m5C- | 34 |
| m5C-A-A-T-G-m5C-m5C- | |
| A-U-m5C-m5C | |
| GAT CTG TCA AAT CGC | 35 |
| CTG CAG GTA A | |
| GAT CTG TCA AAT CGC | 36 |
| CTG CAG G | |
| CAG ATC TGT CAA ATC | 37 |
| GCC TGC AGG | |
| GAT CTG TCA AAT CGC | 38 |
| CTG CAG GT | |
| GAT CTG TCA AAT CGC | 39 |
| CTG CAG | |
| CAG ATC TGT CAA ATC | 40 |
| GCC TGC AGG T | |
| CAG ATC TGT CAA ATC | 41 |
| GCC TGC AG | |
| GGG ATC CAG TAT ACT | 42 |
| TAC AGG C | |
| GGG ATC CAG TAT ACT | 43 |
| TAC AGG CTC C | |
| GAT CCA GTA TAC TTA | 44 |
| CAG GCT CC | |
| GGA TCC AGT ATA CTT | 45 |
| ACA GGC TCC | |
| ACT TCC TCT TTA ACA | 46 |
| GAA AAG CAT AC | |
| ATC CAG TAT ACT TAC | 47 |
| AGG CTC C | |
| GAG CTC AGA TCT TCT | 48 |
| AAC TTC CTC T | |
| GGG ATC CAG TAT ACT | 49 |
| TAC AGG CTC | |
| ATG GGA TCC AGT ATA | 50 |
| CTT ACA GGC TCC | |
| CTG TTC CAA ATC CTG | 51 |
| CAT TGT TGC C | |

wherein A is

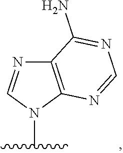 [SMILES: *n1cnc2c(N)ncnc21]

### C is

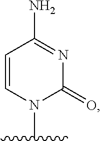 [SMILES: *n1ccc(N)nc1=O]

### G is

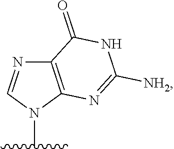 [SMILES: *n1cnc2c(=O)[nH]c(N)nc21]

### T is

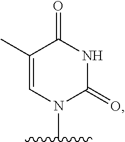 [SMILES: *n1cc(C)c(=O)[nH]c1=O]

### U is

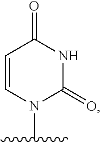 [SMILES: *n1ccc(=O)[nH]c1=O]

Gm is methylated guanine, Am is methylated adenine, and m5C is

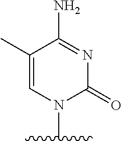 [SMILES: *n1cc(C)c(N)nc1=O]

are provided herein. In certain aspects, each Nu from 1 to (n+1) and 5′ to 3′ of the antisense oligonucleotide conjugate of Formula (II) corresponds to SEQ ID NO: 1, SEQ ID NO: 7, or SEQ ID NO: 17.

In some embodiments, the antisense oligonucleotide conjugate is in free base form. In some embodiments, the antisense oligonucleotide conjugate is a pharmaceutically acceptable salt. In certain aspects, the antisense oligonucleotide is a halide salt. In certain aspects, the antisense oligonucleotide conjugate is a hexahalide salt. In certain aspects, the antisense oligonucleotide conjugate is an HCl salt. In certain aspects, the antisense oligonucleotide conjugate is a 6HCl salt. In some embodiments, the antisense oligonucleotide conjugate is provided as a mixture of free base and salt form.

In some embodiments, the antisense oligonucleotide conjugate is provided in a pharmaceutical composition formed by dissolving 0.005 mg/kg to about 300 mg/kg of the antisense oligonucleotide conjugate, or pharmaceutically acceptable salt thereof, in an aqueous carrier solution.

In some embodiments, the therapeutically effective amount of the antisense oligonucleotide conjugate, or pharmaceutically acceptable salt thereof, is in an amount from about 0.005 mg/kg to about 300 mg/kg. In certain aspects, the therapeutically effective amount of the antisense oligonucleotide or antisense oligonucleotide conjugate, or pharmaceutically acceptable salt thereof, is at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg. In certain aspects, the therapeutically effective amount of the antisense oligonucleotide or antisense oligonucleotide conjugate, or pharmaceutically acceptable salt thereof, is about 0.005 mg/kg to about 200 mg/kg, about 0.1 mg/kg to about 100 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, or about 80 mg/kg to about 300 mg/kg. In certain aspects, the therapeutically effective amount of the antisense oligonucleotide or antisense oligonucleotide conjugate, or pharmaceutically acceptable salt thereof, is about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg. In some embodiments, a therapeutically effective amount of a pharmaceutically acceptable salt of PPMO #1 is about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg. In some embodiments, a therapeutically effective amount of a pharmaceutically acceptable salt of PPMO #1 is about 30 mg/kg, about 60 mg/kg, about 80 mg/kg, or about 100 mg/kg. In some embodiments, a therapeutically effective amount of the pharmaceutical composition is 20 mg/kg, 30 mg/kg, 40 mg/kg, 50 mg/kg, 60 mg/kg, 70 mg/kg, 80 mg/kg, 90 mg/kg, or 100 mg/kg of PPMO #1.6HCl. In some embodiments, a therapeutically effective amount of a pharmaceutically acceptable salt of PPMO #2 is about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg. In some embodiments, a therapeutically effective amount of a pharmaceutically acceptable salt of PPMO #3 is about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg.

In some embodiments, the antisense oligonucleotide conjugate, or pharmaceutically acceptable salt thereof, is administered intravenously.

Methods of treating a human patient having Duchenne muscular dystrophy, the methods comprising administering to the human patient a pharmaceutical composition comprising a therapeutically effective amount of an antisense oligonucleotide conjugate according to Formula (IV):

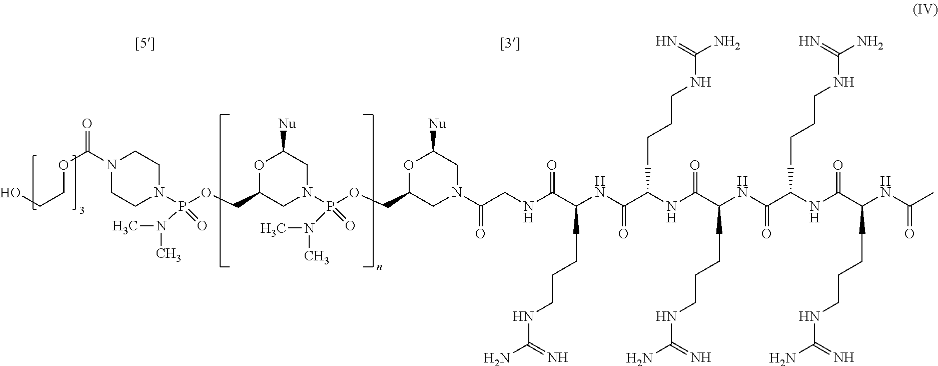

or a pharmaceutically acceptable salt thereof, where each Nu from 1 to (n+1) and 5′ to 3′ corresponds to the nucleobases in one of the following:

| | SEQ |
|---|---|
| Base Sequence | ID |
| [5′ to 3′] | NO. |
| CTC CAA CAT CAA GGA | 1 |
| AGA TGG CAT TTC TAG | |
| ACC TCC AAC ATC AAG | 2 |
| GAA GAT GGC | |
| GTA CCT CCA ACA TCA | 3 |
| AGG AAG ATG GCA TTT | |
| GTA CCT CCA ACA TCA | 4 |
| AGG AAG ATG GCA T | |
| TCA AGG AAG ATG GCA | 5 |
| TTT CT | |
| UCA AGG AmAGm AmUGm | 6 |
| GmCA UUU CU | |
| GTT GCC TCC GGT TCT | 7 |
| GAA GGT GTT C | |
| GTT G5mC5mC T5mC5mC | 8 |
| GGT T5mC T GAA GGT | |
| GTT 5mC | |
| CCT CCG GTT CTG AAG | 9 |
| GTG TTC | |
| CTG AAG GTG TTC TTG | 10 |
| TAC TTC ATC C | |
| CCT CCG GTT CTG AAG | 11 |
| GTG TTC TTG T | |
| GTT GCC TCC GGT TCT | 12 |
| GAA GGT GTT CTT G | |
| TTG CCT CCG GTT CTG | 13 |
| AAG GTG TTC TTG TAC | |
| CTG TTG CCT CCG GTT | 14 |
| CTG AAG GTG | |
| CAT TCA ACT GTT GCC | 15 |
| TCC GGT TCT GAA GGT | |
| G | |
| CTG TTG CCT CCG GTT | 16 |
| CTG | |
| CAA TGC CAT CCT GGA | 17 |
| GTT CCT G | |
| GCT GCC CAA TGC C AT | 18 |
| CCT GGA GIT CCT GTA | |
| AGA T | |
| GCT GCC CAA TGC CAT | 19 |
| CCT GGA GTT CCT G | |
| GCT GCC CAA TGC CAT | 20 |
| CCT GGA GTT CCT GTA | |
| A | |
| CAA TGC CAT CCT GGA | 21 |
| GTT CCT GTA AGA TAC | |
| C | |
| CAA TGC CAT CCT GGA | 22 |
| GTT CCT GT A AGA T | |
| TGC CAT CCT GGA GTT | 23 |
| CCT GTA AGA TAC C | |
| GCT GCC CAA TGC CAT | 24 |
| CCT GGA GTT CCT GTA | |
| AGA TAC | |
| CAA | |
| CAA TGC CAT CCT GGA | 25 |
| GTT CCT GTA AGA | |
| GCT GCC CAA TGC CAT | 26 |
| CCT GGA GTT CCT GTA | |
| AG | |
| GCC CAA TGC CAT CCT | 27 |
| GGA GTT CCT GTA AGA | |
| TAC C | |
| GCC CAA TGC CAT CCT | 28 |
| GGA GTT CCT GTA AGA | |
| T | |
| TTG CCG CTG CCC AAT | 29 |
| GCC ATC CTG GAG TTC | |
| CTG TAA | |
| GAT | |
| GCC CAA TGC CAT CCT | 30 |
| GGA GTT CCT GTA A | |
| GCC GCT GCC CAA TGC | 31 |
| CAT CCT GGA GTT CCT | |
| GTA A | |
| GCC CAA TGC CAT CCT | 32 |
| GGA GTT CCT G | |
| GCC GCT GCC CAA TGC | 33 |
| CAT CCT GGA GTT CCT | |
| G | |
| m5C-G-m5C-T-G-C-m5C- | 34 |
| m5C-A-A-T-G-m5C-m5C- | |
| A-U-m5C-m5C | |
| GAT CTG TCA AAT CGC | 35 |
| CTG CAG GTA A | |
| GAT CTG TCA AAT CGC | 36 |
| CTG CAG G | |
| CAG ATC TGT CAA ATC | 37 |
| GCC TGC AGG | |
| GAT CTG TCA AAT CGC | 38 |
| CTG CAG GT | |
| GAT CTG TCA AAT CGC | 39 |
| CTG CAG | |
| CAG ATC TGT CAA ATC | 40 |
| GCC TGC AGG T | |
| CAG ATC TGT CAA ATC | 41 |
| GCC TGC AG | |
| GGG ATC CAG TAT ACT | 42 |
| TAC AGG C | |
| GGG ATC CAG TAT ACT | 43 |
| TAC AGG CTC C | |
| GAT CCA GTA TAC TTA | 44 |
| CAG GCT CC | |
| GGA TCC AGT ATA CTT | 45 |
| ACA GGC TCC | |
| ACT TCC TCT TTA ACA | 46 |
| GAA AAG CAT AC | |
| ATC CAG TAT ACT TAC | 47 |
| AGG CTC C | |
| GAG CTC AGA TCT TCT | 48 |
| AAC TTC CTC T | |
| GGG ATC CAG TAT ACT | 49 |
| TAC AGG CTC | |
| ATG GGA TCC AGT ATA | 50 |
| CTT ACA GGC TCC | |
| CTG TTC CAA ATC CTG | 51 |
| CAT TGT TGC C | |

wherein A is

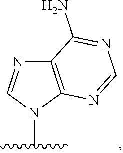 [SMILES: *n1cnc2c(N)ncnc21]

### C is

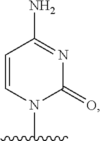 [SMILES: *n1ccc(N)nc1=O]

### G is

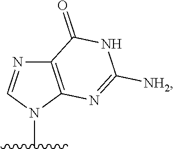 [SMILES: *n1cnc2c(=O)[nH]c(N)nc21]

### T is

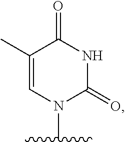 [SMILES: *n1cc(C)c(=O)[nH]c1=O]

### U is

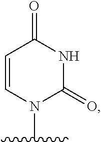 [SMILES: *n1ccc(=O)[nH]c1=O]

Gm is methylated guanine, Am is methylated adenine, and m5C is

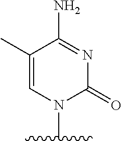 [SMILES: *n1cc(C)c(N)nc1=O]

are provided herein. In some aspects, each Nu from 1 to (n+1) and 5′ to 3′ of the antisense oligonucleotide conjugate of Formula (IV) corresponds to SEQ ID NO: 1, SEQ ID NO: 7, or SEQ ID NO: 17.

In some embodiments, the antisense oligonucleotide conjugate is in free base form. In some embodiments, the antisense oligonucleotide is a pharmaceutically acceptable salt thereof. In some embodiments, the antisense oligonucleotide conjugate is in the form of a halide salt. In some embodiments, the antisense oligonucleotide conjugate is in the form of a hexahalide salt form. In certain aspects, the antisense oligonucleotide conjugate is an HCl salt. In certain aspects, the HCl salt is a 5HCl salt. In certain aspects, the HCl salt is a 6HCl salt. In some embodiments, the antisense oligonucleotide conjugate is provided as a mixture of free base and salt form.

In some embodiments, the antisense oligonucleotide conjugate is provided in a pharmaceutical composition formed by dissolving 0.005 mg/kg to about 300 mg/kg of the antisense oligonucleotide conjugate, or pharmaceutically acceptable salt thereof, in an aqueous carrier solution.

In some embodiments, the therapeutically effective amount of the antisense oligonucleotide conjugate, or pharmaceutically acceptable salt thereof, is in an amount from about 0.005 mg/kg to about 300 mg/kg. In certain aspects, the therapeutically effective amount of the antisense oligonucleotide or antisense oligonucleotide conjugate, or pharmaceutically acceptable salt thereof, is at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg. In certain aspects, the therapeutically effective amount of the antisense oligonucleotide or antisense oligonucleotide conjugate, or pharmaceutically acceptable salt thereof, is about 0.005 mg/kg to about 200 mg/kg, about 0.1 mg/kg to about 100 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, or about 80 mg/kg to about 300 mg/kg. In certain aspects, the therapeutically effective amount of the antisense oligonucleotide or antisense oligonucleotide conjugate, or pharmaceutically acceptable salt thereof, is about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg.

In some embodiments, the antisense oligonucleotide conjugate, or pharmaceutically acceptable salt thereof, is administered intravenously.

Methods of treating a human patient having Duchenne muscular dystrophy, the method comprising administering to the human patient once every four weeks a therapeutically effective amount of an antisense oligonucleotide conjugate, or a pharmaceutically acceptable salt thereof, said the antisense oligonucleotide conjugate comprising a cell penetrating peptide covalently attached to an oligonucleotide; wherein said antisense oligonucleotide conjugate, or pharmaceutically acceptable salt thereof, induces exon skipping in the human dystrophin gene are provided herein.

In some embodiments, the antisense oligonucleotide conjugate, or pharmaceutically acceptable salt thereof, comprises a cell penetrating peptide that is an arginine-rich peptide. In some embodiments, the antisense oligonucleotide conjugate, or pharmaceutically acceptable salt thereof, comprises a cell penetrating peptide that is an arginine-rich peptide that is -GLY-R₅—R^{a} (SEQ ID NO: 59), —R₅—R^{a} (SEQ ID NO: 60), -GLY-R₆—R^{a} (SEQ ID NO: 57) or —R₆—R^{a} (SEQ ID NO: 58), wherein R is arginine and R^{a} is hydrogen or an acyl group.

In some embodiments, the antisense oligonucleotide conjugate, or pharmaceutically acceptable salt thereof, induces skipping of exon 44, exon 45, exon 50, exon 51, exon 52, or exon 53 target region of the dystrophin pre-mRNA. In some embodiments, the antisense oligonucleotide conjugate is according to Formula (I):

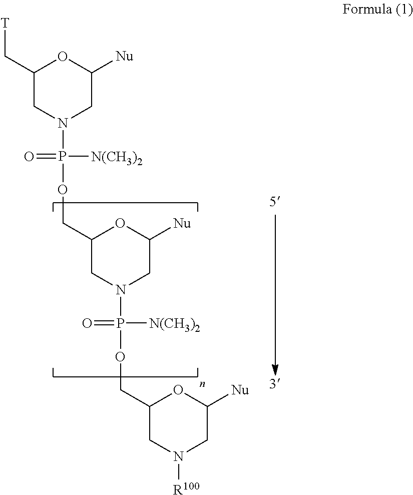

or a pharmaceutically acceptable salt thereof, wherein:
each Nu is a nucleobase which taken together form a targeting sequence;
T′ in Formula (I) is a moiety selected from:

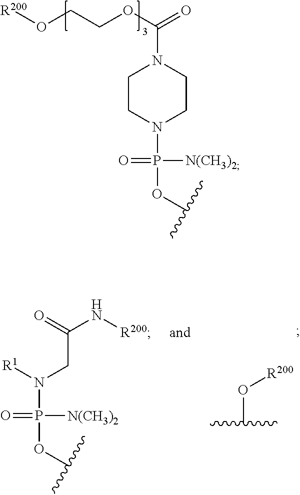

R¹⁰⁰ is a cell-penetrating peptide;
R²⁰⁰ is hydrogen;
R¹ is C₁-C₆ alkyl; and
each Nu from 1 to (n+1) and 5′ to 3′ corresponds to the nucleobases in one of the following:

| | SEQ |
|---|---|
| Base Sequence | ID |
| [5′ to 3′] | NO. |
| CTC CAA CAT CAA GGA | 1 |
| AGA TGG CAT TTC TAG | |
| ACC TCC AAC ATC AAG | 2 |
| GAA GAT GGC | |
| GTA CCT CCA ACA TCA | 3 |
| AGG AAG ATG GCA TTT | |
| GTA CCT CCA ACA TCA | 4 |
| AGG AAG ATG GCA T | |
| TCA AGG AAG ATG GCA | 5 |
| TTT CT | |
| UCA AGG AmAGm AmUGm | 6 |
| GmCA UUU CU | |
| GTT GCC TCC GGT TCT | 7 |
| GAA GGT GTT C | |
| GTT G5mC5mC T5mC5mC | 8 |
| GGT T5mC T GAA GGT | |
| GTT 5mC | |
| CCT CCG GTT CTG AAG | 9 |
| GTG TTC | |
| CTG AAG GTG TTC TTG | 10 |
| TAC TTC ATC C | |
| CCT CCG GTT CTG AAG | 11 |
| GTG TTC TTG T | |
| GTT GCC TCC GGT TCT | 12 |
| GAA GGT GTT CTT G | |
| TTG CCT CCG GTT CTG | 13 |
| AAG GTG TTC TTG TAC | |
| CTG TTG CCT CCG GTT | 14 |
| CTG AAG GTG | |
| CAT TCA ACT GTT GCC | 15 |
| TCC GGT TCT GAA GGT | |
| G | |
| CTG TTG CCT CCG GTT | 16 |
| CTG | |
| CAA TGC CAT CCT GGA | 17 |
| GTT CCT G | |
| GCT GCC CAA TGC CAT | 18 |
| CCT GGA GTT CCT GTA | |
| AGA T | |
| GCT GCC CAA TGC CAT | 19 |
| CCT GGA GTT CCT G | |
| GCT GCC CAA TGC CAT | 20 |
| CCT GGA GTT CCT GTA | |
| A | |
| CAA TGC CAT CCT GGA | 21 |
| GTT CCT GTA AGA TAC | |
| C | |
| CAA TGC CAT CCT GGA | 22 |
| GTT CCT GTA AGA T | |
| TGC CAT CCT GGA GTT | 23 |
| CCT GTA AGA TAC C | |
| GCT GCC CAA TGC CAT | 24 |
| CCT GGA GTT CCT GTA | |
| AGA TAC CAA | |
| CAA TGC CAT CCT GGA | 25 |
| GTT CCT GTA AGA | |
| GCT GCC CAA TGC CAT | 26 |
| CCT GGA GTT CCT GTA | |
| AG | |
| GCC CAA TGC CAT CCT | 27 |
| GGA GTT CCT GTA AGA | |
| TAC C | |
| GCC CAA TGC CAT CCT | 28 |
| GGA GTT CCT GTA AGA | |
| T | |
| TTG CCG CTG CCC AAT | 29 |
| GCC ATC CTG GAG TTC | |
| CTG TAA GAT | |
| GCC CAA TGC CAT CCT | 30 |
| GGA GTT CCT GTA A | |
| GCC GCT GCC CAA TGC | 31 |
| CAT CCT GGA GTT CCT | |
| GTA A | |
| GCC CAA TGC CAT CCT | 32 |
| GGA GTT CCT G | |
| GCC GCT GCC CAA TGC | 33 |
| CAT CCT GGA GTT CCT | |
| G | |
| m5C-G-m5C-T-G-C-m5C- | 34 |
| m5C-A-A-T-G-m5C-m5C- | |
| A-U-m5C-m5C | |
| GAT CTG TCA AAT CGC | 35 |
| CTG CAG GTA A | |
| GAT CTG TCA AAT CGC | 36 |
| CTG CAG G | |
| CAG ATC TGT CAA ATC | 37 |
| GCC TGC AGG | |
| GAT CTG TCA AAT CGC | 38 |
| CTG CAG GT | |
| GAT CTG TCA AAT CGC | 39 |
| CTG CAG | |
| CAG ATC TGT CAA ATC | 40 |
| GCC TGC AGG T | |
| CAG ATC TGT CAA ATC | 41 |
| GCC TGC AG | |
| GGG ATC CAG TAT ACT | 42 |
| TAC AGG C | |
| GGG ATC CAG TAT ACT | 43 |
| TAC AGG CTC C | |
| GAT CCA GTA TAC TTA | 44 |
| CAG GCT CC | |
| GGA TCC AGT ATA CTT | 45 |
| ACA GGC TCC | |
| ACT TCC TCT TTA ACA | 46 |
| GAA AAG CAT AC | |
| ATC CAG TAT ACT TAC | 47 |
| AGG CTC C | |
| GAG CTC AGA TCT TCT | 48 |
| AAC TTC CTC T | |
| GGG ATC CAG TAT ACT | 49 |
| TAC AGG CTC | |
| ATG GGA TCC AGT ATA | 50 |
| CTT ACA GGC TCC | |
| CTG TTC CAA ATC CTG | 51 |
| CAT TGT TGC C | |

wherein each T of each of SEQ ID NOS: 1-51 is thymine or uracil.

In some embodiments, each Nu from 1 to (n+1) and 5′ to 3′ of the antisense oligonucleotide conjugate, or pharmaceutically acceptable salt thereof, corresponds to SEQ ID NO: 1, SEQ ID NO: 7, or SEQ ID NO: 17.

In some embodiments, the antisense oligonucleotide conjugate is PPPMO #1, PPMO #2, or PPMO #3, or a pharmaceutically acceptable salt thereof.

In some embodiments, the method comprises administering to the human patient a therapeutically effective amount of an antisense oligonucleotide conjugate according to Formula (II):

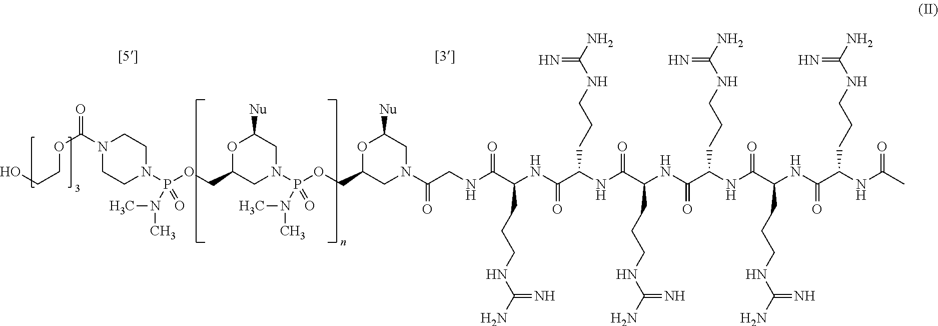

or a pharmaceutically acceptable salt thereof, each Nu from 1 to (n+1) and 5′ to 3′ corresponds to the nucleobases in one of the following:

| | SEQ |
|---|---|
| Base Sequence | ID |
| [5′ to 3′] | NO. |
| CTC CAA CAT CAA GGA | 1 |
| AGA TGG CAT TTC TAG | |
| ACC TCC AAC ATC AAG | 2 |
| GAA GAT GGC | |
| GTA CCT CCA ACA TCA | 3 |
| AGG AAG ATG GCA TTT | |
| GTA CCT CCA ACA TCA | 4 |
| AGG AAG ATG GCA T | |
| TCA AGG AAG ATG GCA | 5 |
| TTT CT | |
| UCA AGG AmAGm AmUGm | 6 |
| GmCA UUU CU | |
| GTT GCC TCC GGT TCT | 7 |
| GAA GGT GTT C | |
| GTT G5mC5mC T5mC5mC | 8 |
| GGT T5mC T GAA GGT G | |
| TT 5mC | |
| CCT CCG GTT CTG AAG | 9 |
| GTG TTC | |
| CTG AAG GTG TTC TTG | 10 |
| TAC TTC ATC C | |
| CCT CCG GTT CTG AAG | 11 |
| GTG TTC TTG T | |
| GTT GCC TCC GGT TCT | 12 |
| GAA GGT GTT CTT G | |
| TTG CCT CCG GTT CTG | 13 |
| AAG GTG TTC TTG TAC | |
| CTG TTG CCT CCG GTT | 14 |
| CTG AAG GTG | |
| CAT TCA ACT GTT GCC | 15 |
| TCC GGT TCT GAA GGT | |
| G | |
| CTG TTG CCT CCG GTT | 16 |
| CTG | |
| CAA TGC CAT CCT GGA | 17 |
| GTT CCT G | |
| GCT GCC CAA TGC CAT | 18 |
| CCT GGA GTT CCT GTA | |
| AGA T | |
| GCT GCC CAA TGC CAT | 19 |
| CCT GGA GTT CCT G | |
| GCT GCC CAA TGC CAT | 20 |
| CCT GGA GTT CCT GTA | |
| A | |
| CAA TGC CAT CCT GGA | 21 |
| GTT CCT GTA AGA TAC | |
| C | |
| CAA TGC CAT CCT GGA | 22 |
| GTT CCT GTA AGA T | |
| TGC CAT CCT GGA GTT | 23 |
| CCT GTA AGA TAC C | |
| GCT GCC CAA TGC CAT | 24 |
| CCT GGA GTT CCT GTA | |
| AGA TAC CAA | |
| CAA TGC CAT CCT GGA | 25 |
| GTT CCT GTA AGA | |
| GCT GCC CAA TGC CAT | 26 |
| CCT GGA GTT CCT GTA | |
| AG | |
| GCC CAA TGC CAT CCT | 27 |
| GGA GTT CCT GTA AGA | |
| TAC C | |
| GCC CAA TGC CAT CCT | 28 |
| GGA GTT CCT GTA AGA | |
| T | |
| TTG CCG CTG CCC AAT | 29 |
| GCC ATC CTG GAG TTC | |
| CTG TAA GAT | |
| GCC CAA TGC CAT CCT | 30 |
| GGA GTT CCT GTA A | |
| GCC GCT GCC CAA TGC | 31 |
| CAT CCT GGA GTT CCT | |
| GTA A | |
| GCC CAA TGC CAT CCT | 32 |
| GGA GTT CCT G | |
| GCC GCT GCC CAA TGC | 33 |
| CAT CCT GGA GTT CCT | |
| G | |
| m5C-G-m5C-T-G-C-m5C- | 34 |
| m5C-A-A-T-G-m5C-m5C- | |
| A-U-m5C-m5C | |
| GAT CTG TCA AAT CGC | 35 |
| CTG CAG GTA A | |
| GAT CTG TCA AAT CGC | 36 |
| CTG CAG G | |
| CAG ATC TGT CAA ATC | 37 |
| GCC TGC AGG | |
| GAT CTG TCA AAT CGC | 38 |
| CTG CAG GT | |
| GAT CTG TCA AAT CGC | 39 |
| CTG CAG | |
| CAG ATC TGT CAA ATC | 40 |
| GCC TGC AGG T | |
| CAG ATC TGT CAA ATC | 41 |
| GCC TGC AG | |
| GGG ATC CAG TAT ACT | 42 |
| TAC AGG C | |
| GGG ATC CAG TAT ACT | 43 |
| TAC AGG CTC C | |
| GAT CCA GTA TAC TTA | 44 |
| CAG GCT CC | |
| GGA TCC AGT ATA CTT | 45 |
| ACA GGC TCC | |
| ACT TCC TCT TTA ACA | 46 |
| GAA AAG CAT AC | |
| ATC CAG TAT ACT TAC | 47 |
| AGG CTC C | |
| GAG CTC AGA TCT TCT | 48 |
| AAC TTC CTC T | |
| GGG ATC CAG TAT ACT | 49 |
| TAC AGG CTC | |
| ATG GGA TCC AGT ATA | 50 |
| CTT ACA GGC TCC | |
| CTG TTC CAA ATC CTG | 51 |
| CAT TGT TGC C | |

wherein A is

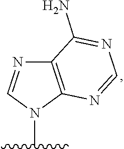 [SMILES: *n1cnc2c(N)ncnc21]

### C is

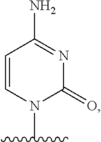 [SMILES: *n1ccc(N)nc1=O]

### G is

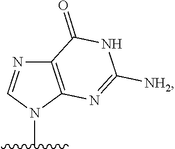 [SMILES: *n1cnc2c(=O)[nH]c(N)nc21]

### T is

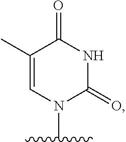 [SMILES: *n1cc(C)c(=O)[nH]c1=O]

### U is

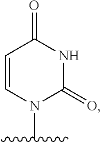 [SMILES: *n1ccc(=O)[nH]c1=O]

Gm is methylated guanine, Am is methylated adenine, and m5C is

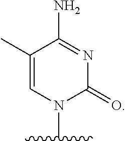 [SMILES: *n1cc(C)c(N)nc1=O]

In some embodiments, each Nu from 1 to (n+1) and 5′ to 3′ of the antisense oligonucleotide conjugate, or pharmaceutically acceptable salt thereof, of Formula (I) corresponds to SEQ ID NO: 1, SEQ ID NO: 7, or SEQ ID NO: 17.

In some embodiments, the antisense oligonucleotide conjugate is in free base form.

In some embodiments, the antisense oligonucleotide conjugate is a pharmaceutically acceptable salt.

In some embodiments, the antisense oligonucleotide conjugate is a halide salt.

In some embodiments, the antisense oligonucleotide conjugate is an HCl salt. In some embodiments, the HCl salt of the antisense oligonucleotide conjugate is a 6HCl salt.

In some embodiments, the therapeutically effective amount of the antisense oligonucleotide conjugate or pharmaceutically acceptable salt thereof is provided in a pharmaceutical composition formed by dissolving 0.005 mg/kg to about 300 mg/kg of the antisense oligonucleotide conjugate, or pharmaceutically acceptable salt thereof, in an aqueous carrier solution. In some embodiments, the therapeutically effective amount of the antisense oligonucleotide conjugate is at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg, about 0.005 mg/kg to about 200 mg/kg, about 0.1 mg/kg to about 100 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, about 80 mg/kg to about 300 mg/kg, about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg.

In some embodiments, the antisense oligonucleotide conjugate, or pharmaceutically acceptable salt thereof, is administered intravenously.

### DETAILED DESCRIPTION

Embodiments of the present invention relate to improved methods for treating diseases or disorders amenable to antisense oligonucleotide therapy by administering an effective amount of an antisense oligonucleotide or antisense oligonucleotide conjugate, or pharmaceutically acceptable salt thereof. In some embodiments, exon skipping is induced by administering an effective amount of an antisense oligonucleotide that is a phosphorodiamidate morpholino oligonucleotide (PMO) or an antisense oligonucleotide that is a PMO conjugated to a cell-penetrating peptide (PPMO), or a pharmaceutically acceptable salt thereof, which selectively binds to a target sequence. In some embodiments, the invention relates to methods of treating the disease or disorder amenable to antisense oligonucleotide therapy in which an effective amount of an antisense oligonucleotide (e.g., PMO) or antisense oligonucleotide conjugate (e.g. PPMO), or pharmaceutically acceptable salt thereof, e.g., about 0.005 mg/kg to about 300 mg/kg, at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg, about 0.005 mg/kg to about 200 mg/kg, about 0.1 mg/kg to about 100.0 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, or about 80 mg/kg to about 300 mg/kg, about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg is administered. In some embodiments, the antisense oligonucleotide (e.g., PMO) or antisense oligonucleotide conjugate (e.g., PPMO), or pharmaceutically acceptable salt thereof, is administered once every one, two, three, or four weeks. In some embodiments, the antisense oligonucleotide conjugate (e.g., PPMO), or pharmaceutically acceptable salt thereof, is administered once every four weeks. In some embodiments, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #1 dissolved in an aqueous carrier solution is administered every four weeks. In some embodiments, 20 mg/kg, 30 mg/kg, 40 mg/kg, 50 mg/kg, 60 mg/kg, 70 mg/kg, 80 mg/kg, 90 mg/kg, or 100 mg/kg of PPMO #1.6HCl dissolved in an aqueous carrier solution is administered every four weeks. In some embodiments, about 60 mg/kg of a pharmaceutically acceptable salt of PPMO #1 dissolved in an aqueous carrier solution is administered every four weeks. In some embodiments, about 80 mg/kg of a pharmaceutically acceptable salt of PPMO #1 dissolved in an aqueous carrier solution is administered every four weeks. In some embodiments, about 100 mg/kg of a pharmaceutically acceptable salt of PPMO #1 dissolved in an aqueous carrier solution is administered every four weeks. In some embodiments, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #2 dissolved in an aqueous carrier solution is administered every four weeks. In some embodiments, the about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #3 dissolved in an aqueous carrier solution is administered every four weeks. In some embodiments, the antisense oligonucleotide (e.g., PMO) or antisense oligonucleotide conjugate (e.g., PPMO), or pharmaceutically acceptable salt thereof, is administered monthly.

Examples of such diseases or disorders amenable to antisense oligonucleotide therapy include muscular dystrophy, such as DMD and BMD, by administering antisense compounds that are specifically designed to induce exon skipping in the human dystrophin gene. Dystrophin plays a vital role in muscle function, and various muscle-related diseases are characterized by mutated forms of this gene. Hence, in certain embodiments, the improved methods described herein may be used for inducing exon skipping in mutated forms of the human dystrophin gene, such as the mutated dystrophin genes found in DMD and BMD.

Due to aberrant mRNA splicing events caused by mutations, these mutated human dystrophin genes either express defective dystrophin protein or express no measurable dystrophin at all, a condition that leads to various forms of muscular dystrophy. To remedy this condition, the antisense oligonucleotide or antisense oligonucleotide conjugate hybridizes to selected regions of a pre-processed RNA of a mutated human dystrophin gene, induce exon skipping and differential splicing in that otherwise aberrantly spliced dystrophin mRNA, and thereby allow muscle cells to produce an mRNA transcript that encodes a functional dystrophin protein. In certain embodiments, the resulting dystrophin protein is not necessarily the “wild-type” form of dystrophin, but is rather a truncated, yet functional or semi-functional, form of dystrophin.

By increasing the levels of functional dystrophin protein in muscle cells, these and related embodiments are useful in the prophylaxis and treatment of muscular dystrophy, especially those forms of muscular dystrophy, such as DMD and BMD, that are characterized by the expression of defective dystrophin proteins due to aberrant mRNA splicing. The methods described herein further provide improved treatment options for patients with muscular dystrophy and offer significant and practical advantages over alternate methods of treating relevant forms of muscular dystrophy. For example, in some embodiments, the methods relate to the administration of an antisense compound for inducing exon skipping in the human dystrophin gene for a longer duration than prior approaches.

Thus, in one embodiment, the invention relates to methods for treating muscular dystrophy such as DMD and BMD, by inducing exon skipping in a human patient. In some embodiments, exon skipping is induced by administering an effective amount of an antisense oligonucleotide that is a phosphorodiamidate morpholino oligonucleotide (PMO) or an antisense oligonucleotide that is a PMO conjugated to a cell-penetrating peptide (PPMO), or a pharmaceutically acceptable salt thereof, which selectively binds to a target sequence in an exon of dystrophin pre-mRNA. In some embodiments, the invention relates to methods of treating DMD or BMD in which an effective amount of an antisense oligonucleotide (e.g., PMO) or antisense oligonucleotide conjugate (e.g., PPMO), or pharmaceutically acceptable salt thereof, e.g., about 0.005 mg/kg to about 300 mg/kg, at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg, about 0.005 mg/kg to about 200 mg/kg, about 0.1 mg/kg to about 100 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, about 80 mg/kg to about 300 mg/kg, about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg is administered. In some embodiments, the antisense oligonucleotide (e.g., PMO) or antisense oligonucleotide conjugate (e.g., PPMO), or pharmaceutically acceptable salt thereof, is administered once every one, two, three, or four weeks. In some embodiments, the antisense oligonucleotide conjugate (e.g., PPMO), or pharmaceutically acceptable salt thereof, is administered once every four weeks. In some embodiments, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #1 dissolved in an aqueous carrier solution is administered every four weeks. In some embodiments, 20 mg/kg, 30 mg/kg, 40 mg/kg, 50 mg/kg, 60 mg/kg, 70 mg/kg, 80 mg/kg, 90 mg/kg, or 100 mg/kg of PPMO #1.6HCl dissolved in an aqueous carrier solution is administered every four weeks. In some embodiments, about 60 mg/kg of a pharmaceutically acceptable salt of PPMO #1 dissolved in an aqueous carrier solution is administered every four weeks. In some embodiments, about 80 mg/kg of a pharmaceutically acceptable salt of PPMO #1 dissolved in an aqueous carrier solution is administered every four weeks. In some embodiments, about 100 mg/kg of a pharmaceutically acceptable salt of PPMO #1 dissolved in an aqueous carrier solution is administered every four weeks. In some embodiments, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #2 dissolved in an aqueous carrier solution is administered every four weeks. In some embodiments, the about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #3 dissolved in an aqueous carrier solution is administered every four weeks. In some embodiments, the antisense oligonucleotide (e.g., PMO) or antisense oligonucleotide conjugate (e.g., PPMO), or pharmaceutically acceptable salt thereof, is administered monthly.

### I. DEFINITIONS

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B. or C; A or C; A or B; B or C; A and C; A and B; B and C; A (alone); B (alone); and C (alone).

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The terms “complementary” and “complementarity” refer to polynucleotides (i.e., a sequence of nucleotides) related by base-pairing rules. For example, the sequence “T-G-A (5′-3′),” is complementary to the sequence “T-C-A (5′-3′).” Complementarity may be “partial,” in which only some of the nucleic acids' bases are matched according to base pairing rules. Or, there may be “complete” or “total” complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5′ and/or 3′ terminus.

The terms “antisense oligomer,” “antisense compound,” and “antisense oligonucleotide” are used interchangeably and refer to a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. The cyclic subunits are based on ribose or another pentose sugar or, in a preferred embodiment, a morpholino group (see description of morpholino oligomers below). The oligomer may have exact or near sequence complementarity to the target sequence; variations in sequence near the termini of an oligomer are generally preferable to variations in the interior.

Such an antisense oligomer can be designed to block or inhibit translation of mRNA or to inhibit natural pre-mRNA splice processing, and may be said to be “directed to” or “targeted against” a target sequence with which it hybridizes. The target sequence is typically a region including an AUG start codon of an mRNA, a Translation Suppressing Oligomer, or splice site of a pre-processed mRNA, a Splice Suppressing Oligomer (SSO). The target sequence for a splice site may include an mRNA sequence having its 5′ end 1 to about 25 base pairs downstream of a normal splice acceptor junction in a preprocessed mRNA. A preferred target sequence is any region of a preprocessed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above.

The terms “antisense oligomer conjugate” and “antisense oligonucleotide conjugate” are used interchangeably and refer to an antisense oligonucleotide conjugated to a cell-penetrating peptide.

The terms “cell penetrating peptide” and “CPP” are used interchangeably and refer to cationic cell penetrating peptides, also called transport peptides, carrier peptides, or peptide transduction domains. The peptides, as shown herein, have the capability of inducing cell penetration within 100% of cells of a given cell culture population and allow macromolecular translocation within multiple tissues in vivo upon systemic administration. A preferred CPP embodiment is an arginine-rich peptide as described further below.

The terms “morpholino,” “morpholino oligomer,” and “PMO” refer to a phosphorodiamidate morpholino oligomer of the following general structure:

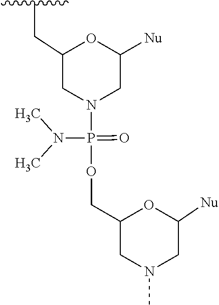

and as described in FIG. 2 of Summerton, J., et al., Antisense & Nucleic Acid Drug Development, 7: 187-195 (1997). Morpholinos as described herein include all stereoisomers and tautomers of the foregoing general structure. The synthesis, structures, and binding characteristics of morpholino oligomers are detailed in U.S. Pat. Nos. 5,698,685; 5,217,866; 5,142,047; 5,034,506; 5,166,315; 5,521,063; 5,506,337; 8,076,476; and 8,299,206; each of which is incorporated by reference herein in its entirety.

In certain embodiments, a morpholino is conjugated at the 5′ or 3′ end of the oligomer with a “tail” moiety to increase its stability and/or solubility. Exemplary tails include:

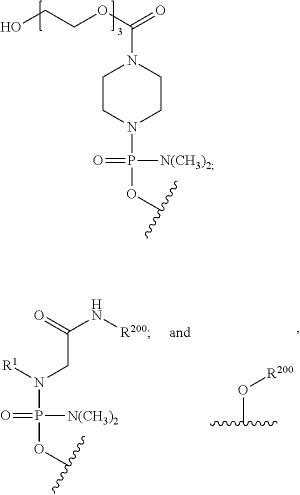

wherein R²⁰⁰ is hydrogen or a cell-penetrating peptide R¹ is C₁-C₆ alkyl.

In one embodiment, an exemplary tail moiety, “TEG” or “EG3” refers to the following tail moiety:

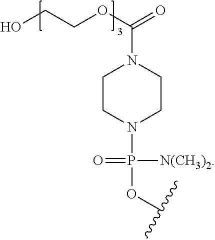

In one embodiment, an exemplary tail moiety, “GT” refers to the following tail moiety:

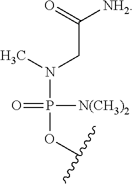 [SMILES: *OP(=O)(N(C)C)N(C)CC(N)=O]

As used herein, the terms “-G-R₅” (SEQ ID NO: 59) and “-G-R₅—Ac” (SEQ ID NO: 59) are used interchangeably and refer to a peptide moiety conjugated to an antisense oligonucleotide of the disclosure. In various embodiments, “G” represents a glycine residue conjugated to “R₅” (SEQ ID NO: 60) by an amide bond, and each “R” represents an arginine residue conjugated together by amide bonds such that “R₅” means five (5) arginine residues (SEQ ID NO: 60) conjugated together by amide bonds. The arginine residues can have any stereo configuration, for example, the arginine residues can be L-arginine residues, D-arginine residues, or a mixture of D- and L-arginine residues. In certain embodiments, “-G-R₅” (SEQ ID NO: 59) or “-G-R₅—Ac” (SEQ ID NO: 59) is linked to the distal —OH or NH₂ of the “tail” moiety. In certain embodiments, “-G-R₅” (SEQ ID NO: 59) or “-G-R₅—Ac” (SEQ ID NO: 59) is conjugated to the morpholine ring nitrogen of the 3′ most morpholino subunit of a PMO antisense oligonucleotide of the disclosure. In some embodiments, “-G-R₅” (SEQ ID NO: 59) or “-G-R₅—Ac” (SEQ ID NO: 59) is conjugated to the 3′ end of an antisense oligonucleotide of the disclosure and is of the following formula:

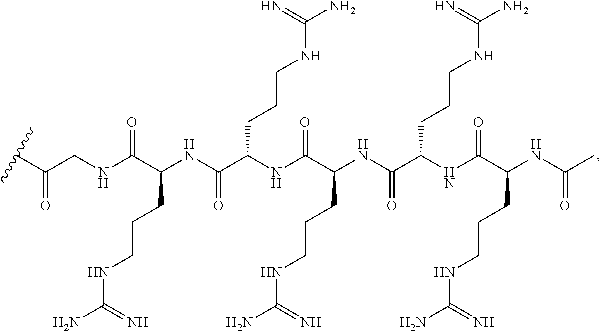 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O]

or a pharmaceutically acceptable salt thereof, or

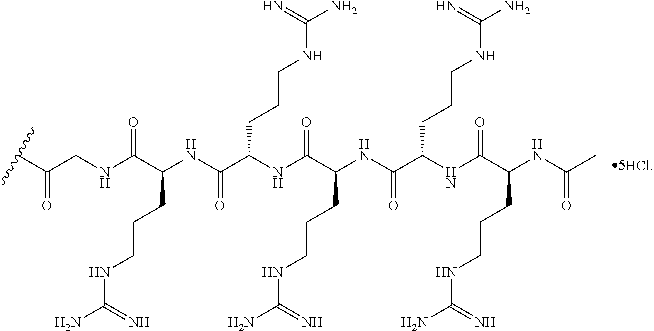 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O]

As used herein, the terms “-G-R₆” (SEQ ID NO: 57) and “-G-R₆—Ac” (SEQ ID NO: 57) and “R₆G” (SEQ ID NO: 57) are used interchangeably and refer to a peptide moiety conjugated to an antisense oligonucleotide of the disclosure. In various embodiments, “G” represents a glycine residue conjugated to “R₆” (SEQ ID NO: 58) by an amide bond, and each “R” represents an arginine residue conjugated together by amide bonds such that “R₆” means six (6) arginine residues (SEQ ID NO: 58) conjugated together by amide bonds. The arginine residues can have any stereo configuration, for example, the arginine residues can be L-arginine residues, D-arginine residues, or a mixture of D- and L-arginine residues. In certain embodiments, “-G-R₆” (SEQ ID NO: 57) or “-G-R₆—Ac” (SEQ ID NO: 57) is linked to the distal —OH or —NH₂ of the “tail” moiety. In certain embodiments, “-G-R₆” (SEQ ID NO: 57) or “-G-R₆—Ac” (SEQ ID NO: 57) is conjugated to the morpholine ring nitrogen of the 3′ most morpholino subunit of a PMO antisense oligonucleotide of the disclosure. In some embodiments, “-G-R₆” (SEQ ID NO: 57) or “-G-R₆—Ac” (SEQ ID NO: 57) is conjugated to the 3′ end of an antisense oligonucleotide of the disclosure and is of the following formula:

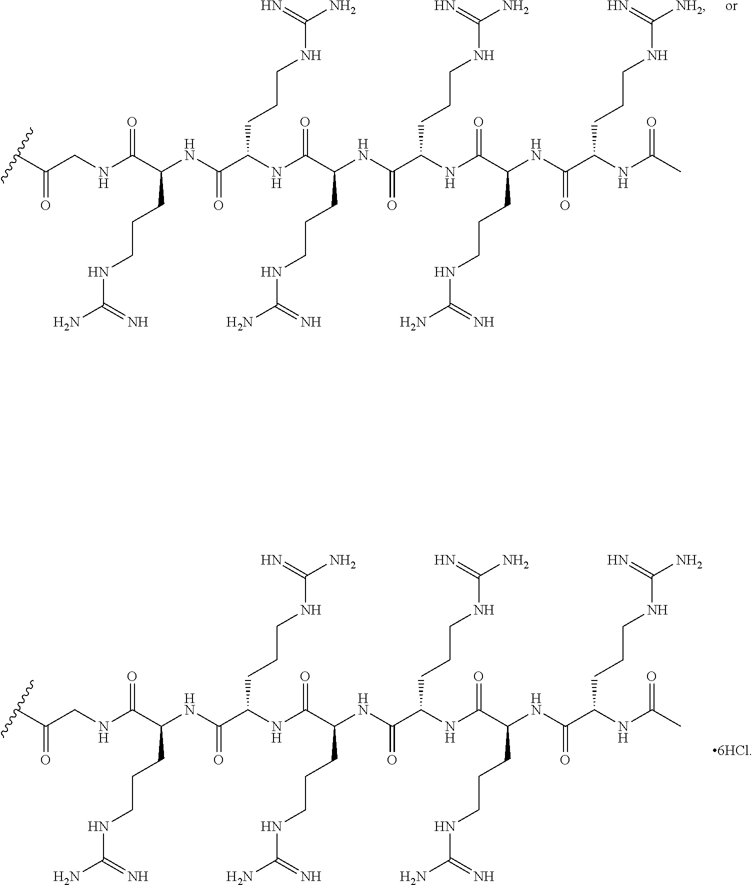 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O ; *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O]

The terms “nucleobase” (Nu), “base pairing moiety” or “base” are used interchangeably to refer to a purine or pyrimidine base found in naturally occurring, or “native” DNA or RNA (e.g., uracil, thymine, adenine, cytosine, and guanine), as well as analogs of these naturally occurring purines and pyrimidines. These analogs may confer improved properties, such as binding affinity, to the oligomer. Exemplary analogs include hypoxanthine (the base component of inosine); 2,6-diaminopurine; 5-methyl cytosine; C5-propynyl-modified pyrimidines; 10-(9-(aminoethoxy)phenoxazinyl) (G-clamp); methyl adenine (“Am”); methyl guanine (“Gm”); and the like.

Further examples of base pairing moieties include, but are not limited to, uracil, thymine, adenine, cytosine, guanine and hypoxanthine (inosine) having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in: Chiu and Rana, R N A, 2003, 9, 1034-1048; Limbach et al. Nucleic Acids Research, 1994, 22, 2183-2196; and Revankar and Rao, Comprehensive Natural Products Chemistry, vol. 7, 313; are also contemplated, the contents of which are incorporated herein by reference.

Further examples of base pairing moieties include, but are not limited to, expanded-size nucleobases in which one or more benzene rings has been added. Nucleic acid base replacements described in: the Glen Research catalog (www.glenresearch.com); Krueger A T et al., Acc. Chem. Res., 2007, 40, 141-150; Kool, E T, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; and Hirao, I., Curr. Opin. Chem. Biol., 2006, 10, 622-627; the contents of which are incorporated herein by reference, are contemplated as useful in the antisense oligomers described herein. Examples of expanded-size nucleobases include those shown below, as well as tautomeric forms thereof.

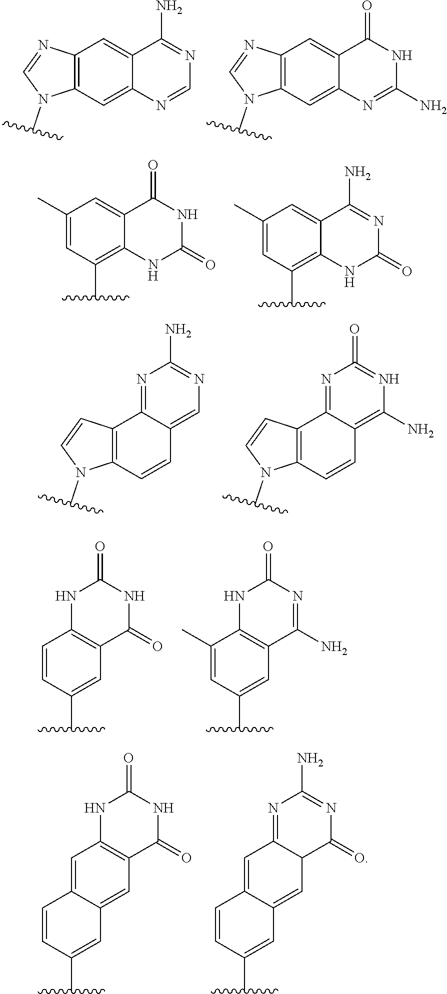

The term “PPMO” refers to PMO conjugated to a cell-penetrating peptide.

“Eteplirsen”, also known as “AVN-4658” is a PMO having the base sequence 5′-CTCCAACATCAAGGAAGATGGCATTTCTAG-3′ (SEQ ID NO:1). Eteplirsen is registered under CAS Registry Number 1173755-55-9. Chemical names include: RNA, [P-deoxy-P-(dimethylamino)] (2′,3′-dideoxy-2′,3′-imino-2′,3′-seco) (2′a→5′) (C-m5U-C-C-A-A-C-A-m5U-C-A-A-G-G-A-A-G-A-m5U-G-G-C-A-m5U-m5U-m5U-C-m5U-A-G) (SEQ ID NO: 61), 5′-[P-[4-[[2-[2-(2-hydroxyethoxy)ethoxy]ethoxy]carbonyl]-1-piperazinyl]-N,N-dimethylphosphonamidate]and P,2′,3′-trideoxy-P-(dimethylamino)-5′-O—{P-[4-(10-hydroxy-2,5,8-trioxadecanoyl)piperazin-1-yl]-N,N-dimethylphosphonamidoyl}-2′,3′-imino-2′,3′-secocytidylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoadenylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,3′-dideoxy-P-(dimethylamino)-2′,3′-imino-2′,3′-secothymidylyl-(2′a→5′)-P,3′-dideoxy-P-(dimethylamino)-2′,3′-imino-2′,3′-secothymidylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secoadenylyl-(2′a→5′)-2′,3′-dideoxy-2′,3′-imino-2′,3′-secoguanosine

Eteplirsen has the following structure (SEQ ID NO: 1 disclosed below):

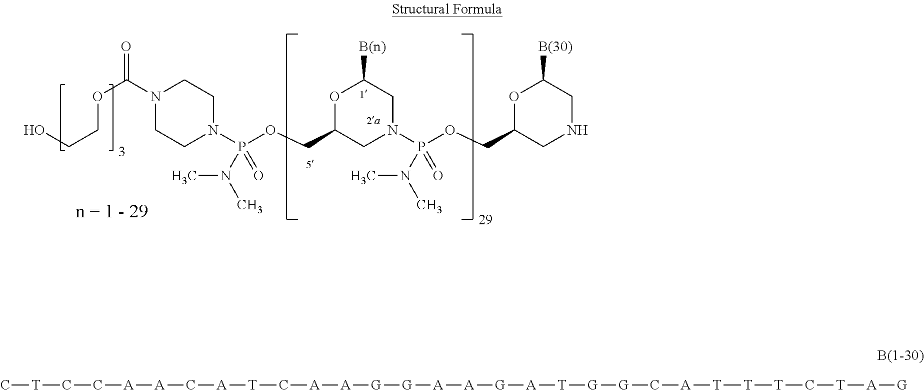

“PPMO #1” has the following structure:

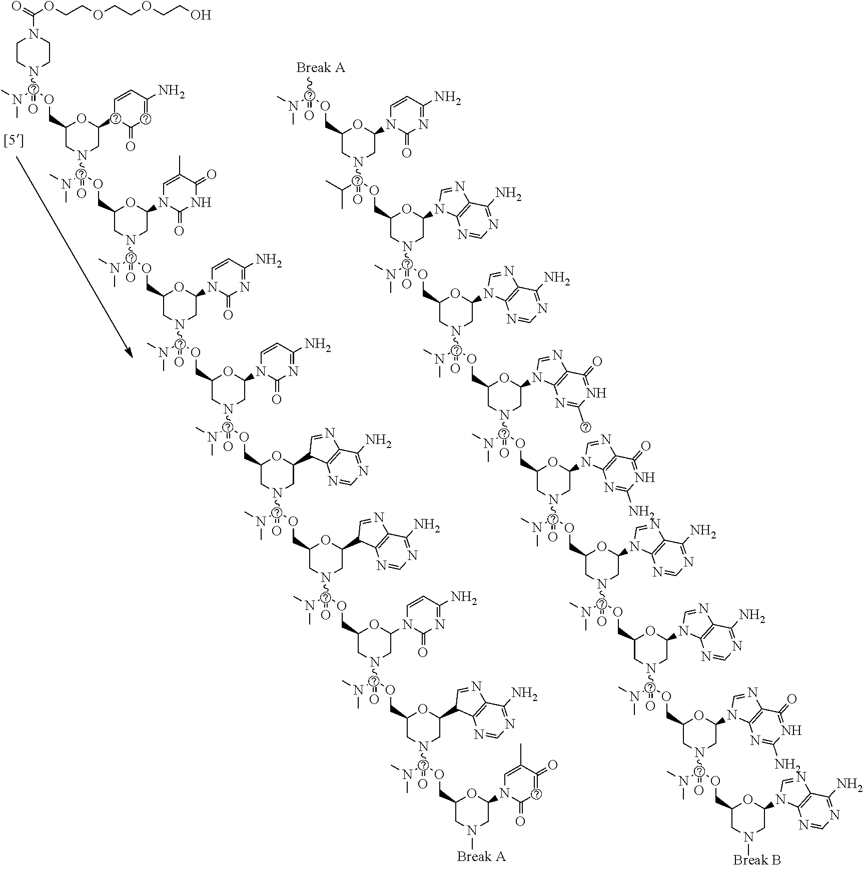

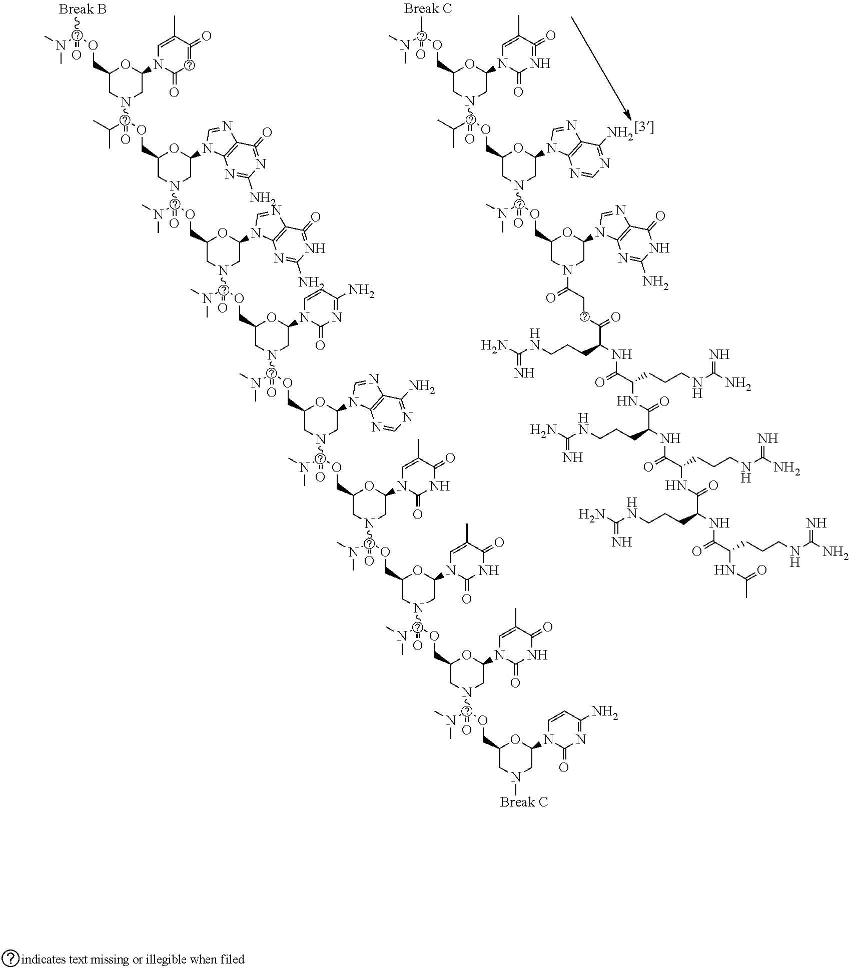

or a pharmaceutically acceptable salt thereof. In some embodiments, PPMO #1 is in the form of a halide salt. In some embodiments, PPMO #1 is in the form of a hexahalide salt form. In some embodiments, PPMO #1 is in the form of an HCl (hydrochloric acid) salt. In certain embodiments, the HCl salt is a .6HCl salt.

“Golodirsen,” also known by its code name “SRP-4053” is a PMO having the base sequence 5′-GTTGCCTCCGGTTCTGAAGGTGTTC-3′ (SEQ ID NO:7). Golodirsen is registered under CAS Registry Number 1422959-91-8. Chemical names include: all-P-ambo-[P,2′,3′-trideoxy-P-(dimethylamino)-2′,3′-imino-2′,3′-seco](2′a-→5′)(G-T-T-G-C-C-T-C-C-G-G-T-T-C-T-G-A-A-G-G-T-G-T-T-C) (SEQ ID NO: 7) 5′-[4-({2-[2-(2-hydroxyethoxy)ethoxy]ethoxy}carbonyl)-N,N-dimethylpiperazine-1-phosphonamidate]

Golodirsen has the following structure (SEQ ID NO: 7 disclosed below):

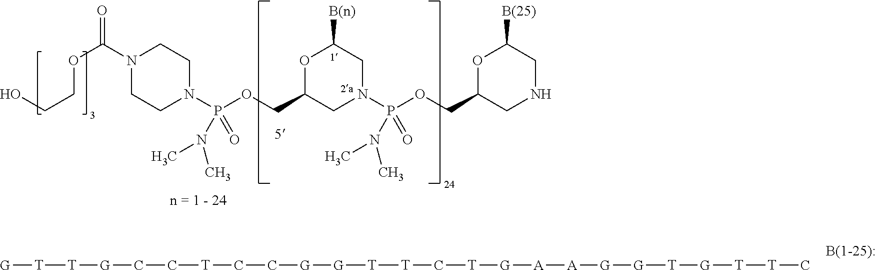

“PPMO #2” is has the following structure:

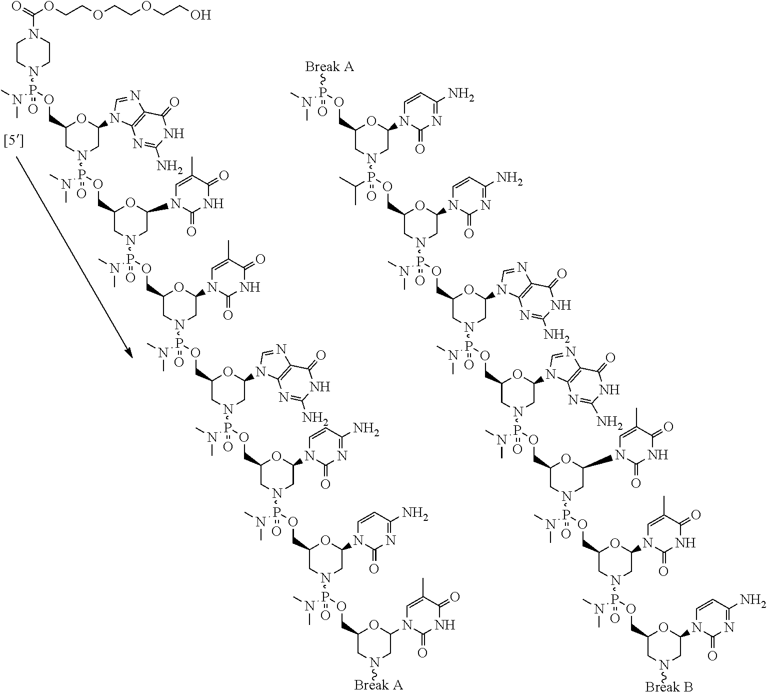

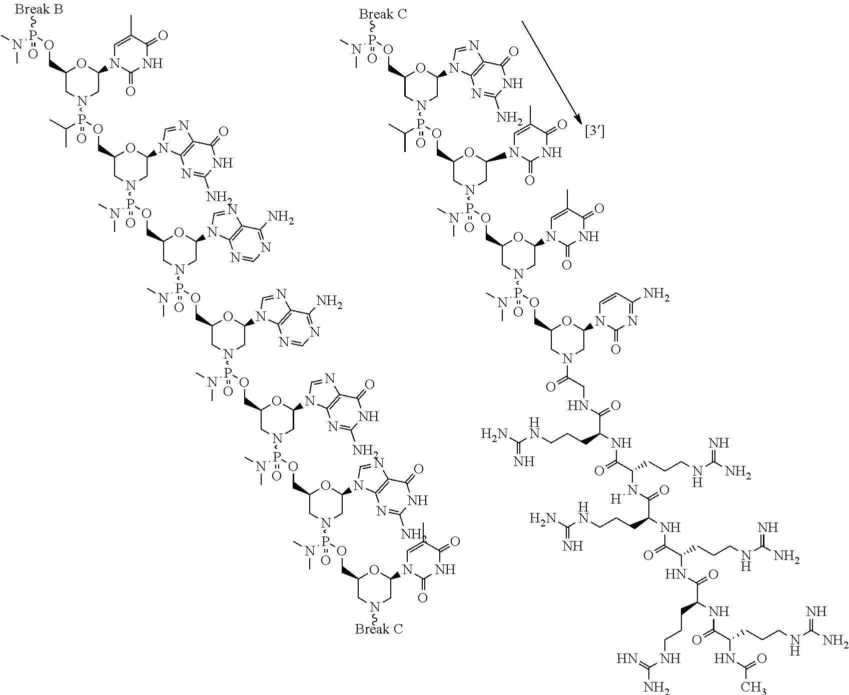

or a pharmaceutically acceptable salt thereof. In some embodiments, PPMO #2 is in the form of a halide salt. In some embodiments, PPMO #2 is in the form of a hexahalide salt form. In some embodiments, PPMO #2 is in the form of an HCl (hydrochloric acid) salt. In certain embodiments, the HCl salt is a .6HCl salt.

“Casimersen” also known by its code name “SRP-4045” is a PMO having the base sequence 5′-CAATGCCATCCTGGAGTTCCTG-3′ (SEQ ID NO:17). Casimersen is registered under CAS Registry Number 1422959-91-8. Chemical names include: all-P-ambo-[P,2′,3′-trideoxy-P-(dimethylamino)-2′,3′-imino-2′,3′-seco](2′a→5′)(C-A-A-T-G-C-C-A-T-C-C-T-G-G-A-G-T-T-C-C-T-G) SEQ ID NO: 17) 5′-[4-({2-[2-(2-hydroxyethoxy)ethoxy]ethoxy}carbonyl)-N,N-dimethylpiperazine-1-phosphonamidate]

Casimersen has the following chemical structure (SEQ ID NO: 17 disclosed below):

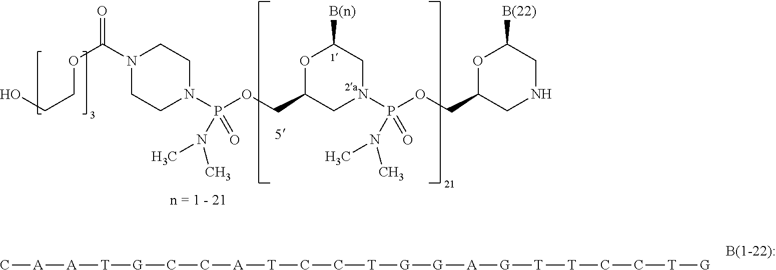

“PPMO #3” is has the following structure:

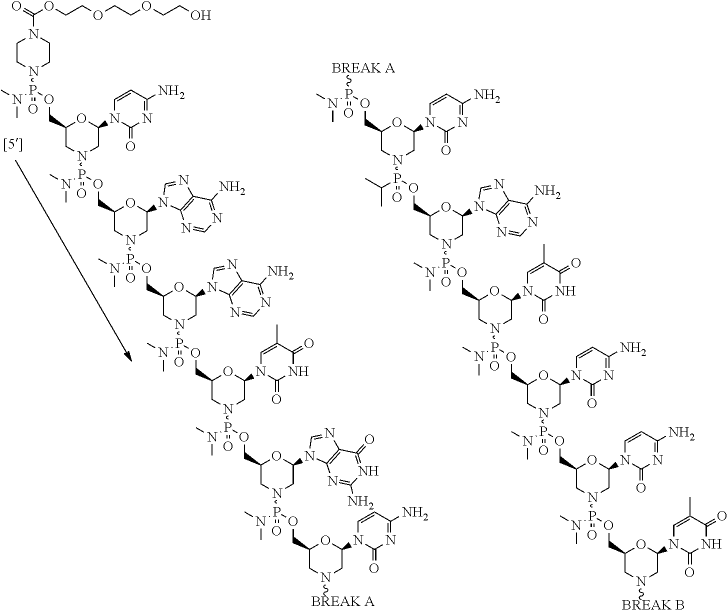

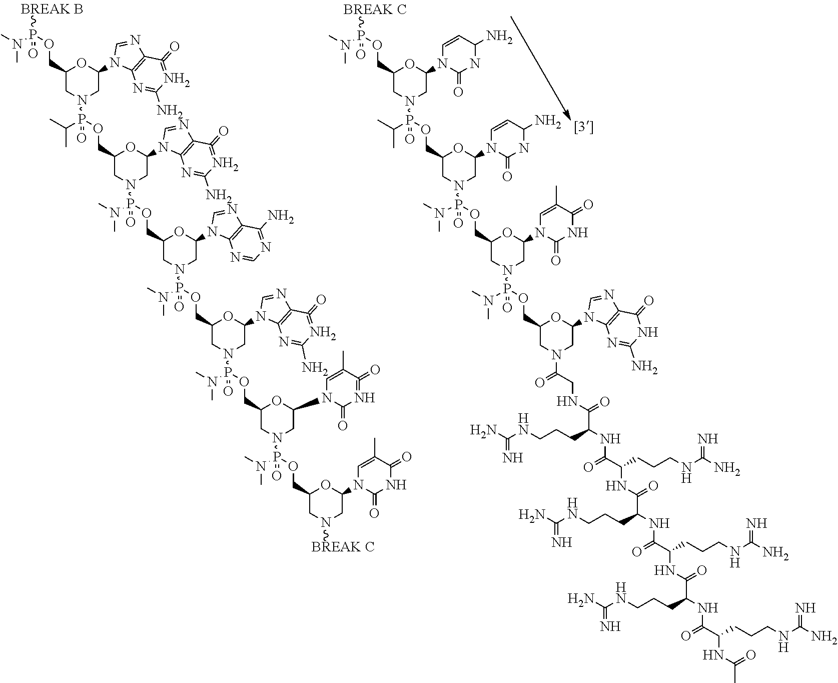

or a pharmaceutically acceptable salt thereof. In some embodiments, PPMO #3 is in the form of a halide salt. In some embodiments, PPMO #3 is in the form of a hexahalide salt form. In some embodiments, PPMO #3 is in the form of an HCl (hydrochloric acid) salt. In certain embodiments, the HCl salt is a .6HCl salt.

An “amino acid subunit” or “amino acid residue” can refer to an ca-amino acid residue (—CO—CHR¹⁰—NH—) or a β- or other amino acid residue (e.g., —CO—(CH₂)ₙCHR¹⁰—NH—), where R¹⁰ is a side chain (which may include hydrogen) and n is 1 to 6, preferably 1 to 4.

The term “naturally occurring amino acid” refers to an amino acid present in proteins found in nature. The term “non-natural amino acids” refers to those amino acids not present in proteins found in nature, examples include beta-alanine (β-Ala), 6-aminohexanoic acid (Ahx) and 6-aminopentanoic acid.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. The human dystrophin gene has about 79 exons.

An “intron” refers to a nucleic acid region (within a gene) that is not translated into a protein. An intron is a non-coding section that is transcribed into a precursor mRNA (pre-mRNA), and subsequently removed by splicing during formation of the mature RNA.

An “effective amount” or “therapeutically effective amount” refers to an amount of therapeutic compound, such as an antisense oligonucleotide or antisense oligonucleotide conjugate, or pharmaceutically acceptable salt thereof, administered to a human patient, either as a single dose or as part of a series of doses, which is effective to produce a desired therapeutic effect. For an antisense oligonucleotide or an antisense oligonucleotide conjugate, this effect is typically brought about by inhibiting translation or natural splice-processing of a selected target sequence. In some embodiments, an effective amount is at least 0.05 mg/kg of an antisense oligonucleotide or an antisense oligonucleotide conjugate, or pharmaceutically acceptable salt thereof. In another embodiment, an effective amount is an amount from about 0.005 mg/kg to about 300 mg/kg. In some embodiments, the therapeutically effective amount is at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg. In some embodiments, the therapeutically effective amount is about 0.005 mg/kg to about 200 mg/kg, about 0.1 mg/kg to about 100 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, or about 80 mg/kg to about 300 mg/kg. In some embodiments, the therapeutically effective amount is about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg.

“Exon skipping” refers generally to the process by which an entire exon, or a portion thereof, is removed from a given pre-processed RNA, and is thereby excluded from being present in the mature RNA, such as the mature mRNA that is translated into a protein. Hence, the portion of the protein that is otherwise encoded by the skipped exon is not present in the expressed form of the protein, typically creating an altered, though still functional, form of the protein. In certain embodiments, the exon being skipped is an aberrant exon from the human dystrophin gene, which may contain a mutation or other alteration in its sequence that otherwise causes aberrant splicing. In certain embodiments, the exon being skipped is exon 44, 45, 50, 51, 52, or 53 of the human dystrophin gene.

“Dystrophin” is a rod-shaped cytoplasmic protein, and a vital part of the protein complex that connects the cytoskeleton of a muscle fiber to the surrounding extracellular matrix through the cell membrane. Dystrophin contains multiple functional domains. For instance, dystrophin contains an actin binding domain at about amino acids 14-240 and a central rod domain at about amino acids 253-3040. This large central domain is formed by 24 spectrin-like triple-helical elements of about 109 amino acids, which have homology to alpha-actinin and spectrin. The repeats are typically interrupted by four proline-rich non-repeat segments, also referred to as hinge regions. Repeats 15 and 16 are separated by an 18 amino acid stretch that appears to provide a major site for proteolytic cleavage of dystrophin. The sequence identity between most repeats ranges from 10-25%. One repeat contains three alpha-helices: 1, 2 and 3. Alpha-helices 1 and 3 are each formed by 7 helix turns, probably interacting as a coiled-coil through a hydrophobic interface. Alpha-helix 2 has a more complex structure and is formed by segments of four and three helix turns, separated by a Glycine or Proline residue. Each repeat is encoded by two exons, typically interrupted by an intron between amino acids 47 and 48 in the first part of alpha-helix 2. The other intron is found at different positions in the repeat, usually scattered over helix-3. Dystrophin also contains a cysteine-rich domain at about amino acids 3080-3360), including a cysteine-rich segment (i.e., 15 Cysteines in 280 amino acids) showing homology to the C-terminal domain of the slime mold (Dictyostelium discoideum) alpha-actinin. The carboxy-terminal domain is at about amino acids 3361-3685.

The amino-terminus of dystrophin binds to F-actin and the carboxy-terminus binds to the dystrophin-associated protein complex (DAPC) at the sarcolemma. The DAPC includes the dystroglycans, sarcoglycans, integrins and caveolin, and mutations in any of these components cause autosomally inherited muscular dystrophies. The DAPC is destabilized when dystrophin is absent, which results in diminished levels of the member proteins, and in turn leads to progressive fibre damage and membrane leakage. In various forms of muscular dystrophy, such as Duchenne's muscular dystrophy (DMD) and Becker's muscular dystrophy (BMD), muscle cells produce an altered and functionally defective form of dystrophin, or no dystrophin at all, mainly due to mutations in the gene sequence that lead to incorrect splicing. The predominant expression of the defective dystrophin protein, or the complete lack of dystrophin or a dystrophin-like protein, leads to rapid progression of muscle degeneration, as noted above. In this regard, a “defective” dystrophin protein may be characterized by the forms of dystrophin that are produced in certain patients with DMD or BMD, as known in the art, or by the absence of detectable dystrophin.

As used herein, the terms “function” and “functional” and the like refer to a biological, enzymatic, or therapeutic function.

A “functional” dystrophin protein refers generally to a dystrophin protein having sufficient biological activity to reduce the progressive degradation of muscle tissue that is otherwise characteristic of muscular dystrophy, typically as compared to the altered or “defective” form of dystrophin protein that is present in certain patients with DMD or BMD. In certain embodiments, a functional dystrophin protein may have about 10%, 20%, 30%, 40%, 50%, 60%, 70%, 80%, 90%, or 100% (including all integers in between) of the in vitro or in vivo biological activity of wild-type dystrophin, as measured according to routine techniques in the art. As one example, dystrophin-related activity in muscle cultures in vitro can be measured according to myotube size, myofibril organization (or disorganization), contractile activity, and spontaneous clustering of acetylcholine receptors. Animal models are also valuable resources for studying the pathogenesis of disease, and provide a means to test dystrophin-related activity. Two of the most widely used animal models for DMD research are the mdx mouse and the golden retriever muscular dystrophy (GRMD) dog, both of which are dystrophin negative. These and other animal models can be used to measure the functional activity of various dystrophin proteins. Included are truncated forms of dystrophin, such as those forms that are produced by certain of the exon-skipping antisense oligonucleotides or antisense oligonucleotide conjugates.

The term “restoration” of dystrophin synthesis or production refers generally to the production of a dystrophin protein including truncated forms of dystrophin in a human patient with muscular dystrophy following treatment with an antisense oligonucleotide or antisense oligonucleotide conjugate, or pharmaceutically acceptable salt thereof, as described herein. The percent of dystrophin-positive fibers in a human patient following treatment can be determined by a muscle biopsy using known techniques. For example, a muscle biopsy may be taken from a suitable muscle, such as the biceps brachii muscle in a human patient.

Analysis of the percentage of positive dystrophin fibers may be performed pre-treatment and/or post-treatment or at time points throughout the course of treatment. In some embodiments, a post-treatment biopsy is taken from the contralateral muscle from the pre-treatment biopsy. Pre- and post-treatment dystrophin expression studies may be performed using any suitable assay for dystrophin. In one embodiment, immunohistochemical detection is performed on tissue sections from the muscle biopsy using an antibody that is a marker for dystrophin, such as a monoclonal or a polyclonal antibody. For example, the MANDYS106 antibody can be used which is a highly sensitive marker for dystrophin. Any suitable secondary antibody may be used.

In some embodiments, the percent dystrophin-positive fibers are calculated by dividing the number of positive fibers by the total fibers counted. Normal muscle samples have 100% dystrophin-positive fibers. Therefore, the percent dystrophin-positive fibers can be expressed as a percentage of normal. To control for the presence of trace levels of dystrophin in the pretreatment muscle as well as revertant fibers a baseline can be set using sections of pre-treatment muscles from each patient when counting dystrophin-positive fibers in post-treatment muscles. This may be used as a threshold for counting dystrophin-positive fibers in sections of post-treatment muscle in that patient. In other embodiments, antibody-stained tissue sections can also be used for dystrophin quantification using Bioquant image analysis software (Bioquant Image Analysis Corporation, Nashville, Tenn.). The total dystrophin fluorescence signal intensity can be reported as a percentage of normal. In addition, Western blot analysis with monoclonal or polyclonal anti-dystrophin antibodies can be used to determine the percentage of dystrophin positive fibers. For example, the anti-dystrophin antibody NCL-Dys1 from Novacastra may be used. The percentage of dystrophin-positive fibers can also be analyzed by determining the expression of the components of the sarcoglycan complex (β,γ) and/or neuronal NOS.

In some embodiments, treatment with an antisense oligonucleotide or antisense oligonucleotide conjugate slows or reduces the progressive respiratory muscle dysfunction and/or failure in patients with DMD that would be expected without treatment. In one embodiment, treatment with an antisense oligonucleotide or antisense oligonucleotide conjugate may reduce or eliminate the need for ventilation assistance that would be expected without treatment. In one embodiment, measurements of respiratory function for tracking the course of the disease, as well as the evaluation of potential therapeutic interventions include Maximum inspiratory pressure (MIP), maximum expiratory pressure (MEP) and forced vital capacity (FVC). MIP and MEP measure the level of pressure a person can generate during inhalation and exhalation, respectively, and are sensitive measures of respiratory muscle strength. MIP is a measure of diaphragm muscle weakness.

In one embodiment, MEP may decline before changes in other pulmonary function tests, including MIP and FVC. In another embodiment, MEP may be an early indicator of respiratory dysfunction. In another embodiment, FVC may be used to measure the total volume of air expelled during forced exhalation after maximum inspiration. In patients with DMD, FVC increases concomitantly with physical growth until the early teens. However, as growth slows or is stunted by disease progression, and muscle weakness progresses, the vital capacity enters a descending phase and declines at an average rate of about 8 to 8.5 percent per year after 10 to 12 years of age. In another embodiment, MIP percent predicted (MIP adjusted for weight), MEP percent predicted (MEP adjusted for age) and FVC percent predicted (FVC adjusted for age and height) are supportive analyses.

By “isolated” is meant material that is substantially or essentially free from components that normally accompany it in its native state. For example, an “isolated polynucleotide,” as used herein, may refer to a polynucleotide that has been purified or removed from the sequences that flank it in a naturally-occurring state, e.g., a DNA fragment that has been removed from the sequences that are normally adjacent to the fragment.

As used herein, “sufficient length” refers to an antisense oligonucleotide or antisense oligonucleotide conjugate that is complementary to at least 8, more typically 8-30, contiguous nucleobases in a target dystrophin pre-mRNA. In some embodiments, an antisense of sufficient length includes at least 8, 9, 10, 11, 12, 13, 14, or 15 contiguous nucleobases in the target dystrophin pre-mRNA. In other embodiments an antisense of sufficient length includes at least 16, 17, 18, 19, 20, 21, 22, 23, 24, or 25 contiguous nucleobases in the target dystrophin pre-mRNA. An antisense oligonucleotide or antisense oligonucleotide conjugate of sufficient length has at least a minimal number of nucleotides to be capable of specifically hybridizing to any one or more of exons 1-79 of the dystrophin gene. Preferably, the antisense oligonucleotide or antisense oligonucleotide conjugate has a minimal number of nucleotides to be capable of specifically hybridizing to and induce skipping of one or more exons. In one embodiment, the antisense oligonucleotide or antisense oligonucleotide conjugate has a minimal number of nucleotides to be capable of specifically hybridizing to and induce skipping of any one or more of exons 44, 45, 50, 51, 52, or 53 of the human dystrophin gene.

By “enhance” or “enhancing,” or “increase” or “increasing,” or “stimulate” or “stimulating,” refers generally to the ability of one or antisense oligonucleotide or antisense oligonucleotide conjugate to produce or cause a greater physiological response (i.e., downstream effects) in a cell or a patient, as compared to the response caused by either no antisense oligonucleotide or antisense oligonucleotide conjugate or a control compound. A measurable physiological response may include increased expression of a functional form of a dystrophin protein, or increased dystrophin-related biological activity in muscle tissue, among other responses apparent from the understanding in the art and the description herein. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7, 1.8, etc.) the amount produced by no antisense compound (the absence of an agent) or a control compound.

The term “reduce” or “inhibit” may relate generally to the ability of one or more antisense oligonucleotides or antisense oligonucleotide conjugates to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of the particular disease or disorder being treated. For example, relevant physiological or cellular responses include reductions in the symptoms or pathology of muscular dystrophy, or reductions in the expression of defective forms of dystrophin, such as the altered forms of dystrophin that are expressed in individuals with DMD or BMD.

“Treatment” of an individual (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or disorder. For example, treatment can include any desirable effect on the symptoms or pathology of a disease or disorder associated with the dystrophin protein, as in certain forms of muscular dystrophy, and may include, for example, minimal changes or improvements in one or more measurable markers of the disease or condition being treated. Also included are “prophylactic” treatments, which can be directed to reducing the rate of progression of the disease or disorder being treated, delaying the onset of that disease or disorder, or reducing the severity of its onset. “Treatment” or “prophylaxis” does not necessarily indicate complete eradication, cure, or prevention of the disease or condition, or associated symptoms thereof.

In one embodiment, treatment with an antisense oligonucleotide or antisense oligonucleotide conjugate, or pharmaceutically acceptable salt thereof, increases novel dystrophin production and slows or reduces the loss of ambulation that would be expected without treatment. For example, treatment may stabilize, maintain, improve or increase walking ability (e.g., stabilization of ambulation) in the human patient. In some embodiments, treatment maintains or increases a stable walking distance in a human patient, as measured by, for example, the 6 Minute Walk Test (6MWT), described by McDonald, et al. (Muscle Nerve, 2010; 42:966-74, herein incorporated by reference). A change in the 6 Minute Walk Distance (6MWD) may be expressed as an absolute value, a percentage change or a change in the %-predicted value.

Loss of muscle function in patients with DMD may occur against the background of normal childhood growth and development. Indeed, younger children with DMD may show an increase in distance walked during 6MWT over the course of about 1 year despite progressive muscular impairment. In some embodiments, the 6MWD from patients with DMD is compared to typically developing control subjects and to existing normative data from age and sex matched subjects (i.e., patients). In some embodiments, normal growth and development can be accounted for using an age and height based equation fitted to normative data. Such an equation can be used to convert 6MWD to a percent-predicted (%-predicted) value in patients with DMD. In certain embodiments, analysis of %-predicted 6MWD data represents a method to account for normal growth and development, and may show that gains in function at early ages (e.g., less than or equal to age 7) represent stable rather than improving abilities in patients with DMD (Henricson et al. PLoS Curr., 2012, version 2, herein incorporated by reference).

A “pediatric patient” as used herein is a human patient from age 1 to 21, inclusive. In one embodiment, the pediatric patient is a human patient from age 7 to 21, inclusive.

“Alkyl” or “alkylene” both refer to a saturated straight or branched hydrocarbon. In certain embodiments, the alkyl group is a primary, secondary, or tertiary hydrocarbon. In certain embodiments, the alkyl group includes one to ten carbon atoms, i.e., C₁ to C₁₀ alkyl. In certain embodiments, the alkyl group includes one to six carbon atoms, i.e., C₁ to C₆ alkyl. The term includes both substituted and unsubstituted alkyl groups, including halogenated alkyl groups. In certain embodiments, the alkyl group is a fluorinated alkyl group. Non-limiting examples of moieties with which the alkyl group can be substituted are selected from the group consisting of halogen (fluoro, chloro, bromo, or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. In certain embodiments, the alkyl group is selected from the group consisting of methyl, CF₃, CCl₃, CFCl₂, CF₂Cl, ethyl, CH₂CF₃, CF₂CF₃, propyl, isopropyl, butyl, isobutyl, sec-butyl, t-butyl, pentyl, isopentyl, neopentyl, hexyl, isohexyl, 3-methylpentyl, 2,2-dimethylbutyl, and 2,3-dimethylbutyl.

“Alkenyl” refers to an unsaturated straight or branched chain hydrocarbon radical containing from 2 to 18 carbons and comprising at least one carbon to carbon double bond. Examples include without limitation ethenyl, propenyl, iso-propenyl, butenyl, iso-butenyl, tert-butenyl, n-pentenyl and n-hexenyl. The term “lower alkenyl” refers to an alkenyl group, as defined herein, containing between 2 and 8 carbons.

“Alkynyl” refers to an unsaturated straight or branched chain hydrocarbon radical containing from 2 to 18 carbons comprising at least one carbon to carbon triple bond. Examples include without limitation ethynyl, propynyl, iso-propynyl, butynyl, iso-butynyl, tert-butynyl, pentynyl and hexynyl. The term “lower alkynyl” refers to an alkynyl group, as defined herein, containing between 2 and 8 carbons.

“Cycloalkyl” refers to a mono- or poly-cyclic alkyl radical. Examples include without limitation cyclobutyl, cyclopentyl, cyclohexyl, cycloheptyl and cyclooctyl.

The term “aryl” used alone or as part of a larger moiety as in “aralkyl,” “aralkoxy,” or “aryloxy-alkyl,” refers to aromatic ring groups having six to fourteen ring atoms, such as phenyl, 1-naphthyl, 2-naphthyl, 1-anthracyl and 2-anthracyl. An “aryl” ring may contain one or more substituents. The term “aryl” may be used interchangeably with the term “aryl ring.” “Aryl” also includes fused polycyclic aromatic ring systems in which an aromatic ring is fused to one or more rings. Non-limiting examples of useful aryl ring groups include phenyl, hydroxyphenyl, halophenyl, alkoxyphenyl, dialkoxyphenyl, trialkoxyphenyl, alkylenedioxyphenyl, naphthyl, phenanthryl, anthryl, phenanthro and the like, as well as 1-naphthyl, 2-naphthyl, 1-anthracyl and 2-anthracyl. Also included within the scope of the term “aryl,” as it is used herein, is a group in which an aromatic ring is fused to one or more non-aromatic rings, such as in an indanyl, phenanthridinyl, or tetrahydronaphthyl, where the radical or point of attachment is on the aromatic ring.

The term “acyl” refers to a C(O)R¹¹ group (in which R¹¹ signifies H, alkyl or aryl as defined herein). Examples of acyl groups include formyl, acetyl, benzoyl, phenylacetyl and similar groups.

“Aralkyl” refers to a radical of the formula —R¹²R¹³ where R¹² is an alkylene chain as defined above and R¹³ is one or more aryl radicals as defined above, for example, benzyl, diphenylmethyl and the like.

“Thioalkoxy” refers to a radical of the formula —SR¹⁴ where R¹⁴ is an alkyl radical as defined herein. The term “lower thioalkoxy” refers to an alkoxy group, as defined herein, containing between 1 and 8 carbons.

“Alkoxy” refers to a radical of the formula —OR¹⁵ where R¹⁵ is an alkyl radical as defined herein. The term “lower alkoxy” refers to an alkoxy group, as defined herein, containing between 1 and 8 carbons. Examples of alkoxy groups include, without limitation, methoxy and ethoxy.

“Alkoxyalkyl” refers to an alkyl group substituted with an alkoxy group.

“Carbonyl” refers to the C(═O)— radical.

“Guanidynyl” refers to the H₂N(C═NH₂)—NH— radical.

“Amidinyl” refers to the H₂N(C═NH₂)CH— radical.

“Amino” refers to the NH₂ radical.

“Alkylamino” refers to a radical of the formula —NHR¹⁶ or —NR¹⁶R¹⁶ where each R¹⁶ is, independently, an alkyl radical as defined herein. The term “lower alkylamino” refers to an alkylamino group, as defined herein, containing between 1 and 8 carbons.

“Heterocycle” means a 5- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined below. Thus, in addition to the heteroaryls listed below, heterocycles also include morpholinyl, pyrrolidinonyl, pyrrolidinyl, piperidinyl, piperizinyl, hydantoinyl, valerolactamyl, oxiranyl, oxetanyl, tetrahydrofuranyl, tetrahydropyranyl, tetrahydropyridinyl, tetrahydrothiophenyl, tetrahydrothiopyranyl, tetrahydropyrimidinyl, tetrahydrothiopyranyl, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, and quinazolinyl.

The terms “optionally substituted alkyl”, “optionally substituted alkenyl”, “optionally substituted alkoxy”, “optionally substituted thioalkoxy”, “optionally substituted alkyl amino”, “optionally substituted lower alkyl”, “optionally substituted lower alkenyl”, “optionally substituted lower alkoxy”, “optionally substituted lower thioalkoxy”, “optionally substituted lower alkyl amino” and “optionally substituted heterocyclyl” mean that, when substituted, at least one hydrogen atom is replaced with a substituent. In the case of an oxo substituent (═O) two hydrogen atoms are replaced. In this regard, substituents include: deuterium, optionally substituted alkyl, optionally substituted alkenyl, optionally substituted alkynyl, optionally substituted aryl, optionally substituted heterocycle, optionally substituted cycloalkyl, oxo, halogen, —CN, —OR^{x}, NR^{x}R^{y}, NR^{x}C(═O)R^{y}, NR^{x}SO₂R^{y}, —NR^{x}C(═O)NR^{x}R^{y}, C(═O)R^{x}, C(═O)OR^{x}, C(═O)NR^{x}R^{y}, —SOₘR^{x} and —SOₘNR^{x}R^{y}, wherein m is 0, 1 or 2, R^{x} and R^{y} are the same or different and independently hydrogen, optionally substituted alkyl, optionally substituted alkenyl, optionally substituted alkynyl, optionally substituted aryl, optionally substituted heterocycle or optionally substituted cycloalkyl and each of said optionally substituted alkyl, optionally substituted alkenyl, optionally substituted alkynyl, optionally substituted aryl, optionally substituted heterocycle and optionally substituted cycloalkyl substituents may be further substituted with one or more of oxo, halogen, and —CN.

The phrases “systemic administration,” “administered systemically,” “peripheral administration” and “administered peripherally” as used herein mean the administration of a compound, drug or other material other than directly into the central nervous system, such that it enters the human patient's system and, thus, is subject to metabolism and other like processes, for example, subcutaneous administration.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. The phrase “pharmaceutically acceptable” means the substance or composition must be compatible, chemically and/or toxicologically, with the other ingredients comprising a formulation, and/or the human patient being treated therewith.

The phrase “pharmaceutically-acceptable carrier” as used herein means a non-toxic, inert solid, semi-solid or liquid filler, diluent, encapsulating material, or formulation auxiliary of any type. Some examples of materials which can serve as pharmaceutically acceptable carriers are: sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening agents; flavoring agents; perfuming agents; preservatives; and antioxidants; according to the judgment of the formulator.

As used herein, a set of brackets used within a structural formula indicate that the structural feature between the brackets is repeated. In some embodiments, the brackets used can be “[” and “],” and in certain embodiments, brackets used to indicate repeating structural features can be “(” and “).” In some embodiments, the number of repeat iterations of the structural feature between the brackets is the number indicated outside the brackets such as 2, 3, 4, 5, 6, 7, and so forth. In various embodiments, the number of repeat iterations of the structural feature between the brackets is indicated by a variable indicated outside the brackets such as “Z”.

As used herein, a straight bond or a squiggly bond drawn to a chiral carbon or phosphorous atom within a structural formula indicates that the stereochemistry of the chiral carbon or phosphorous is undefined and is intended to include all forms of the chiral center and/or mixtures thereof. Examples of such illustrations are depicted below.

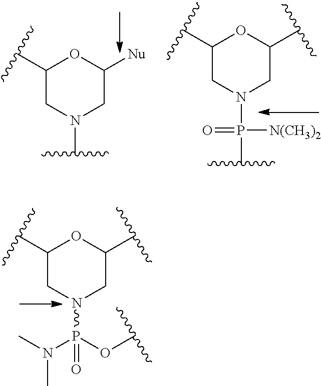

For clarity, structures of the disclosure including, for example, the above PPMO #1, PPMO #2, and PPMO #3 are continuous from 5′ to 3′, and, for the convenience of depicting the entire structure in a compact form, various illustration breaks labeled “BREAK A,” “BREAK B,” and “BREAK C” have been included. As would be understood by the skilled artisan, for example, each indication of “BREAK A” shows a continuation of the illustration of the structure at these points. The skilled artisan understands that the same is true for each instance of “BREAK B” and for “BREAK C” in the structures above. None of the illustration breaks, however, are intended to indicate, nor would the skilled artisan understand them to mean, an actual discontinuation of the structure above.

An antisense molecule nomenclature system was proposed and published to distinguish between the different antisense molecules. This nomenclature became especially relevant when testing several slightly different antisense molecules, all directed at the same target region, as shown below:

H#A/D(x:y).

The first letter designates the species (e.g. H: human, M: murine, C: canine). “#” designates target dystrophin exon number. “A/D” indicates acceptor or donor splice site at the beginning and end of the exon, respectively. (x y) represents the annealing coordinates where “−” or “+” indicate intronic or exonic sequences respectively. For example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2-18) where the last 2 exonic bases and the first 18 intronic bases correspond to the annealing site of the antisense molecule. Entirely exonic annealing coordinates that would be represented by A(+65+85), that is the site between the 65th and 85th nucleotide from the start of that exon.

### II. METHODS OF THE DISCLOSURE

The present disclosure is directed to methods of treating a human patient having a disease or disorder amenable to antisense oligonucleotide therapy by administering an effective amount of an antisense oligonucleotide (e.g., PMO) or antisense oligonucleotide conjugate (e.g., PPMO), or a pharmaceutically acceptable salt thereof. The effective amount of the antisense oligonucleotide or antisense oligonucleotide conjugate, or pharmaceutically acceptable salt thereof, can be administered every one, two, three, or four weeks. In some embodiments, the antisense oligonucleotide (e.g., PMO) or antisense oligonucleotide conjugate (e.g., PPMO), or pharmaceutically acceptable salt thereof, is administered once every four weeks. In some embodiments, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #1 dissolved in an aqueous carrier solution is administered every four weeks. In some embodiments, 20 mg/kg, 30 mg/kg, 40 mg/kg, 50 mg/kg, 60 mg/kg, 70 mg/kg, 80 mg/kg, 90 mg/kg, or 100 mg/kg of PPMO #16HCl dissolved in an aqueous carrier solution is administered every four weeks. In some embodiments, about 60 mg/kg of a pharmaceutically acceptable salt of PPMO #1 dissolved in an aqueous carrier solution is administered every four weeks. In some embodiments, about 80 mg/kg of a pharmaceutically acceptable salt of PPMO #1 dissolved in an aqueous carrier solution is administered every four weeks. In some embodiments, about 100 mg/kg of a pharmaceutically acceptable salt of PPMO #1 dissolved in an aqueous carrier solution is administered every four weeks. In some embodiments, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #2 dissolved in an aqueous carrier solution is administered every four weeks. In some embodiments, the about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #3 dissolved in an aqueous carrier solution is administered every four weeks. In one embodiment, the effective amount of the antisense oligonucleotide or antisense oligonucleotide conjugate, or pharmaceutically acceptable salt thereof, is administered monthly.

In one embodiment, the methods are directed to treating a human patient having muscular dystrophy (e.g., DMD) comprising administering an antisense oligonucleotide (e.g., PMO) or an antisense oligonucleotide conjugate (e.g., PPMO), or a pharmaceutically acceptable salt thereof, once every four weeks. In some embodiments, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #1 dissolved in an aqueous carrier solution is administered every four weeks. In some embodiments, 20 mg/kg, 30 mg/kg, 40 mg/kg, 50 mg/kg, 60 mg/kg, 70 mg/kg, 80 mg/kg, 90 mg/kg, or 100 mg/kg of PPMO #1.6HCl dissolved in an aqueous carrier solution is administered every four weeks. In some embodiments, about 60 mg/kg of a pharmaceutically acceptable salt of PPMO #1 dissolved in an aqueous carrier solution is administered every four weeks. In some embodiments, about 80 mg/kg of a pharmaceutically acceptable salt of PPMO #1 dissolved in an aqueous carrier solution is administered every four weeks. In some embodiments, about 100 mg/kg of a pharmaceutically acceptable salt of PPMO #1 dissolved in an aqueous carrier solution is administered every four weeks. In some embodiments, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #2 dissolved in an aqueous carrier solution is administered every four weeks. In some embodiments, the about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg of a pharmaceutically acceptable salt of PPMO #3 dissolved in an aqueous carrier solution is administered every four weeks. In one embodiment, the effective amount of the antisense oligonucleotide or antisense oligonucleotide conjugate, or pharmaceutically acceptable salt thereof, is administered monthly.

Some aspects of the present disclosure are directed to methods of increasing or restoring a muscle cell condition in a human patient having a muscular dystrophy (e.g., DMD) comprising once every four weeks administering to the human patient an effective amount of an antisense oligonucleotide (e.g., PMO) or an antisense oligonucleotide conjugate (e.g., PPMO), or a pharmaceutically acceptable salt thereof, that is complementary to a nucleotide sequence within a dystrophin transcript and is capable of inducing exon skipping in the dystrophin transcript. In one embodiment, the effective amount of the antisense oligonucleotide or antisense oligonucleotide conjugate, or pharmaceutically acceptable salt thereof, is administered monthly.

### Antisense Oligonucleotides Designed to Induce Exon Skipping

In certain embodiments, the antisense oligonucleotide or antisense oligonucleotide conjugate induces skipping of one or more exons or a portion thereof in the transcript. In certain embodiments, the one or more exons or a portion thereof are selected from group consisting of exon 44, exon 45, exon 50, exon 51, exon 52, exon 53, and any combination thereof. In certain embodiments, the antisense oligonucleotide or antisense oligonucleotide conjugate induces skipping of exon 51, exon 45, or exon 53 of the dystrophin transcript.

In certain embodiments, the antisense oligonucleotide or antisense oligonucleotide conjugate induces skipping of exon 51 of the dystrophin transcript. In certain embodiments, the antisense oligonucleotide or antisense oligonucleotide conjugate comprises a base sequence that is complementary to an exon 51 target region of the dystrophin transcript designated as an annealing site, wherein the base sequence and annealing site are selected from:

| Annealing Site | Base Sequence [5′ to 3′] | SEQ ID NO. |
|---|---|---|
| H51A(+66+95) | CTC CAA CAT CAA GGA AGA TGG CAT TTC TAG | 1 |
| H51A(+74+97) | ACC TCC AAC ATC AAG GAA GAT GGC | 2 |
| H51A(+70+99) | GTA CCT CCA ACA TCA AGG AAG ATG GCA TTT | 3 |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |

wherein each T of each of SEQ ID NOS: 1-6 is thymine or uracil. In certain embodiments, the T in the antisense oligonucleotide or antisense oligonucleotide conjugate is thymine. In certain embodiments, the T in the antisense oligonucleotide or antisense oligonucleotide conjugate is uracil.

In certain embodiments, the antisense oligonucleotide or antisense oligonucleotide conjugate induces skipping of exon 53 of the dystrophin transcript. In certain embodiments, the antisense oligonucleotide or antisense oligonucleotide conjugate comprises a base sequence that is complementary to an exon 53 target region of the dystrophin transcript designated as an annealing site, wherein the base sequence and annealing site are selected from:

| Annealing Site | Base Sequence [5′ to 3′] | SEQ ID NO. |
|---|---|---|
| H53A(+36+60) | GTT GCC TCC GGT TCT GAA GGT GTT C | 7 |
| H53A(+36+60) | GTT G5mC5mC T5mC5mC GGT T5mC T GAA GGT GTT 5mC | 8 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG TAC | 13 |
| H53A(+39+62) | CTG TTG CCT CCG GTT CTG AAG GTG | 14 |
| H53A(+36+69) | CAT TCA ACT GTT GCC TCC GGT TCT GAA GGT G | 15 |
| H53A(+45+62) | CTG TTG CCT CCG GTT CTG | 16 |

wherein each T of each of SEQ ID NOS: 7-16 is thymine or uracil. In certain embodiments, the T in the antisense oligonucleotide or antisense oligonucleotide conjugate is thymine. In certain embodiments, the T in the antisense oligonucleotide or antisense oligonucleotide conjugate is uracil.

In certain embodiments, the antisense oligonucleotide or antisense oligonucleotide conjugate induces skipping of exon 45 of the dystrophin transcript. In certain embodiments, the antisense oligonucleotide or antisense oligonucleotide conjugate comprises a base sequence that is complementary to an exon 45 target region of the dystrophin transcript designated as an annealing site, wherein the base sequence and annealing site are selected from:

| Annealing Site | Base Sequence [5′ to 3′] | SEQ ID NO. |
|---|---|---|
| H45A(-03+19) | CAA TGC CAT CCT GGA GTT CCT G | 17 |
| H45A(-09+25) | GCT GCC CAA TGC CAT CCT GGA OTT CCT GTA AGA T | 18 |
| H45A(-03+25) | GCT GCC CAA TGC CAT CCT GGA GTT CCT G | 19 |
| H45A(-06+25) | GCT GCC CAA TGC CAT CCT GGA GTT CCT GTA A | 20 |
| H45A(-12+19) | CAA TGC CAT CCT GGA GTT CCT GTA AGA TAC C | 21 |
| H45A(-09+19) | CAA TGC CAT CCT GGA GTT CCT GTA AGA T | 22 |
| H45A(-12+16) | TGC CAT CCT GGA GTT CCT GTA AGA TAC C | 23 |
| H45A(-14+25) | GCT GCC CAA TGC CAT CCT GGA GTT CCT GTA AGA | 24 |
| | TAC CAA | |
| H45A(-08+19) | CAA TGC CAT CCT GGA GTT CCT GTA AGA | 25 |
| HM45A | GCT GCC CAA TGC CAT CCT GGA GTT CCT GTA AG | 26 |
| (-07+25) | | |
| H45A(-12+22) | GCC CAA TGC CAT CCT GGA GTT CCT GTA AGA TAC C | 27 |
| H45A(-09+22) | GCC CAA TGC CAT CCT GGA GTT CCT GTA AGA T | 28 |
| H45A(-09+30) | TTG CCG CTG CCC AAT GCC ATC CTG GAG TTC CTG | 29 |
| | TAA GAT | |
| H45A(-06+22) | GCC CAA TGC CAT CCT GGA GTT CCT GTA A | 30 |
| H45A(-06+28) | GCC GCT GCC CAA TGC CAT CCT GGA GTT CCT GTA A | 31 |
| H45A(-03+22) | GCC CAA TGC CAT CCT GGA GTT CCT G | 32 |
| H45A(-03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A-U-m5C- | 34 |
| | m5C | |

wherein each T of each of SEQ ID NOS: 17-34 is thymine or uracil. In certain embodiments, the T in the antisense oligonucleotide or antisense oligonucleotide conjugate is thymine. In certain embodiments, the T in the antisense oligonucleotide or antisense oligonucleotide conjugate is uracil.

In certain embodiments, the antisense oligonucleotide or antisense oligonucleotide conjugate induces skipping of exon 44 of the dystrophin transcript. In certain embodiments, the antisense oligonucleotide or antisense oligonucleotide conjugate comprises a base sequence that is complementary to an exon 44 target region of the dystrophin transcript designated as an annealing site, wherein the base sequence and annealing site are selected from:

| Annealing Site | Base Sequence [5′ to 3′] | SEQ ID NO: |
|---|---|---|
| H44A(-10+15) | GAT CTG TCA AAT CGC CTG CAG GTA A | 35 |
| H44A(-07+15) | GAT CTG TCA AAT CGC CTG CAG G | 36 |
| H44M(-07+17) | CAG ATC TGT CAA ATC GCC TGC AGG | 37 |
| H44A(-08+15) | GAT CTG TCA AAT CGC CTG CAG GT | 38 |
| H44A(-06+15) | GAT CTG TCA AAT CGC CTG CAG | 39 |
| H44A(-08+17) | CAG ATC TGT CAA ATC GCC TGC AGG T | 40 |
| H44A(-06+17) | CAG ATC TGT CAA ATC GCC TGC AG | 41 |

wherein each T of each of SEQ ID NOS: 35-41 is thymine or uracil. In certain embodiments, the T in the antisense oligonucleotide or antisense oligonucleotide conjugate is thymine. In certain embodiments, the T in the antisense oligonucleotide or antisense oligonucleotide conjugate is uracil.

In certain embodiments, the antisense oligonucleotide or antisense oligonucleotide conjugate induces skipping of exon 50 of the dystrophin transcript. In certain embodiments, the antisense oligonucleotide or antisense oligonucleotide conjugate comprises a base sequence that is complementary to an exon 50 target region of the dystrophin transcript designated as an annealing site, wherein the base sequence and annealing site are selected from:

| Annealing Site | Targeting Sequence [5′ to 3′] | SEQ ID NO: |
|---|---|---|
| H50D(+04-18) | GGG ATC CAG TAT ACT TAC AGG C | 42 |
| H50D(+07-18) | GGG ATC CAG TAT ACT TAC AGG CTC C | 43 |
| H50D(+07-16) | GAT CCA GTA TAC TTA CAG GCT CC | 44 |
| H50D(+07-17) | GGA TCC AGT ATA CTT ACA GGC TCC | 45 |
| H50A(-19+07) | ACT TCC TCT TTA ACA GAA AAG CAT AC | 46 |
| H50D(+07-15) | ATC CAG TAT ACT TAC AGG CTC C | 47 |
| H50A(-02+23) | GAG CTC AGA TCT TCT AAC TTC CTC T | 48 |
| H50D(+06-18) | GGG ATC CAG TAT ACT TAC AGG CTC | 49 |
| H50D(+07-20) | ATG GGA TCC AGT ATA CTT ACA GGC TCC | 50 |

wherein each T of each of SEQ ID NOS: 42-50 is thymine or uracil. In certain embodiments, the T in the antisense oligonucleotide or antisense oligonucleotide conjugate is thymine. In certain embodiments, the T in the antisense oligonucleotide or antisense oligonucleotide conjugate is uracil.

In certain embodiments, the antisense oligonucleotide or antisense oligonucleotide conjugate induces skipping of exon 52 of the dystrophin transcript. In certain embodiments, the antisense oligonucleotide or antisense oligonucleotide conjugate comprises a base sequence that is complementary to an exon 52 target region of the dystrophin transcript designated as an annealing site, wherein the base sequence and annealing site are selected from:

| Annealing Site | Targeting Sequence [5′ to 3′] | SEQ ID NO: |
|---|---|---|
| H52A(-01+24) | CTG TTC CAA ATC CTG CAT TGT TGC C | 51 |

wherein each T of SEQ ID NO: 51 is thymine or uracil. In certain embodiments, the T in the antisense oligonucleotide or antisense oligonucleotide conjugate is thymine. In certain embodiments, the T in the antisense oligonucleotide or antisense oligonucleotide conjugate is uracil.

In some embodiments, an antisense oligonucleotide or antisense oligonucleotide conjugate is a PMO or PPMO wherein each morpholino ring of the PMO or PPMO is linked to a nucleobase including, for example, nucleobases found in DNA (adenine, cytosine, guanine, and thymine).

The antisense oligomers or antisense oligomer conjugates of the disclosed methods can employ a variety of antisense oligomer chemistries. Examples of oligomer chemistries include, without limitation, morpholino oligomers, phosphorothioate modified oligomers, 2′ O-methyl modified oligomers, peptide nucleic acid (PNA), locked nucleic acid (LNA), phosphorothioate oligomers, 2′ O-MOE modified oligomers, 2′-fluoro-modified oligomer, 2′O,4′C-ethylene-bridged nucleic acids (ENAs), tricyclo-DNAs, tricyclo-DNA phosphorothioate subunits, 2′-O-[2-(N-methylcarbamoyl)ethyl]modified oligomers, including combinations of any of the foregoing. Phosphorothioate and 2′-O-Me-modified chemistries can be combined to generate a 2′O-Me-phosphorothioate backbone. See, e.g., PCT Publication Nos. WO/2013/112053 and WO/2009/008725, which are hereby incorporated by reference in their entireties. Exemplary embodiments of oligomer chemistries of the disclosure are further described herein.

### A. Peptide Nucleic Acids (PNAs)

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligomers obeying Watson-Crick base-pairing rules, and mimic DNA in terms of base pair recognition. The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases. A non-limiting example of a PNA is depicted below.

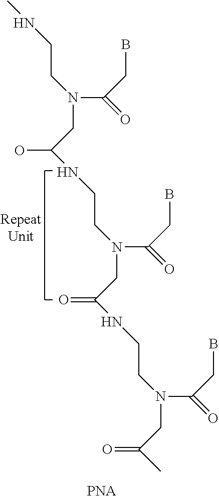

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766; 7,211,668; 7,022,851; 7,125,994; 7,145,006; and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

### B. Locked Nucleic Acids (LNAs)

Antisense oligomers may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4-C positions. LNA enhances backbone preorganization and base stacking to increase hybridization and thermal stability.

The structures of LNAs can be found, for example, in Wengel, et al., Chemical Communications (1998) 455; Koshkin et al., Tetrahedron (1998) 54:3607; Jesper Wengel, Accounts of Chem. Research (1999) 32:301; Obika, et al., Tetrahedron Letters (1997) 38:8735; Obika, et al., Tetrahedron Letters (1998) 39:5401; and Obika, et al., Bioorganic Medicinal Chemistry (2008) 16:9230, which are hereby incorporated by reference in their entirety. A non-limiting example of an LNA is depicted below.

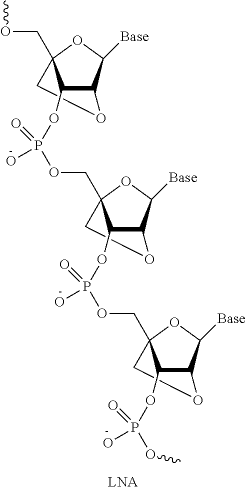

Antisense oligomers of the disclosure may incorporate one or more LNAs; in some cases, the antisense oligomers may be entirely composed of LNAs. Methods for the synthesis of individual LNA nucleoside subunits and their incorporation into oligomers are described, for example, in U.S. Pat. Nos. 7,572,582; 7,569,575; 7,084,125; 7,060,809; 7,053,207; 7,034,133; 6,794,499; and 6,670,461; each of which is incorporated by reference in its entirety. Typical intersubunit linkers include phosphodiester and phosphorothioate moieties; alternatively, non-phosphorous containing linkers may be employed. Further embodiments include an LNA containing antisense oligomer where each LNA subunit is separated by a DNA subunit. Certain antisense oligomers are composed of alternating LNA and DNA subunits where the intersubunit linker is phosphorothioate.

2′O,4′C-ethylene-bridged nucleic acids (ENAs) are another member of the class of BNAs. A non-limiting example is depicted below.

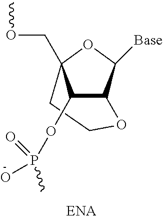

ENA oligomers and their preparation are described in Obika et al., Tetrahedron Lett (1997) 38 (50): 8735, which is hereby incorporated by reference in its entirety, Antisense oligomers of the disclosure may incorporate one or more ENA subunits.

### C. Unlocked Nucleic Acid (UNA)

Antisense oligomers may also contain unlocked nucleic acid (UNA) subunits. UNAs and UNA oligomers are an analogue of RNA in which the C2′-C3′ bond of the subunit has been cleaved. Whereas LNA is conformationally restricted (relative to DNA and RNA), UNA is very flexible. UNAs are disclosed, for example, in WO 2016/070166. A non-limiting example of an UNA is depicted below.

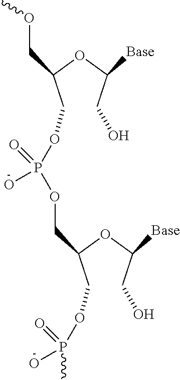

Typical intersubunit linkers include phosphodiester and phosphorothioate moieties; alternatively, non-phosphorous containing linkers may be employed.

### D. Phosphorothioates

“Phosphorothioates” (or S-oligos) are a variant of normal DNA in which one of the nonbridging oxygens is replaced by a sulfur. A non-limiting example of a phosphorothioate is depicted below.

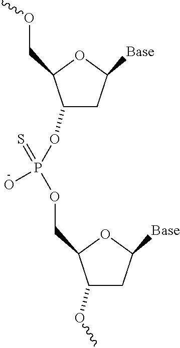

The sulfurization of the internucleotide bond reduces the action of endo- and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990, which is hereby incorporated by reference in its entirety). The latter methods avoid the problem of elemental sulfur's insolubility in most organic solvents and the toxicity of carbon disulfide. The TETD and BDTD methods also yield higher purity phosphorothioates.

### E. Triccyclo-DNAs and Tricyclo-Phosphorothioate Subunits

Tricyclo-DNAs (tc-DNA) are a class of constrained DNA analogs in which each nucleotide is modified by the introduction of a cyclopropane ring to restrict conformational flexibility of the backbone and to optimize the backbone geometry of the torsion angle γ. Homobasic adenine- and thymine-containing tc-DNAs form extraordinarily stable A-T base pairs with complementary RNAs. Tricyclo-DNAs and their synthesis are described in International Patent Application Publication No. WO 2010/115993, which is hereby incorporated by reference in its entirety. Antisense oligomers of the disclosure may incorporate one or more tricycle-DNA subunits; in some cases, the antisense oligomers may be entirely composed of tricycle-DNA subunits.

Tricyclo-phosphorothioate subunits are tricyclo-DNA subunits with phosphorothioate intersubunit linkages. Tricyclo-phosphorothioate subunits and their synthesis are described in International Patent Application Publication No. WO 2013/053928, which is hereby incorporated by reference in its entirety. Antisense oligomers of the disclosure may incorporate one or more tricycle-DNA subunits; in some cases, the antisense oligomers may be entirely composed of tricycle-DNA subunits. A non-limiting example of a tricycle-DNA/tricycle-phophothioate subunit is depicted below.

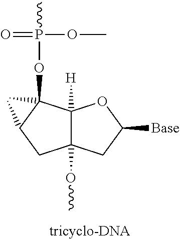

### F. 2′ O-Methyl, 2′ O-MOE, and 2′-F Oligomers

“2′-O-Me oligomer” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphorothioate oligomers (PTOs) for further stabilization. 2′O-Me oligomers (phosphodiester or phosphothioate) can be synthesized according to routine techniques in the art (see, e.g., Yoo et al., Nucleic Acids Res. 32:2008-16, 2004, which is hereby incorporated by reference in its entirety). A non-limiting example of a 2′ O-Me oligomer is depicted below.

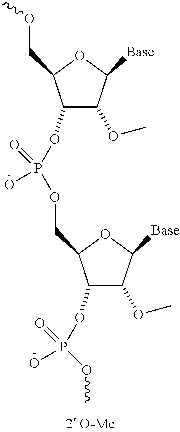

2′ O-Methoxyethyl Oligomers (2′-O MOE) carry a methoxyethyl group at the 2′-OH residue of the ribose molecule and are discussed in Martin et al., Helv. Chim. Acta, 78, 486-504, 1995, which is hereby incorporated by reference in its entirety A non-limiting example of a 2′O MOE subunit is depicted below.

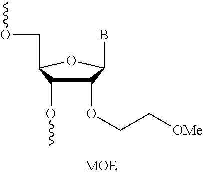

2′-Fluoro (2′-F) oligomers have a fluoro radical in at the 2′ position in place of the 2′OH. A non-limiting example of a 2′-F oligomer is depicted below.

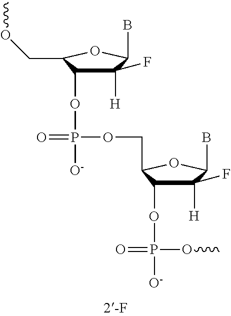

2′-Fluoro oligomers are further described in WO 2004/043977, which is hereby incorporated by reference in its entirety.

2′O-Methyl, 2′ O-MOE, and 2′-F oligomers may also comprise one or more phosphorothioate (PS) linkages as depicted below.

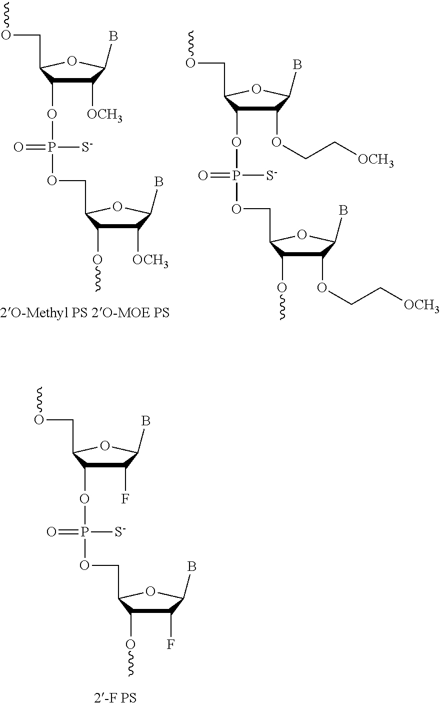

Additionally, 2′O-Methyl, 2′ O-MOE, and 2′-F oligomers may comprise PS intersubunit linkages throughout the oligomer, for example, as in the 2′O-methyl PS oligomer drisapersen depicted below.

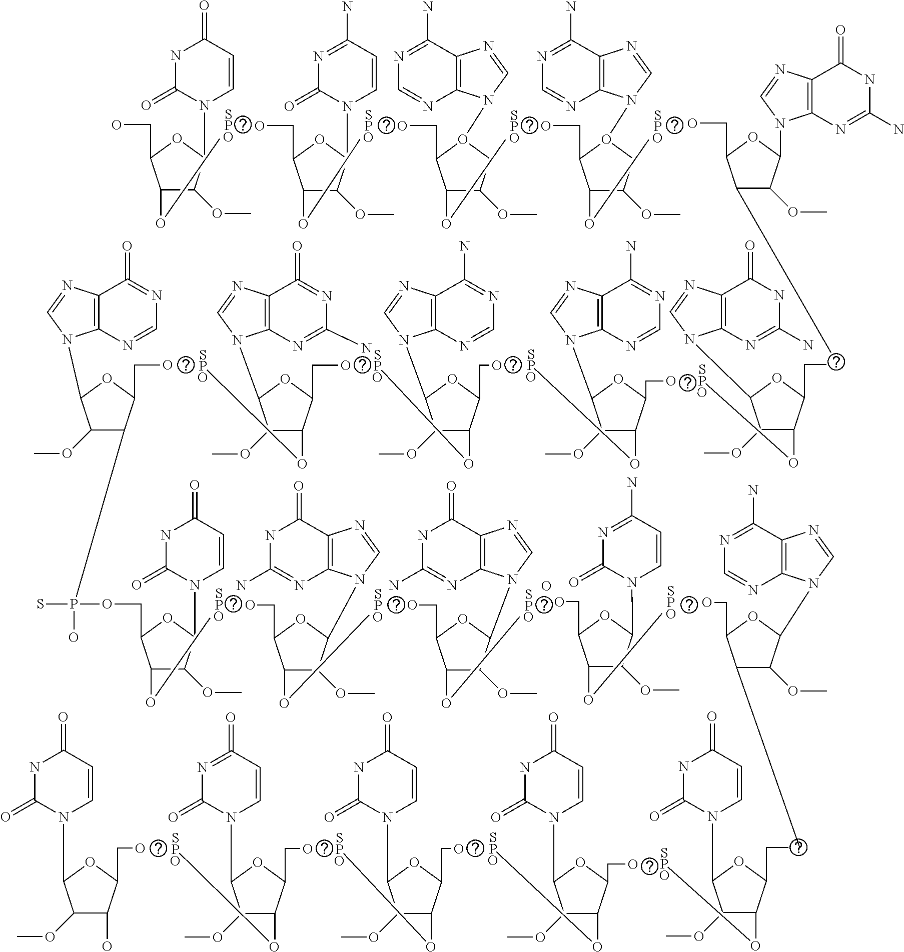

Alternatively, 2′O-Methyl, 2′ O-MOE, and/or 2′-F oligomers may comprise PS linkages at the ends of the oligomer, as depicted below.

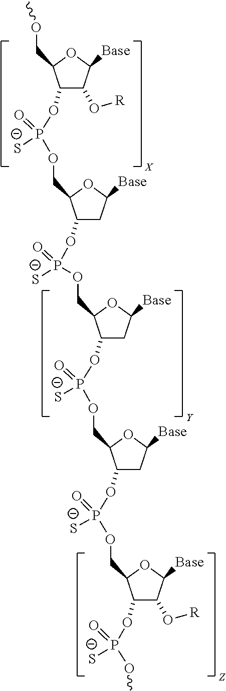

where:
R is CH₂CH₂OCH₃ (methoxyethyl or MOE); and
x, y, and z denote the number of nucleotides contained within each of the designated 5′-wing, central gap, and 3-wing regions, respectively.

Antisense oligomers of the disclosure may incorporate one or more 2′ O-Methyl, 2′ O-MOE, and 2′-F subunits and may utilize any of the intersubunit linkages described here. In some instances, an antisense oligomer of the disclosure may be composed of entirely 2′O-Methyl, 2′ O-MOE, or 2′-F subunits. One embodiment of an antisense oligomers of the disclosure is composed entirely of 2′O-methyl subunits.

### G. 2′-O-[2-(N-methylcarbamoyl)ethyl] Oligomers (MCEs)

MCEs are another example of 2′O modified ribonucleosides useful in the antisense oligomers of the disclosure. Here, the 2′OH is derivatized to a 2-(N-methylcarbamoyl)ethyl moiety to increase nuclease resistance. A non-limiting example of an MCE oligomer is depicted below.

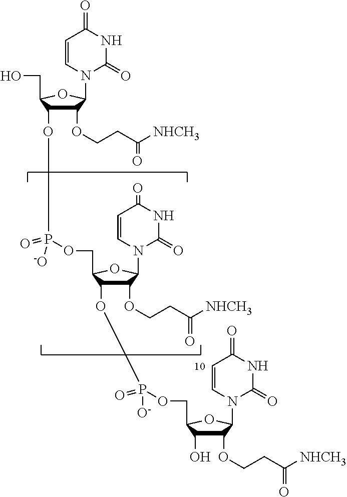

MCEs and their synthesis are described in Yamada et al., J. Org. Chem. (2011) 76(9):3042-53, which is hereby incorporated by reference in its entirety. Antisense oligomers of the disclosure may incorporate one or more MCE subunits.

### H. Stereo Specific Oligomers

Stereo specific oligomers are those in which the stereo chemistry of each phosphorous-containing linkage is fixed by the method of synthesis such that a substantially stereo-pure oligomer is produced. A non-limiting example of a stereo specific oligomer is depicted below.

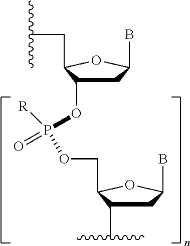

In the above example, each phosphorous of the oligomer has the same stereo configuration. Additional examples include the oligomers described above. For example, LNAs, ENAs, Tricyclo-DNAs, MCEs, 2′ O-Methyl, 2′ O-MOE, 2′-F, and morpholino-based oligomers can be prepared with stereo-specific phosphorous-containing internucleoside linkages such as, for example, phosphorothioate, phosphodiester, phosphoramidate, phosphorodiamidate, or other phosphorous-containing internucleoside linkages. Stereo specific oligomers, methods of preparation, chiral controlled synthesis, chiral design, and chiral auxiliaries for use in preparation of such oligomers are detailed, for example, in WO2017192664, WO2017192679, WO2017062862, WO2017015575, WO2017015555, WO2015107425, WO2015108048, WO2015108046, WO2015108047, WO2012039448, WO2010064146, WO2011034072, WO2014010250, WO2014012081, WO20130127858, and WO2011005761, each of which is hereby incorporated by reference in its entirety.

Stereo specific oligomers can have phosphorous-containing internucleoside linkages in an R_{P} or S_{P} configuration. Chiral phosphorous-containing linkages in which the stereo configuration of the linkages is controlled is referred to as “stereopure,” while chiral phosphorous-containing linkages in which the stereo configuration of the linkages is uncontrolled is referred to as “stereorandom.” In certain embodiments, the oligomers of the disclosure comprise a plurality of stereopure and stereorandom linkages, such that the resulting oligomer has stereopure subunits at pre-specified positions of the oligomer. An example of the location of the stereopure subunits is provided in international patent application publication number WO 2017/062862 A2 in FIGS. 7A and 7B. In an embodiment, all the chiral phosphorous-containing linkages in an oligomer are stereorandom. In an embodiment, all the chiral phosphorous-containing linkages in an oligomer are stereopure.

In an embodiment of an oligomer with n chiral phosphorous-containing linkages (where n is an integer of 1 or greater), all n of the chiral phosphorous-containing linkages in the oligomer are stereorandom. In an embodiment of an oligomer with n chiral phosphorous-containing linkages (where n is an integer of 1 or greater), all n of the chiral phosphorous-containing linkages in the oligomer are stereopure. In an embodiment of an oligomer with n chiral phosphorous-containing linkages (where n is an integer of 1 or greater), at least 10% (to the nearest integer) of the n phosphorous-containing linkages in the oligomer are stereopure. In an embodiment of an oligomer with n chiral phosphorous-containing linkages (where n is an integer of 1 or greater), at least 20% (to the nearest integer) of the n phosphorous-containing linkages in the oligomer are stereopure. In an embodiment of an oligomer with n chiral phosphorous-containing linkages (where n is an integer of 1 or greater), at least 30% (to the nearest integer) of the n phosphorous-containing linkages in the oligomer are stereopure. In an embodiment of an oligomer with n chiral phosphorous-containing linkages (where n is an integer of 1 or greater), at least 40% (to the nearest integer) of the n phosphorous-containing linkages in the oligomer are stereopure. In an embodiment of an oligomer with n chiral phosphorous-containing linkages (where n is an integer of 1 or greater), at least 50% (to the nearest integer) of the n phosphorous-containing linkages in the oligomer are stereopure. In an embodiment of an oligomer with n chiral phosphorous-containing linkages (where n is an integer of 1 or greater), at least 60% (to the nearest integer) of the n phosphorous-containing linkages in the oligomer are stereopure. In an embodiment of an oligomer with n chiral phosphorous-containing linkages (where n is an integer of 1 or greater), at least 70% (to the nearest integer) of the n phosphorous-containing linkages in the oligomer are stereopure. In an embodiment of an oligomer with n chiral phosphorous-containing linkages (where n is an integer of 1 or greater), at least 80% (to the nearest integer) of the n phosphorous-containing linkages in the oligomer are stereopure. In an embodiment of an oligomer with n chiral phosphorous-containing linkages (where n is an integer of 1 or greater), at least 90% (to the nearest integer) of the n phosphorous-containing linkages in the oligomer are stereopure.

In an embodiment of an oligomer with n chiral phosphorous-containing linkages (where n is an integer of 1 or greater), the oligomer contains at least 2 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 3 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 4 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 5 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 6 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 7 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 8 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 9 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 10 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 11 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 12 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 13 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 14 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 15 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 16 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 17 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 18 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 19 contiguous stereopure phosphorous-containing linkages of the same stereo orientation (i.e. either S_{P} or R_{P}). In an embodiment of an oligomer with n chiral phosphorous-containing linkages (where n is an integer of 1 or greater), the oligomer contains at least 20 contiguous stereopure phosphorous-containing linkages of the same stereo orientation (i.e. either S_{P} or R_{P}).

### I. Morpholino Oligomers

Exemplary embodiments of the disclosure relate to phosphorodiamidate morpholino oligomers or phosphorodiamidate morpholino oligomer conjugates of the following general structure:

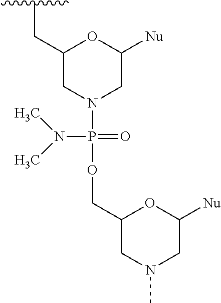

and as described in FIG. 2 of Summerton, J., et al., Antisense & Nucleic Acid Drug Development, 7: 187-195 (1997). Morpholinos as described herein are intended to cover all stereoisomers and tautomers of the foregoing general structure. The synthesis, structures, and binding characteristics of morpholino oligomers are detailed in U.S. Pat. Nos. 5,698,685; 5,217,866; 5,142,047; 5,034,506; 5,166,315; 5,521,063; 5,506,337; 8,076,476; and 8,299,206, all of which are incorporated herein by reference.

In certain embodiments, a morpholino is conjugated at the 5′ or 3′ end of the oligomer with a “tail” moiety to increase its stability and/or solubility. Exemplary tails include:

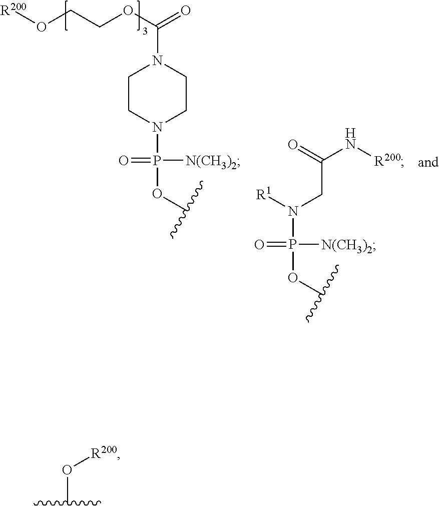

wherein R²⁰⁰ is is hydrogen or a cell-penetrating peptide and R¹ is C₁-C₆ alkyl. In one embodiment, R²⁰⁰ is hydrogen.

In various aspects, the antisense oligonucleotide or antisense oligonucleotide conjugate is according to Formula (I):

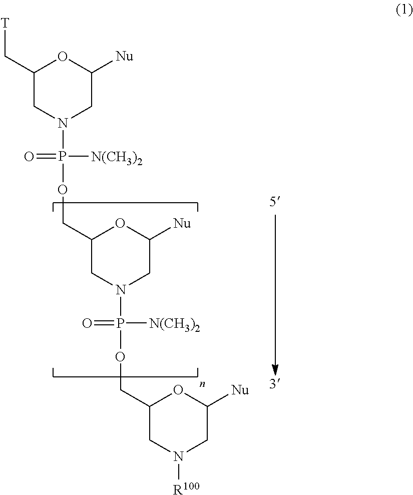

or a pharmaceutically acceptable salt thereof, wherein:
each Nu is a nucleobase which taken together form a targeting sequence;

T′ is a moiety selected from:

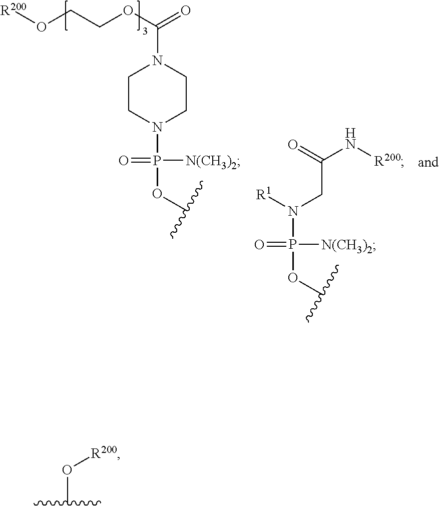

R¹⁰⁰ and R²⁰⁰ is independently hydrogen or a cell-penetrating peptide and R¹ is C₁-C₆ alkyl;
each Nu from 1 to (n+1) and 5′ to 3′ corresponds to the nucleobases in one of the following:

| Annealing Site | Targeting Sequence [5′ to 3′] | SEQ ID NO: |
|---|---|---|
| H51A(+66+95) | CTC CAA CAT CAA GGA AGA TGG CAT TTC TAG | 1 |
| H51A(+74+97) | ACC TCC AAC ATC AAG GAA GAT GGC | 2 |
| H51A(+70+99) | GTA CCT CCA ACA TCA AGG AAG ATG GCA TTT | 3 |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |
| H53A(+36+60) | GTT GCC TCC GGT TCT GAA GGT GTT C | 7 |
| H53A(+36+60) | GTT G5mC5mC T5mC5mC GGT T5mC T GAA GGT GTT 5mC | 8 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG TAC | 13 |
| H53A(+39+62) | CTG TTG CCT CCG GTT CTG AAG GTG | 14 |
| H53A(+36+69) | CAT TCA ACT GTT GCC TCC GGT TCT GAA GGT G | 15 |
| H53A(+45+62) | CTG TTG CCT CCG GTT CTG | 16 |
| H45A(-03+19) | CAA TGC CAT CCT GGA GTT CCT G | 17 |
| H45A(-09+25) | GCT GCC CAA TGC CAT CCT GGA GTT CCT GTA AGA T | 18 |
| H45A(-03+25) | GCT GCC CAA TGC CAT CCT GGA GTT CCT G | 19 |
| H45A(-06+25) | GCT GCC CAA TGC CAT CCT GGA GTT CCT GTA A | 20 |
| H45A(-12+19) | CAA TGC CAT CCT GGA GTT CCT GTA AGA TAC C | 21 |
| H45A(-09+19) | CAA TGC CAT CCT GGA GTT CCT GTA AGA T | 22 |
| H45A(-12+16) | TGC CAT CCT GGA GTT CCT GTA AGA TAC C | 23 |
| H45A(-14+25) | GCT GCC CAA TGC CAT CCT GGA GTT CCT GTA AGA | 24 |
| | TAC CAA | |
| H45A(-08+19) | CAA TGC CAT CCT GGA GTT CCT GTA AGA | 25 |
| HM45A(-07+25) | GCT GCC CAA TGC CAT CCT GGA GTT CCT GTA AG | 26 |
| H45A(-12+22) | GCC CAA TGC CAT CCT GGA GTT CCT GTA AGA TAC C | 27 |
| H45A(-09+22) | GCC CAA TGC CAT CCT GGA GTT CCT GTA AGA T | 28 |
| H45A(-09+30) | TTG CCG CTG CCC AAT GCC ATC CTG GAG TTC CTG TAA | 29 |
| | GAT | |
| H45A(-06+22) | GCC CAA TGC CAT CCT GGA GTT CCT GTA A | 30 |
| H45A(-06+28) | GCC GCT GCC CAA TGC CAT CCT GGA GTT CCT GTA A | 31 |
| H45A(-03+22) | GCC CAA TGC CAT CCT GGA GTT CCT G | 32 |
| H45A(-03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A-U-m5C- | 34 |
| | m5C | |
| H44A(-10+15) | GAT CTG TCA AAT CGC CTG CAG GTA A | 35 |
| H44A(-07+15) | GAT CTG TCA AAT CGC CTG CAG G | 36 |
| H44M(-07+17) | CAG ATC TGT CAA ATC GCC TGC AGG | 37 |
| H44A(-08+15) | GAT CTG TCA AAT CGC CTG CAG GT | 38 |
| H44A(-06+15) | GAT CTG TCA AAT CGC CTG CAG | 39 |
| H44A(-08+17) | CAG ATC TGT CAA ATC GCC TGC AGG T | 40 |
| H44A(-06+17) | CAG ATC TGT CAA ATC GCC TGC AG | 41 |
| H50D(+04-18) | GGG ATC CAG TAT ACT TAC AGG C | 42 |
| H50D(+07-18) | GGG ATC CAG TAT ACT TAC AGG CTC C | 43 |
| H50D(+07-16) | GAT CCA GTA TAC TTA CAG GCT CC | 44 |
| H50D(+07-17) | GGA TCC AGT ATA CTT ACA GGC TCC | 45 |
| H50A(-19+07) | ACT TCC TCT TTA ACA GAA AAG CAT AC | 46 |
| H50D(+07-15) | ATC CAG TAT ACT TAC AGG CTC C | 47 |
| H50A(-02+23) | GAG CTC AGA TCT TCT AAC TTC CTC T | 48 |
| H50D(+06-18) | GGG ATC CAG TAT ACT TAC AGG CTC | 49 |
| H50D(+07-20) | ATG GGA TCC AGT ATA CTT ACA GGC TCC | 50 |
| H52A(-01+24) | CTG TTC CAA ATC CTG CAT TGT TGC C | 51 |

wherein A is

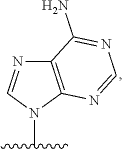 [SMILES: *n1cnc2c(N)ncnc21]

### C is

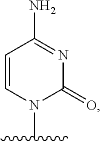 [SMILES: *n1ccc(N)nc1=O]

### G is

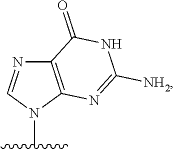 [SMILES: *n1cnc2c(=O)[nH]c(N)nc21]

### T is

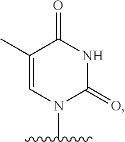 [SMILES: *n1cc(C)c(=O)[nH]c1=O]

### U is

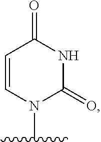 [SMILES: *n1ccc(=O)[nH]c1=O]

Gm is methylated guanine, Am is methylated adenine, and
m5C is

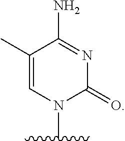 [SMILES: *n1cc(C)c(N)nc1=O]

In certain embodiments, each Nu from 1 to (n+1) and 5′ to 3′ corresponds to SEQ ID NO: 1, SEQ ID NO: 7, or SEQ ID NO: 17.

In various embodiments, R²⁰⁰ is hydrogen. In various embodiments, T′ is

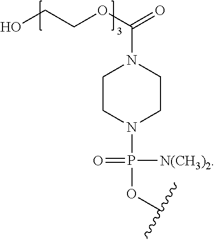

In some embodiments, an antisense oligonucleotide or antisense oligonucleotide conjugate of Formula (I) is in free base form. In some embodiments, an antisense oligonucleotide or antisense oligonucleotide conjugate of Formula (I) is a pharmaceutically acceptable salt thereof. In some embodiments, the antisense oligonucleotide or antisense oligonucleotide conjugate of Formula (I) is in the form of a halide salt. In some embodiments, the antisense oligonucleotide or antisense oligonucleotide conjugate of Formula (I) is in the form of a hexahalide salt form. In some embodiments, an antisense oligonucleotide or antisense oligonucleotide conjugate of Formula (I) is an HCl (hydrochloric acid) salt thereof. In certain embodiments, the HCl salt is a 6HCl salt. In some embodiments, the antisense oligonucleotide or antisense oligonucleotide conjugate of Formula (I) is provided as a mixture of free base and salt form.

In various aspects, the antisense oligonucleotide or antisense oligonucleotide conjugate is according to Formula (II):

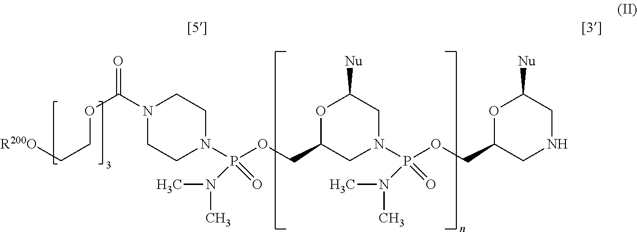

or a pharmaceutically acceptable salt thereof, wherein R²⁰¹ is hydrogen or a cell-penetrating peptide and where each Nu from 1 to (n+1) and 5′ to 3′ corresponds to the nucleobases in one of the following:

| Annealing Site | Targeting Sequence [5′ to 3′] | SEQ ID NO: |
|---|---|---|
| H51A(+66+95) | CTC CAA CAT CAA GGA AGA TGG CAT TTC TAG | 1 |
| H51A(+74+97) | ACC TCC AAC ATC AAG GAA GAT GGC | 2 |
| H51A(+70+99) | GTA CCT CCA ACA TCA AGG AAG ATG GCA TTT | 3 |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |
| H53A(+36+60) | GTT GCC TCC GGT TCT GAA GGT GTT C | 7 |
| H53A(+36+60) | GTT G5mC5mC T5mC5mC GGT T5mC T GAA GGT GTT 5mC | 8 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG TAC | 13 |
| H53A(+39+62) | CTG TTG CCT CCG GTT CTG AAG GTG | 14 |
| H53A(+36+69) | CAT TCA ACT GTT GCC TCC GGT TCT GAA GGT G | 15 |
| H53A(+45+62) | CTG TTG CCT CCG GTT CTG | 16 |
| H45A(-03+19) | CAA TGC CAT CCT GGA GTT CCT G | 17 |
| H45A(-09+25) | GCT GCC CAA TGC CAT CCT GGA GTT CCT GTA AGA T | 18 |
| H45A(-03+25) | GCT GCC CAA TGC CAT CCT GGA GTT CCT G | 19 |
| H45A(-06+25) | GCT GCC CAA TGC CAT CCT GGA GTT CCT GTA A | 20 |
| H45A(-12+19) | CAA TGC CAT CCT GGA GTT CCT GTA AGA TAC C | 21 |
| H45A(-09+19) | CAA TGC CAT CCT GGA GTT CCT GTA AGA T | 22 |
| H45A(-12+16) | TGC CAT CCT GGA GTT CCT GTA AGA TAC C | 23 |
| H45A(-14+25) | GCT GCC CAA TGC CAT CCT GGA GTT CCT GTA AGA | 24 |
| | TAC CAA | |
| H45A(-08+19) | CAA TGC CAT CCT GGA GTT CCT GTA AGA | 25 |
| HM45A(-07+25) | GCT GCC CAA TGC CAT CCT GGA GTT CCT GTA AG | 26 |
| H45A(-12+22) | GCC CAA TGC CAT CCT GGA GTT CCT GTA AGA TAC C | 27 |
| H45A(-09+22) | GCC CAA TGC CAT CCT GGA GTT CCT GTA AGA T | 28 |
| H45A(-09+30) | TTG CCG CTG CCC AAT GCC ATC CTG GAG TTC CTG TAA | 29 |
| | GAT | |
| H45A(-06+22) | GCC CAA TGC CAT CCT GGA GTT CCT GTA A | 30 |
| H45A(-06+28) | GCC GCT GCC CAA TGC CAT CCT GGA GTT CCT GTA A | 31 |
| H45A(-03+22) | GCC CAA TGC CAT CCT GGA GTT CCT G | 32 |
| H45A(-03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A-U-m5C- | 34 |
| | m5C | |
| H44A(-10+15) | GAT CTG TCA AAT CGC CTG CAG GTA A | 35 |
| H44A(-07+15) | GAT CTG TCA AAT CGC CTG CAG G | 36 |
| H44M(-07+17) | CAG ATC TGT CAA ATC GCC TGC AGG | 37 |
| H44A(-08+15) | GAT CTG TCA AAT CGC CTG CAG GT | 38 |
| H44A(-06+15) | GAT CTG TCA AAT CGC CTG CAG | 39 |
| H44A(-08+17) | CAG ATC TGT CAA ATC GCC TGC AGG T | 40 |
| H44A(-06+17) | CAG ATC TGT CAA ATC GCC TGC AG | 41 |
| H50D(+04-18) | GGG ATC CAG TAT ACT TAC AGG C | 42 |
| H50D(+07-18) | GGG ATC CAG TAT ACT TAC AGG CTC C | 43 |
| H50D(+07-16) | GAT CCA GTA TAC TTA CAG GCT CC | 44 |
| H50D(+07-17) | GGA TCC AGT ATA CTT ACA GGC TCC | 45 |
| H50A(-19+07) | ACT TCC TCT TTA ACA GAA AAG CAT AC | 46 |
| H50D(+07-15) | ATC CAG TAT ACT TAC AGG CTC C | 47 |
| H50A(-02+23) | GAG CTC AGA TCT TCT AAC TTC CTC T | 48 |
| H50D(+06-18) | GGG ATC CAG TAT ACT TAC AGG CTC | 49 |
| H50D(+07-20) | ATG GGA TCC AGT ATA CTT ACA GGC TCC | 50 |
| H52A(-01+24) | CTG TTC CAA ATC CTG CAT TGT TGC C | 51 |

wherein A is

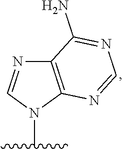 [SMILES: *n1cnc2c(N)ncnc21]

### C is

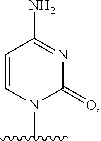 [SMILES: *n1ccc(N)nc1=O]

### G is

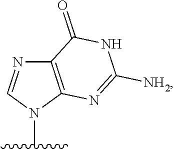 [SMILES: *n1cnc2c(=O)[nH]c(N)nc21]

### T is

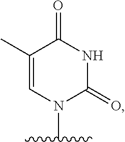 [SMILES: *n1cc(C)c(=O)[nH]c1=O]

### U is

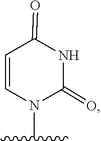 [SMILES: *n1ccc(=O)[nH]c1=O]

Gm is methylated guanine, Am is methylated adenine, and m5C is

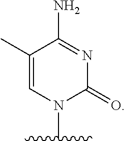 [SMILES: *n1cc(C)c(N)nc1=O]

In certain embodiments, each Nu from 1 to (n+1) and 5′ to 3 corresponds to SEQ ID NO: 1, SEQ ID NO: 7, or SEQ ID NO: 17.

In various embodiments, R²⁰⁰ is hydrogen,

In some embodiments, an antisense oligonucleotide or antisense oligonucleotide conjugate of Formula (II) is in free base form. In some embodiments, an antisense oligonucleotide or antisense oligonucleotide conjugate of Formula (II) is a pharmaceutically acceptable salt thereof. In some embodiments, the antisense oligonucleotide or antisense oligonucleotide conjugate of Formula (II) is in the form of a halide salt. In some embodiments, the antisense oligonucleotide or antisense oligonucleotide conjugate of Formula (II) is in the form of a hexahalide salt form. In some embodiments, an antisense oligonucleotide or antisense oligonucleotide conjugate of Formula (II) is an HCl (hydrochloric acid) salt thereof. In certain embodiments, the HCl salt is a 6HCl salt. In some embodiments, the antisense oligonucleotide or antisense oligonucleotide conjugate of Formula (II) is provided as a mixture of free base and salt form.

In various aspects, the antisense oligonucleotide conjugate is according to Formula (III):

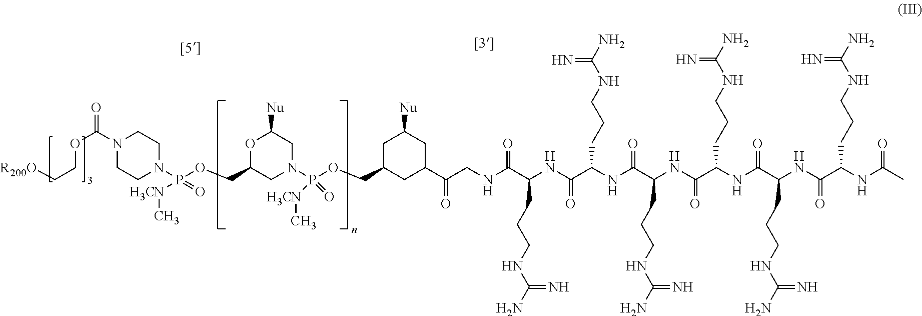

or a pharmaceutically acceptable salt thereof, wherein R²⁰⁰ is hydrogen or a cell-penetrating peptide and wherein each Nu from 1 to (n+1) and 5′ to 3′ corresponds to the nucleobases in one of the following:

| Annealing Site | Targeting Sequence [5′ to 3′] | SEQ ID NO: |
|---|---|---|
| H51A(+66+95) | CTC CAA CAT CAA GGA AGA TGG CAT TTC TAG | 1 |
| H51A(+74+97) | ACC TCC AAC ATC AAG GAA GAT GGC | 2 |
| H51A(+70+99) | GTA CCT CCA ACA TCA AGG AAG ATG GCA TTT | 3 |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |
| H53A(+36+60) | GTT GCC TCC GGT TCT GAA GGT GTT C | 7 |
| H53A(+36+60) | GTT G5mC5mC T5mC5mC GGT T5mC T GAA GGT GTT 5mC | 8 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG TAC | 13 |
| H53A(+39+62) | CTG TTG CCT CCG GTT CTG AAG GTG | 14 |
| H53A(+36+69) | CAT TCA ACT GTT GCC TCC GGT TCT GAA GGT G | 15 |
| H53A(+45+62) | CTG TTG CCT CCG GTT CTG | 16 |
| H45A(-03+19) | CAA TGC CAT CCT GGA GTT CCT G | 17 |
| H45A(-09+25) | GCT GCC CAA TGC CAT CCT GGA GTT CCT GTA AGA T | 18 |
| H45A(-03+25) | GCT GCC CAA TGC CAT CCT GGA GTT CCT G | 19 |
| H45A(-06+25) | GCT GCC CAA TGC CAT CCT GGA GTT CCT GTA A | 20 |
| H45A(-12+19) | CAA TGC CAT CCT GGA GTT CCT GTA AGA TAC C | 21 |
| H45A(-09+19) | CAA TGC CAT CCT GGA GTT CCT GTA AGA T | 22 |
| H45A(-12+16) | TGC CAT CCT GGA GTT CCT GTA AGA TAC C | 23 |
| H45A(-14+25) | GCT GCC CAA TGC CAT CCT GGA GTT CCT GTA AGA | 24 |
| | TAC CAA | |
| H45A(-08+19) | CAA TGC CAT CCT GGA GTT CCT GTA AGA | 25 |
| HM45A(-07+25) | GCT GCC CAA TGC CAT CCT GGA GTT CCT GTA AG | 26 |
| H45A(-12+22) | GCC CAA TGC CAT CCT GGA GTT CCT GTA AGA TAC C | 27 |
| H45A(-09+22) | GCC CAA TGC CAT CCT GGA GTT CCT GTA AGA T | 28 |
| H45A(-09+30) | TTG CCG CTG CCC AAT GCC ATC CTG GAG TTC CTG TAA | 29 |
| | GAT | |
| H45A(-06+22) | GCC CAA TGC CAT CCT GGA GTT CCT GTA A | 30 |
| H45A(-06+28) | GCC GCT GCC CAA TGC CAT CCT GGA GTT CCT GTA A | 31 |
| H45A(-03+22) | GCC CAA TGC CAT CCT GGA GTT CCT G | 32 |
| H45A(-03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A-U-m5C- | 34 |
| | m5C | |
| H44A(-10+15) | GAT CTG TCA AAT CGC CTG CAG GTA A | 35 |
| H44A(-07+15) | GAT CTG TCA AAT CGC CTG CAG G | 36 |
| H44M(-07+17) | CAG ATC TGT CAA ATC GCC TGC AGG | 37 |
| H44A(-08+15) | GAT CTG TCA AAT CGC CTG CAG GT | 38 |
| H44A(-06+15) | GAT CTG TCA AAT CGC CTG CAG | 39 |
| H44A(-08+17) | CAG ATC TGT CAA ATC GCC TGC AGG T | 40 |
| H44A(-06+17) | CAG ATC TGT CAA ATC GCC TGC AG | 41 |
| H50D(+04-18) | GGG ATC CAG TAT ACT TAC AGG C | 42 |
| H50D(+07-18) | GGG ATC CAG TAT ACT TAC AGG CTC C | 43 |
| H50D(+07-16) | GAT CCA GTA TAC TTA CAG GCT CC | 44 |
| H50D(+07-17) | GGA TCC AGT ATA CTT ACA GGC TCC | 45 |
| H50A(-19+07) | ACT TCC TCT TTA ACA GAA AAG CAT AC | 46 |
| H50D(+07-15) | ATC CAG TAT ACT TAC AGG CTC C | 47 |
| H50A(-02+23) | GAG CTC AGA TCT TCT AAC TTC CTC T | 48 |
| H50D(+06-18) | GGG ATC CAG TAT ACT TAC AGG CTC | 49 |
| H50D(+07-20) | ATG GGA TCC AGT ATA CTT ACA GGC TCC | 50 |
| H52A(-01+24) | CTG TTC CAA ATC CTG CAT TGT TGC C | 51 |

wherein A is

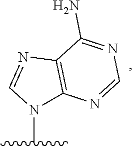 [SMILES: *n1cnc2c(N)ncnc21]

### C is

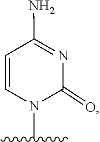 [SMILES: *n1ccc(N)nc1=O]

### G is

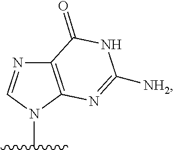 [SMILES: *n1cnc2c(=O)[nH]c(N)nc21]

### T is

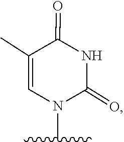 [SMILES: *n1cc(C)c(=O)[nH]c1=O]

### U is

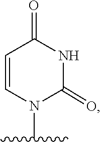 [SMILES: *n1ccc(=O)[nH]c1=O]

Gm is methylated guanine, Am is methylated adenine, and m5C is

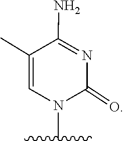 [SMILES: *n1cc(C)c(N)nc1=O]

In certain embodiments, each Nu from 1 to (n+1) and 5′ to 3′ corresponds to SEQ ID NO: 1, SEQ ID NO: 7, or SEQ ID NO: 17.

In various embodiments, R²⁰⁰ is hydrogen.

In some embodiments, an antisense oligonucleotide conjugate of Formula (III) is in free base form. In some embodiments, an antisense oligonucleotide conjugate of Formula (III) is a pharmaceutically acceptable salt thereof. In some embodiments, the antisense oligonucleotide or antisense oligonucleotide conjugate of Formula (III) is in the form of a halide salt. In some embodiments, the antisense oligonucleotide or antisense oligonucleotide conjugate of Formula (III) is in the form of a hexahalide salt form. In some embodiments, an antisense oligonucleotide conjugate of Formula (III) is an HCl (hydrochloric acid) salt thereof. In certain embodiments, the HCl salt is a 6HCl salt. In some embodiments, the antisense oligonucleotide or antisense oligonucleotide conjugate of Formula (III) is provided as a mixture of free base and salt form.

In various aspects, the antisense oligonucleotide conjugate is according to Formula (IV):

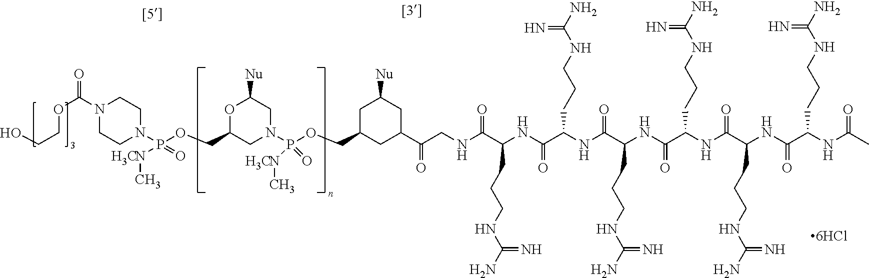

where each Nu from 1 to (n+1) and 5′ to 3′ corresponds to the nucleobases in one of the following:

| Annealing Site | Targeting Sequence [5′ to 3′] | SEQ ID NO: |
|---|---|---|
| H51A(+66+95) | CTC CAA CAT CAA GGA AGA TGG CAT TTC TAG | 1 |
| H51A(+74+97) | ACC TCC AAC ATC AAG GAA GAT GGC | 2 |
| H51A(+70+99) | GTA CCT CCA ACA TCA AGG AAG ATG GCA TTT | 3 |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |
| H53A(+36+60) | GTT GCC TCC GGT TCT GAA GGT GTT C | 7 |
| H53A(+36+60) | GTT G5mC5mC T5mC5mC GGT T5mC T GAA GGT GTT 5mC | 8 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG TAC | 13 |
| H53A(+39+62) | CTG TTG CCT CCG GTT CTG AAG GTG | 14 |
| H53A(+36+69) | CAT TCA ACT GTT GCC TCC GGT TCT GAA GGT G | 15 |
| H53A(+45+62) | CTG TTG CCT CCG GTT CTG | 16 |
| H45A(-03+19) | CAA TGC CAT CCT GGA GTT CCT G | 17 |
| H45A(-09+25) | GCT GCC CAA TGC CAT CCT GGA GTT CCT GTA AGA T | 18 |
| H45A(-03+25) | GCT GCC CAA TGC CAT CCT GGA GTT CCT G | 19 |
| H45A(-06+25) | GCT GCC CAA TGC CAT CCT GGA GTT CCT GTA A | 20 |
| H45A(-12+19) | CAA TGC CAT CCT GGA GTT CCT GTA AGA TAC C | 21 |
| H45A(-09+19) | CAA TGC CAT CCT GGA GTT CCT GTA AGA T | 22 |
| H45A(-12+16) | TGC CAT CCT GGA GTT CCT GTA AGA TAC C | 23 |
| H45A(-14+25) | GCT GCC CAA TGC CAT CCT GGA GTT CCT GTA AGA | 24 |
| | TAC CAA | |
| H45A(-08+19) | CAA TGC CAT CCT GGA GTT CCT GTA AGA | 25 |
| HM45A(-07+25) | GCT GCC CAA TGC CAT CCT GGA GTT CCT GTA AG | 26 |
| H45A(-12+22) | GCC CAA TGC CAT CCT GGA GTT CCT GTA AGA TAC C | 27 |
| H45A(-09+22) | GCC CAA TGC CAT CCT GGA GTT CCT GTA AGA T | 28 |
| H45A(-09+30) | TTG CCG CTG CCC AAT GCC ATC CTG GAG TTC CTG TAA | 29 |
| | GAT | |
| H45A(-06+22) | GCC CAA TGC CAT CCT GGA GTT CCT GTA A | 30 |
| H45A(-06+28) | GCC GCT GCC CAA TGC CAT CCT GGA GTT CCT GTA A | 31 |
| H45A(-03+22) | GCC CAA TGC CAT CCT GGA GTT CCT G | 32 |
| H45A(-03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A-U-m5C- | 34 |
| | m5C | |
| H44A(-10+15) | GAT CTG TCA AAT CGC CTG CAG GTA A | 35 |
| H44A(-07+15) | GAT CTG TCA AAT CGC CTG CAG G | 36 |
| H44M(-07+17) | CAG ATC TGT CAA ATC GCC TGC AGG | 37 |
| H44A(-08+15) | GAT CTG TCA AAT CGC CTG CAG GT | 38 |
| H44A(-06+15) | GAT CTG TCA AAT CGC CTG CAG | 39 |
| H44A(-08+17) | CAG ATC TGT CAA ATC GCC TGC AGG T | 40 |
| H44A(-06+17) | CAG ATC TGT CAA ATC GCC TGC AG | 41 |
| H50D(+04-18) | GGG ATC CAG TAT ACT TAC AGG C | 42 |
| H50D(+07-18) | GGG ATC CAG TAT ACT TAC AGG CTC C | 43 |
| H50D(+07-16) | GAT CCA GTA TAC TTA CAG GCT CC | 44 |
| H50D(+07-17) | GGA TCC AGT ATA CTT ACA GGC TCC | 45 |
| H50A(-19+07) | ACT TCC TCT TTA ACA GAA AAG CAT AC | 46 |
| H50D(+07-15) | ATC CAG TAT ACT TAC AGG CTC C | 47 |
| H50A(-02+23) | GAG CTC AGA TCT TCT AAC TTC CTC T | 48 |
| H50D(+06-18) | GGG ATC CAG TAT ACT TAC AGG CTC | 49 |
| H50D(+07-20) | ATG GGA TCC AGT ATA CTT ACA GGC TCC | 50 |
| H52A(-01+24) | CTG TTC CAA ATC CTG CAT TGT TGC C | 51 |

wherein A is

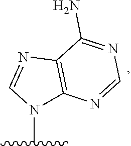 [SMILES: *n1cnc2c(N)ncnc21]

### C is

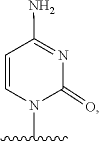 [SMILES: *n1ccc(N)nc1=O]

### G is

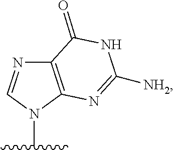 [SMILES: *n1cnc2c(=O)[nH]c(N)nc21]

### T is

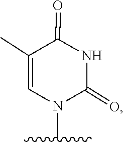 [SMILES: *n1cc(C)c(=O)[nH]c1=O]

### U is

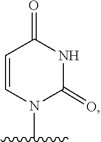 [SMILES: *n1ccc(=O)[nH]c1=O]

Gm is methylated guanine, Am is methylated adenine, and m5C is

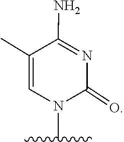 [SMILES: *n1cc(C)c(N)nc1=O]

In certain embodiments, each Nu from 1 to (n+1) and 5′ to 3′ corresponds to SEQ ID NO: 1, SEQ ID NO: 7, or SEQ ID NO: 17.

In various aspects, the antisense oligonucleotide conjugate is according to Formula (V):

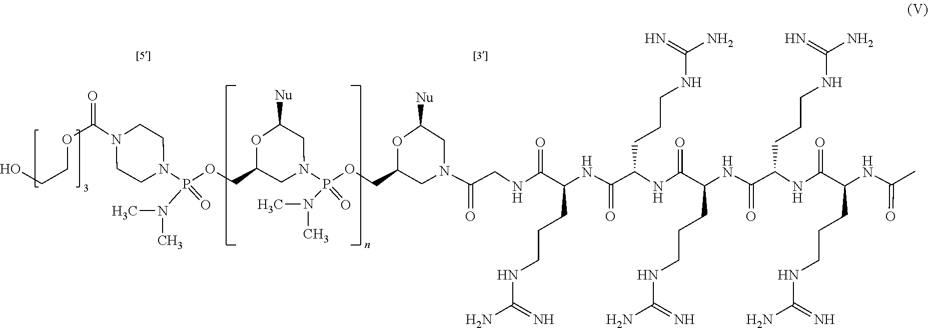

or a pharmaceutically acceptable salt thereof, where each Nu from 1 to (n+1) and 5′ to 3′ corresponds to the nucleobases in one of the following:

| Annealing Site | Targeting Sequence [5′ to 3′] | SEQ ID NO: |
|---|---|---|
| H51A(+66+95) | CTC CAA CAT CAA GGA AGA TGG CAT TTC TAG | 1 |
| H51A(+74+97) | ACC TCC AAC ATC AAG GAA GAT GGC | 2 |
| H51A(+70+99) | GTA CCT CCA ACA TCA AGG AAG ATG GCA TTT | 3 |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |
| H53A(+36+60) | GTT GCC TCC GGT TCT GAA GGT GTT C | 7 |
| H53A(+36+60) | GTT G5mC5mC T5mC5mC GGT T5mC T GAA GGT GTT 5mC | 8 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG TAC | 13 |
| H53A(+39+62) | CTG TTG CCT CCG GTT CTG AAG GTG | 14 |
| H53A(+36+69) | CAT TCA ACT GTT GCC TCC GGT TCT GAA GGT G | 15 |
| H53A(+45+62) | CTG TTG CCT CCG GTT CTG | 16 |
| H45A(-03+19) | CAA TGC CAT CCT GGA GTT CCT G | 17 |
| H45A(-09+25) | GCT GCC CAA TGC CAT CCT GGA GTT CCT GTA AGA T | 18 |
| H45A(-03+25) | GCT GCC CAA TGC CAT CCT GGA GTT CCT G | 19 |
| H45A(-06+25) | GCT GCC CAA TGC CAT CCT GGA GTT CCT GTA A | 20 |
| H45A(-12+19) | CAA TGC CAT CCT GGA GTT CCT GTA AGA TAC C | 21 |
| H45A(-09+19) | CAA TGC CAT CCT GGA GTT CCT GTA AGA T | 22 |
| H45A(-12+16) | TGC CAT CCT GGA GTT CCT GTA AGA TAC C | 23 |
| H45A(-14+25) | GCT GCC CAA TGC CAT CCT GGA GTT CCT GTA AGA | 24 |
| | TAC CAA | |
| H45A(-08+19) | CAA TGC CAT CCT GGA GTT CCT GTA AGA | 25 |
| HM45A(-07+25) | GCT GCC CAA TGC CAT CCT GGA GTT CCT GTA AG | 26 |
| H45A(-12+22) | GCC CAA TGC CAT CCT GGA GTT CCT GTA AGA TAC C | 27 |
| H45A(-09+22) | GCC CAA TGC CAT CCT GGA GTT CCT GTA AGA T | 28 |
| H45A(-09+30) | TTG CCG CTG CCC AAT GCC ATC CTG GAG TTC CTG TAA | 29 |
| | GAT | |
| H45A(-06+22) | GCC CAA TGC CAT CCT GGA GTT CCT GTA A | 30 |
| H45A(-06+28) | GCC GCT GCC CAA TGC CAT CCT GGA GTT CCT GTA A | 31 |
| H45A(-03+22) | GCC CAA TGC CAT CCT GGA GTT CCT G | 32 |
| H45A(-03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A-U-m5C- | 34 |
| | m5C | |
| H44A(-10+15) | GAT CTG TCA AAT CGC CTG CAG GTA A | 35 |
| H44A(-07+15) | GAT CTG TCA AAT CGC CTG CAG G | 36 |
| H44M(-07+17) | CAG ATC TGT CAA ATC GCC TGC AGG | 37 |
| H44A(-08+15) | GAT CTG TCA AAT CGC CTG CAG GT | 38 |
| H44A(-06+15) | GAT CTG TCA AAT CGC CTG CAG | 39 |
| H44A(-08+17) | CAG ATC TGT CAA ATC GCC TGC AGG T | 40 |
| H44A(-06+17) | CAG ATC TGT CAA ATC GCC TGC AG | 41 |
| H50D(+04-18) | GGG ATC CAG TAT ACT TAC AGG C | 42 |
| H50D(+07-18) | GGG ATC CAG TAT ACT TAC AGG CTC C | 43 |
| H50D(+07-16) | GAT CCA GTA TAC TTA CAG GCT CC | 44 |
| H50D(+07-17) | GGA TCC AGT ATA CTT ACA GGC TCC | 45 |
| H50A(-19+07) | ACT TCC TCT TTA ACA GAA AAG CAT AC | 46 |
| H50D(+07-15) | ATC CAG TAT ACT TAC AGG CTC C | 47 |
| H50A(-02+23) | GAG CTC AGA TCT TCT AAC TTC CTC T | 48 |
| H50D(+06-18) | GGG ATC CAG TAT ACT TAC AGG CTC | 49 |
| H50D(+07-20) | ATG GGA TCC AGT ATA CTT ACA GGC TCC | 50 |
| H52A(-01+24) | CTG TTC CAA ATC CTG CAT TGT TGC C | 51 |

wherein A is

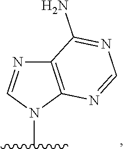 [SMILES: *n1cnc2c(N)ncnc21]

### C is

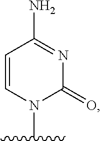 [SMILES: *n1ccc(N)nc1=O]

### G is

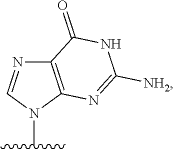 [SMILES: *n1cnc2c(=O)[nH]c(N)nc21]

### T is

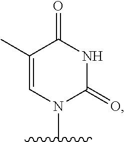 [SMILES: *n1cc(C)c(=O)[nH]c1=O]

### U is

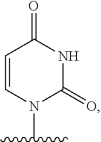 [SMILES: *n1ccc(=O)[nH]c1=O]

Gm is methylated guanine, Am is methylated adenine, and m5C is

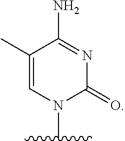 [SMILES: *n1cc(C)c(N)nc1=O]

In certain embodiments, each Nu from 1 to (n+1) and 5′ to 3′ corresponds to SEQ ID NO: 1, SEQ ID NO: 7, or SEQ ID NO: 17.

In various embodiments, R²⁰⁰ is hydrogen.

In some embodiments, an antisense oligonucleotide conjugate of Formula (V) is in free base form. In some embodiments, an antisense oligonucleotide conjugate of Formula (V) is a pharmaceutically acceptable salt thereof. In some embodiments, the antisense oligonucleotide or antisense oligonucleotide conjugate of Formula (V) is in the form of a halide salt. In some embodiments, the antisense oligonucleotide or antisense oligonucleotide conjugate of Formula (V) is in the form of a pentahalide salt form. In some embodiments, an antisense oligonucleotide conjugate of Formula (V) is an HCl (hydrochloric acid) salt thereof. In certain embodiments, the HCl salt is a 5HCl salt. In some embodiments, the antisense oligonucleotide or antisense oligonucleotide conjugate of Formula (V) is provided as a mixture of free base and salt form.

In various aspects, the antisense oligonucleotide conjugate is according to Formula (VI):

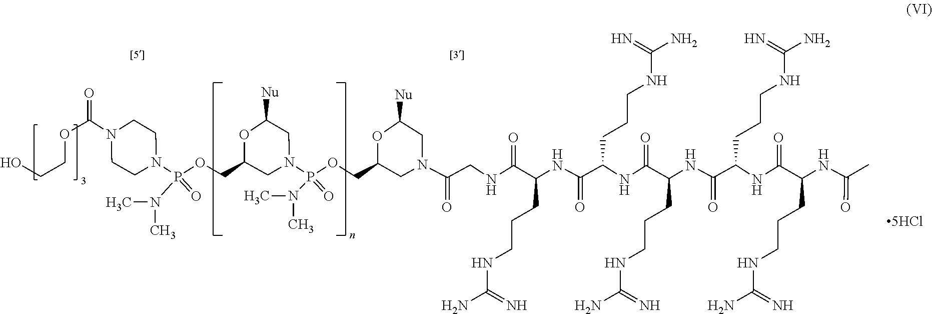

where each Nu from 1 to (n+1) and 5′ to 3′ corresponds to the nucleobases in one of the following:

| Annealing Site | Targeting Sequence [5′ to 3′] | SEQ ID NO: |
|---|---|---|
| H51A(+66+95) | CTC CAA CAT CAA GGA AGA TGG CAT TTC TAG | 1 |
| H51A(+74+97) | ACC TCC AAC ATC AAG GAA GAT GGC | 2 |
| H51A(+70+99) | GTA CCT CCA ACA TCA AGG AAG ATG GCA TTT | 3 |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |
| H53A(+36+60) | GTT GCC TCC GGT TCT GAA GGT GTT C | 7 |
| H53A(+36+60) | GTT G5mC5mC T5mC5mC GGT T5mC T GAA GGT GTT 5mC | 8 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG TAC | 13 |
| H53A(+39+62) | CTG TTG CCT CCG GTT CTG AAG GTG | 14 |
| H53A(+36+69) | CAT TCA ACT GTT GCC TCC GGT TCT GAA GGT G | 15 |
| H53A(+45+62) | CTG TTG CCT CCG GTT CTG | 16 |
| H45A(-03+19) | CAA TGC CAT CCT GGA GTT CCT G | 17 |
| H45A(-09+25) | GCT GCC CAA TGC CAT CCT GGA GTT CCT GTA AGA T | 18 |
| H45A(-03+25) | GCT GCC CAA TGC CAT CCT GGA GTT CCT G | 19 |
| H45A(-06+25) | GCT GCC CAA TGC CAT CCT GGA GTT CCT GTA A | 20 |
| H45A(-12+19) | CAA TGC CAT CCT GGA GTT CCT GTA AGA TAC C | 21 |
| H45A(-09+19) | CAA TGC CAT CCT GGA GTT CCT GTA AGA T | 22 |
| H45A(-12+16) | TGC CAT CCT GGA GTT CCT GTA AGA TAC C | 23 |
| H45A(-14+25) | GCT GCC CAA TGC CAT CCT GGA GTT CCT GTA AGA | 24 |
| | TAC CAA | |
| H45A(-08+19) | CAA TGC CAT CCT GGA GTT CCT GTA AGA | 25 |
| HM45A(-07+25) | GCT GCC CAA TGC CAT CCT GGA GTT CCT GTA AG | 26 |
| H45A(-12+22) | GCC CAA TGC CAT CCT GGA GTT CCT GTA AGA TAC C | 27 |
| H45A(-09+22) | GCC CAA TGC CAT CCT GGA GTT CCT GTA AGA T | 28 |
| H45A(-09+30) | TTG CCG CTG CCC AAT GCC ATC CTG GAG TTC CTG TAA | 29 |
| | GAT | |
| H45A(-06+22) | GCC CAA TGC CAT CCT GGA GTT CCT GTA A | 30 |
| H45A(-06+28) | GCC GCT GCC CAA TGC CAT CCT GGA GTT CCT GTA A | 31 |
| H45A(-03+22) | GCC CAA TGC CAT CCT GGA GTT CCT G | 32 |
| H45A(-03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A-U-m5C- | 34 |
| | m5C | |
| H44A(-10+15) | GAT CTG TCA AAT CGC CTG CAG GTA A | 35 |
| H44A(-07+15) | GAT CTG TCA AAT CGC CTG CAG G | 36 |
| H44M(-07+17) | CAG ATC TGT CAA ATC GCC TGC AGG | 37 |
| H44A(-08+15) | GAT CTG TCA AAT CGC CTG CAG GT | 38 |
| H44A(-06+15) | GAT CTG TCA AAT CGC CTG CAG | 39 |
| H44A(-08+17) | CAG ATC TGT CAA ATC GCC TGC AGG T | 40 |
| H44A(-06+17) | CAG ATC TGT CAA ATC GCC TGC AG | 41 |
| H50D(+04-18) | GGG ATC CAG TAT ACT TAC AGG C | 42 |
| H50D(+07-18) | GGG ATC CAG TAT ACT TAC AGG CTC C | 43 |
| H50D(+07-16) | GAT CCA GTA TAC TTA CAG GCT CC | 44 |
| H50D(+07-17) | GGA TCC AGT ATA CTT ACA GGC TCC | 45 |
| H50A(-19+07) | ACT TCC TCT TTA ACA GAA AAG CAT AC | 46 |
| H50D(+07-15) | ATC CAG TAT ACT TAC AGG CTC C | 47 |
| H50A(-02+23) | GAG CTC AGA TCT TCT AAC TTC CTC T | 48 |
| H50D(+06-18) | GGG ATC CAG TAT ACT TAC AGG CTC | 49 |
| H50D(+07-20) | ATG GGA TCC AGT ATA CTT ACA GGC TCC | 50 |
| H52A(-01+24) | CTG TTC CAA ATC CTG CAT TGT TGC C | 51 |

wherein A is

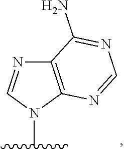 [SMILES: *n1cnc2c(N)ncnc21]

### C is

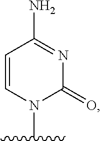 [SMILES: *n1ccc(N)nc1=O]

### G is

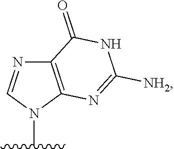 [SMILES: *n1cnc2c(=O)[nH]c(N)nc21]

### T is

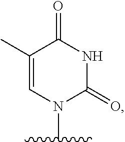 [SMILES: *n1cc(C)c(=O)[nH]c1=O]

### U is

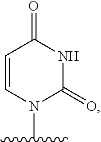 [SMILES: *n1ccc(=O)[nH]c1=O]

Gm is methylated guanine, Am is methylated adenine, and m5C is

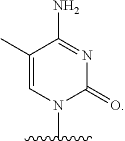 [SMILES: *n1cc(C)c(N)nc1=O]

In certain embodiments, each Nu from 1 to (n+1) and 5′ to 3′ corresponds to SEQ ID NO: 1, SEQ ID NO: 7, or SEQ ID NO: 17.

In certain embodiments, the antisense oligonucleotide or antisense oligonucleotide conjugate, or pharmaceutically acceptable salt thereof, is selected from the group consisting of eteplirsen, PPMO #1, or pharmaceutically acceptable salt thereof, golodirsen, PPMO #2, or pharmaceutically acceptable salt thereof, casimersen, and PPMO #3, or pharmaceutically acceptable salt thereof.

### J. Nucleobase Modifications and Substitutions

In certain embodiments, antisense oligomers of the disclosure are composed of RNA nucleobases and DNA nucleobases (often referred to in the art simply as “base”). RNA bases are commonly known as adenine (A), uracil (U), cytosine (C) and guanine (G). DNA bases are commonly known as adenine (A), thymine (T), cytosine (C) and guanine (G). In various embodiments, antisense oligomers of the disclosure are composed of cytosine (C), guanine (G), thymine (T), adenine (A), 5-methylcytosine (5mC), uracil (U), hypoxanthine (I) methylated guanine (Gm), and methylated adenine (Am).

In certain embodiments, one or more RNA bases or DNA bases in an oligomer may be modified or substituted with a base other than a RNA base or DNA base. Oligomers containing a modified or substituted base include oligomers in which one or more purine or pyrimidine bases most commonly found in nucleic acids are replaced with less common or non-natural bases.

Purine bases comprise a pyrimidine ring fused to an imidazole ring, as described by the following general formula.

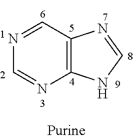

Adenine and guanine are the two purine nucleobases most commonly found in nucleic acids. Other naturally-occurring purines include, but not limited to, N⁶-methyladenine, N²-methylguanine, hypoxanthine, and 7-methylguanine.

Pyrimidine bases comprise a six-membered pyrimidine ring as described by the following general formula.

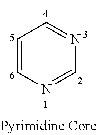

Cytosine, uracil, and thymine are the pyrimidine bases most commonly found in nucleic acids. Other naturally-occurring pyrimidines include, but not limited to, 5-methylcytosine, 5-hydroxymethylcytosine, pseudouracil, and 4-thiouracil. In one embodiment, the oligomers described herein contain thymine bases in place of uracil.

Other suitable bases include, but are not limited to: 2,6-diaminopurine, orotic acid, agmatidine, lysidine, 2-thiopyrimidines (e.g. 2-thiouracil, 2-thiothymine), G-clamp and its derivatives, 5-substituted pyrimidines (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N²-cyclopentylguanine (cPent-G), N²-cyclopentyl-2-aminopurine (cPent-AP), and N²-propyl-2-aminopurine (Pr-AP), pseudouracil, or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Examples of derivatives of Super A, Super G, and Super T can be found in U.S. Pat. No. 6,683,173 (Epoch Biosciences), which is incorporated here entirely by reference. cPent-G, cPent-AP, and Pr-AP were shown to reduce immunostimulatory effects when incorporated in siRNA (Peacock H. et al. J. Am. Chem. Soc. 2011, 133, 9200). Pseudouracil is a naturally occurring isomerized version of uracil, with a C-glycoside rather than the regular N-glycoside as in uridine. Pseudouridine-containing synthetic mRNA may have an improved safety profile compared to uridine-containing mPvNA (WO 2009127230, incorporated here in its entirety by reference).

Certain nucleobases are particularly useful for increasing the binding affinity of the antisense oligomers of the disclosure. These include 5-substituted pyrimidines, 6-azapyrimidines, and N-2, N-6, and O-6 substituted purines, including 2-aminopropyladenine, 5-propynyluracil, and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications. Additional exemplary modified nucleobases include those wherein at least one hydrogen atom of the nucleobase is replaced with fluorine.

### Cell-Penetrating Peptides

In certain embodiments, the antisense oligonucleotide is conjugated to one or more cell-penetrating peptides (referred to herein as “CPP”). In certain embodiments, one or more CPPs are attached to a terminus of the antisense oligonucleotide. In certain embodiments, at least one CPP is attached to the 5′ terminus of the antisense oligonucleotide. In certain embodiments, at least one CPP is attached to the 3′ terminus of the antisense oligonucleotide. In certain embodiments, a first CPP is attached to the 5′ terminus and a second CPP is attached to the 3′ terminus of the antisense oligonucleotide. In certain embodiments, one CPP is attached to the 3′ terminus of the antisense oligonucleotide.

In some embodiments, the CPP is an arginine-rich peptide. The term “arginine-rich” refers to a CPP having at least 2, and preferably 2, 3, 4, 5, 6, 7, or 8 arginine residues, each optionally separated by one or more uncharged, hydrophobic residues, and optionally containing about 6-14 amino acid residues. As explained below, a CPP is preferably linked at its carboxy terminus to the 3′ and/or 5′ end of an antisense oligonucleotide through a linker, which may also be one or more amino acids, and is preferably also capped at its amino terminus by a substituent R^{a} with R^{a} selected from H, acyl, acetyl, benzoyl, or stearoyl. In some embodiments, R^{a} is acetyl.

As seen in the table below, non-limiting examples of CPP's for use herein include —(RXR)₄—R^{a} (SEQ ID NO: 52), R—(FFR)₃—R^{a} (SEQ ID NO: 53), —B—X—(RXR)₄—R^{a} (SEQ ID NO: 54), —B—X—R—(FFR)₃—R^{a} (SEQ ID NO: 55), -GLY-R—(FFR)₃—R^{a} (SEQ ID NO: 56), -GLY-R₆—R^{a} (SEQ ID NO: 57) and —R₆—R^{a} (SEQ ID NO: 58), wherein R^{a} is selected from H, acyl, benzoyl, and stearoyl, and wherein R is arginine, X is 6-aminohexanoic acid, B is β-alanine, F is phenylalanine and GLY (or G) is glycine. The CPP “R₆” (SEQ ID NO: 58) is meant to indicate a peptide of six (6) arginine residues (SEQ ID NO: 58) linked together via amide bonds (and not a single substituent e.g. R₆ (SEQ ID NO: 58)). In some embodiments, R^{a} is acetyl.

Exemplary CPPs are provided in Table 1 (SEQ ID NOS: 52-58).

TABLE 8
Exemplary Cell-Penetrating Peptides

| Name | Sequence | SEQ ID NO: |
|---|---|---|
| (RXR)₄ | RXRRXRRXRRXR | 52 |
| (RFF)₃R | RFFRFFRFFR | 53 |
| (RXR)₄XB | RXRRXRRXRRXRXB | 54 |
| (RFF)₃RXB | RFFRFFRFFRXB | 55 |
| (RFF)₃RG | RFFRFFRFFRG | 56 |
| R₆G | RRRRRRG | 57 |
| R₆ | RRRRRR | 58 |

R is arginine; X is 6-aminohexanoic acid; B is β-alanine; F is phenylalanine; G is glycine

CPPs, their synthesis, and methods of conjugating to an oligomer are further described in U.S. Application Publication No. 2012/0289457 and International Patent Application Publication Nos. WO 2004/097017, WO 2009/005793, and WO 2012/150960, the disclosures of which are incorporated herein by reference in their entirety.

In some embodiments, an antisense oligonucleotide comprises a substituent “Z,” defined as the combination of a CPP and a linker. The linker bridges the CPP at its carboxy terminus to the 3′-end and/or the 5′-end of the oligonucleotide. In various embodiments, an antisense oligonucleotide may comprise only one CPP linked to the 3′ end of the oligomer. In other embodiments, an antisense oligonucleotide may comprise only one CPP linked to the 5′ end of the oligomer.

The linker within Z may comprise, for example, 1, 2, 3, 4, or 5 amino acids.

In particular embodiments, Z is selected from:

—C(O)(CH₂)₅NH—CPP;
—C(O)(CH₂)₂NH—CPP;
—C(O)(CH₂)₂NHC(O)(CH₂)₅NH—CPP;
—C(O)CH₂NH—CPP, and the formula:

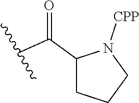

wherein the CPP is attached to the linker moiety by an amide bond at the CPP carboxy terminus.

In various embodiments, the CPP is an arginine-rich peptide as defined above and seen in Table 1. In certain embodiments, the arginine-rich CPP is —R₆—R^{a}, (i.e., six arginine residues; SEQ ID NO: 58), wherein R^{a} is selected from H, acyl, acetyl, benzoyl, and stearoyl. In certain embodiments, R^{a} is acetyl. In various embodiments, the CPP is selected from (RXR)₄ (SEQ ID NOS: 52), (RFF)₃R (SEQ ID NO: 53), or R₆ (SEQ ID NO: 58), and the linker is selected from the group described above. In some embodiments, the CPP is R₆ (SEQ ID NO: 58) and the linker is Gly. In some embodiments, the CPP is R₆G (SEQ ID NO: 57).

In certain embodiments, Z is —C(O)CH₂NH—R₆—R^{a} (“R₆” disclosed as SEQ ID NO: 58) covalently bonded to an antisense oligomer of the disclosure at the 5′ and/or 3′ end of the oligomer, wherein R^{a} is H, acyl, acetyl, benzoyl, or stearoyl to cap the amino terminus of the R₆ (SEQ ID NO: 58). In certain embodiments, R^{a} is acetyl. In these non-limiting examples, the CPP is —R₆—R^{a} (SEQ ID NO: 58) and the linker is —C(O)CH₂NH—, (i.e. GLY). This particular example of Z═—C(O)CH₂NH—R₆—R^{a} (“R₆” disclosed as SEQ ID NO: 58) is also exemplified by the following structure:

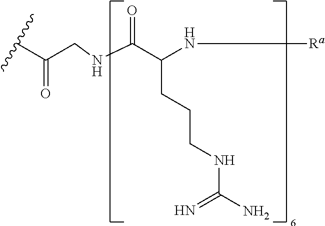

wherein R^{a} is selected from H, acyl, acetyl, benzoyl, and stearoyl.

In various embodiments, the CPP is —R₆—R^{a} (SEQ ID NO: 58), also exemplified as the following formula:

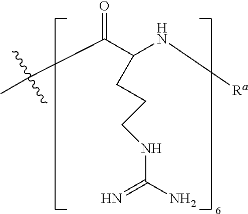

wherein R^{a} is selected from H, acyl, acetyl, benzoyl, and stearoyl. In certain embodiments, the CPP is R₆ (SEQ ID NO: 58). In some embodiments, R^{a} is acetyl.

In some embodiments, the CPP is —(RXR)₄—R^{a} (SEQ ID NO: 52), also exemplified as the following formula:

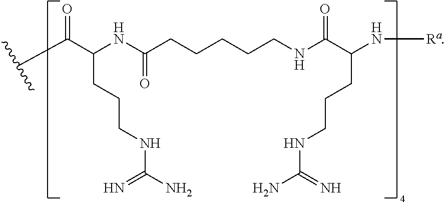

In various embodiments, the CPP is —R—(FFR)₃—R^{a} (SEQ ID NO: 53), also exemplified as the following formula:

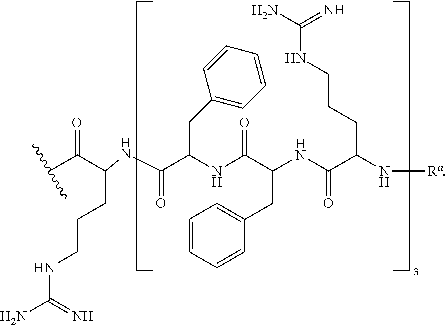

In various embodiments, Z is selected from:

—C(O)(CH₂)₅NH—CPP;
—C(O)(CH₂)₂NH—CPP;
—C(O)(CH₂)₂NHC(O)(CH₂)₅NH—CPP;
—C(O)CH₂NH—CPP; and the formula:

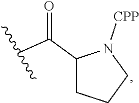

wherein the CPP is attached to the linker moiety by an amide bond at the CPP carboxy terminus, and wherein the CPP is selected from:

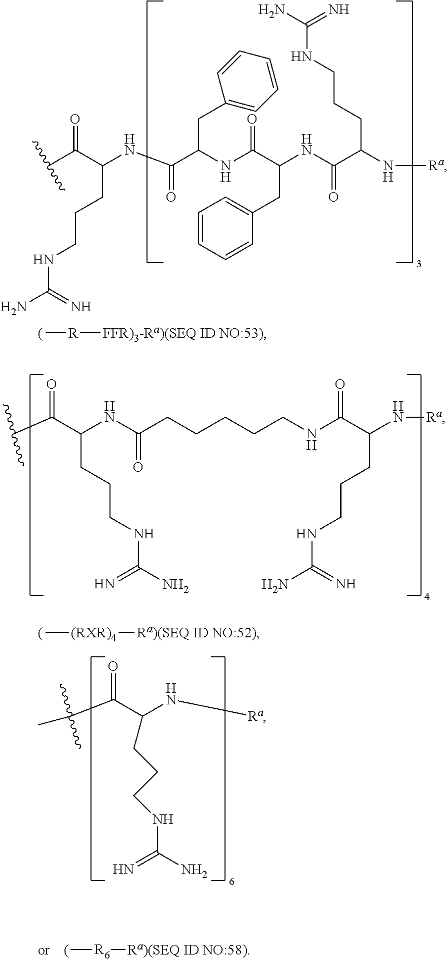

In some embodiments, R^{a} is acetyl.

### Pharmaceutically Acceptable Salts of Antisense Oligonucleotides or Antisense Oligonucleotide Conjugates

Certain embodiments of antisense oligonucleotide conjugates described herein may contain a basic functional group, such as amino or alkylamino, and are, thus, capable of forming pharmaceutically-acceptable salts with pharmaceutically-acceptable acids.

The term “pharmaceutically-acceptable salts” in this respect, refers to the relatively non-toxic, inorganic and organic acid addition salts of antisense oligonucleotides or antisense oligonucleotide conjugates. These salts can be prepared in situ in the administration vehicle or the dosage form manufacturing process, or by separately reacting a purified antisense oligonucleotide conjugate in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, tosylate, citrate, maleate, fumarate, succinate, tartrate, naphthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of the antisense oligonucleotide conjugates include the conventional nontoxic salts or quaternary ammonium salts of the antisense oligonucleotides or antisense oligonucleotide conjugates, e.g., from non-toxic organic or inorganic acids. For example, such conventional nontoxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In certain embodiments, the antisense oligonucleotides or antisense oligonucleotide conjugates may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of antisense oligonucleotide conjugates. These salts can likewise be prepared in situ in the administration vehicle or the dosage form manufacturing process, or by separately reacting the purified antisense oligonucleotide conjugate in its free acid form with a suitable base, such as the hydroxide, carbonate, or bicarbonate of a pharmaceutically-acceptable metal cation, with ammonia, or with a pharmaceutically-acceptable organic primary, secondary, or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., Berge et al., supra).

The salt form can be a complex of multiple cations or anions with the antisense oligomer conjugate. For example, the salt form can be a monohalide, dihalide, trihalide, tetrahalide, pentahalide, or hexahalide.

### IV. FORMULATIONS AND TREATMENT

Formulations or compositions suitable for the therapeutic delivery comprise an effective amount of an antisense oligonucleotide or antisense oligonucleotide conjugate, or pharmaceutically acceptable salt thereof, formulated together with one or more pharmaceutically acceptable carriers (additives) and/or diluents. While it is possible for an antisense oligonucleotide or antisense oligonucleotide conjugate, or pharmaceutically acceptable salt thereof, to be administered alone, it is preferable to administer the compound as a pharmaceutical formulation (composition).

In some embodiments, the antisense oligonucleotide or antisense oligonucleotide conjugate, is provided in a pharmaceutical composition formed by dissolving 0.005 mg/kg to about 300 mg/kg of the antisense oligonucleotide conjugate, or pharmaceutically acceptable salt thereof, in an aqueous carrier solution. In some aspects, the aqueous carrier solution is sterile water or saline.

In some aspects, the human patient is administered the antisense oligonucleotide or antisense oligonucleotide conjugate, or pharmaceutically acceptable salt thereof, at least six months, at least one year, at least two years, at least three years, at least four years, or at least five years.

The compositions may be administered alone or in combination with another therapeutic. The additional therapeutic may be administered prior, concurrently or subsequently to the administration of the composition comprising an antisense oligonucleotide or antisense oligonucleotide conjugate, or pharmaceutically acceptable salt thereof. For example, the compositions may be administered in combination with a steroid and/or an antibiotic. The steroid may be a glucocorticoid or prednisone. Glucocorticoids such as cortisol control carbohydrate, fat and protein metabolism, and are anti-inflammatory by preventing phospholipid release, decreasing eosinophil action and a number of other mechanisms. Mineralocorticoids such as aldosterone control electrolyte and water levels, mainly by promoting sodium retention in the kidney. Corticosteroids are a class of chemicals that includes steroid hormones naturally produced in the adrenal cortex of vertebrates and analogues of these hormones that are synthesized in laboratories. Corticosteroids are involved in a wide range of physiological processes, including stress response, immune response, and regulation of inflammation, carbohydrate metabolism, protein catabolism, blood electrolyte levels, and behavior. Corticosteroids include Betamethasone, Budesonide, Cortisone, Dexamethasone, Hydrocortisone, Methylprednisolone, Prednisolone, and Prednisone.

Other agents which can be administered include an antagonist of the ryanodine receptor, such as dantrolene, which has been shown to enhance antisense-mediated exon skipping in patient cells and a mouse model of DMD (G. Kendall et al. Sci Tranl Med 4 164ral60 (2012), incorporated herein by reference).

Methods for the delivery of nucleic acid molecules are described, for example, in Akhtar et al., 1992, Trends Cell Bio., 2:139; and Delivery Strategies for Antisense Oligonucleotide Therapeutics, ed. Akhtar; Sullivan et al., PCT WO 94/02595. These and other protocols can be utilized for the delivery of virtually any nucleic acid molecule, including isolated antisense oligonucleotides or antisense oligonucleotide conjugates described herein.

As detailed below, the pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the antisense oligonucleotides or antisense oligonucleotide conjugates include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

Compositions comprising surface-modified liposomes containing poly (ethylene glycol) lipids (PEG-modified, branched and unbranched or combinations thereof, or long-circulating liposomes or stealth liposomes) can be prepared. Antisense oligonucleotides or antisense oligonucleotide conjugates can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In a further embodiment, antisense oligonucleotide or antisense oligonucleotide conjugate compositions prepared for delivery as described in U.S. Pat. Nos. 6,692,911, 7,163,695 and 7,070,807. In this regard, in one embodiment, an antisense oligonucleotide or antisense oligonucleotide conjugate, or pharmaceutically acceptable salt thereof, is provided in a composition comprising copolymers of lysine and histidine (HK) (as described in U.S. Pat. Nos. 7,163,695, 7,070,807, and 6,692,911) either alone or in combination with PEG (e.g., branched or unbranched PEG or a mixture of both), in combination with PEG and a targeting moiety or any of the foregoing in combination with a crosslinking agent. In certain embodiments, antisense oligonucleotides or antisense oligonucleotide conjugate, or pharmaceutically acceptable salt thereof, are included in compositions comprising gluconic-acid-modified polyhistidine or gluconylated-polyhistidine/transferrin-polylysine. One skilled in the art will also recognize that amino acids with properties similar to His and Lys may be substituted within the composition.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation comprises an excipient selected from cyclodextrins, celluloses, liposomes, micelle forming agents, e.g., bile acids, and polymeric carriers, e.g., polyesters and polyanhydrides; and an antisense oligonucleotide or antisense oligonucleotide conjugate, or pharmaceutically acceptable salt thereof. In certain embodiments, an aforementioned formulation renders orally bioavailable an antisense oligonucleotide or antisense oligonucleotide conjugate, or pharmaceutically acceptable salt thereof.

Methods of preparing these formulations or compositions include the step of bringing into association an antisense oligonucleotide or antisense oligonucleotide conjugate, or pharmaceutically acceptable salt thereof, with the carrier and, optionally, one or more accessory ingredients. In general, the formulations are prepared by uniformly and intimately bringing into association an antisense oligonucleotide or antisense oligonucleotide conjugate, or pharmaceutically acceptable salt thereof, with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an antisense oligonucleotide or antisense oligonucleotide conjugate, or pharmaceutically acceptable salt thereof, as an active ingredient. An antisense oligonucleotide or antisense oligonucleotide conjugate, or pharmaceutically acceptable salt thereof, may also be administered as a bolus, electuary or paste.

In solid dosage forms for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of an antisense oligonucleotide or antisense oligonucleotide conjugate, or pharmaceutically acceptable salt thereof, include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more antisense oligonucleotide or antisense oligonucleotide conjugate, or pharmaceutically acceptable salt thereof, with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations or dosage forms for the topical or transdermal administration of an antisense oligonucleotide or antisense oligonucleotide conjugate, or pharmaceutically acceptable salt thereof, as provided herein include powders, sprays, ointments, pastes, creams, lotions, gels, solutions, patches and inhalants. The antisense oligonucleotide or antisense oligonucleotide conjugate, or pharmaceutically acceptable salt thereof, may be mixed under sterile conditions with a pharmaceutically-acceptable carrier, and with any preservatives, buffers, or propellants which may be required. The ointments, pastes, creams and gels may contain, in addition to an antisense oligonucleotide or antisense oligonucleotide conjugate, or pharmaceutically acceptable salt thereof, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an antisense oligonucleotide or antisense oligonucleotide conjugate, or pharmaceutically acceptable salt thereof, provided herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an antisense oligonucleotide or antisense oligonucleotide conjugate to the body. Such dosage forms can be made by dissolving or dispersing the antisense oligonucleotide or antisense oligonucleotide conjugate, or pharmaceutically acceptable salt thereof, in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise one or more antisense oligonucleotides or antisense oligonucleotide conjugate, or pharmaceutically acceptable salt thereof, in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject oligomers may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsule matrices of the subject oligomers in biodegradable polymers such as polylactide-polyglycolide. Depending on the ratio of oligomer to polymer, and the nature of the particular polymer employed, the rate of antisense oligonucleotide or antisense oligonucleotide conjugate release can be controlled. Examples of other biodegradable polymers include poly(orthoesters) and poly(anhydrides). Depot injectable formulations may also prepared by entrapping the drug in liposomes or microemulsions that are compatible with body tissues.

When the antisense oligonucleotides or antisense oligonucleotide conjugate, or pharmaceutically acceptable salt thereof, are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

As noted above, the formulations or preparations may be given orally, parenterally, systemically, topically, rectally or intramuscular administration. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

Regardless of the route of administration selected, the antisense oligonucleotides or antisense oligonucleotide conjugate, or pharmaceutically acceptable salt thereof, which may be used in a suitable hydrated form, and/or the pharmaceutical compositions, may be formulated into pharmaceutically-acceptable dosage forms by conventional methods known to those of skill in the art. Actual dosage levels of the active ingredients in the pharmaceutical compositions may be varied so as to obtain an amount of the active ingredient which is effective to achieve the desired therapeutic response for a particular patient, composition, and mode of administration, without being unacceptably toxic to the human patient.

The selected dosage level will depend upon a variety of factors including the activity of the particular antisense oligonucleotide or antisense oligonucleotide conjugate, or pharmaceutically acceptable salt thereof, employed, or the ester, salt or amide thereof, the route of administration, the time of administration, the rate of excretion or metabolism of the particular antisense oligonucleotide or antisense oligonucleotide conjugate, or pharmaceutically acceptable salt thereof, being employed, the rate and extent of absorption, the duration of the treatment, other drugs, compounds and/or materials used in combination with the particular antisense oligonucleotide or antisense oligonucleotide conjugate, or pharmaceutically acceptable salt thereof, employed, the age, sex, weight, condition, general health and prior medical history of the human patient being treated, and like factors well known in the medical arts.

A physician or veterinarian having ordinary skill in the art can readily determine and prescribe the effective amount of the pharmaceutical composition required. For example, the physician or veterinarian could start doses of the antisense oligonucleotides or antisense oligonucleotide conjugates, or pharmaceutically acceptable salts thereof, employed in the pharmaceutical composition at levels lower than that required in order to achieve the desired therapeutic effect and gradually increase the dosage until the desired effect is achieved. In general, a suitable daily dose of an antisense oligonucleotide or antisense oligonucleotide conjugate, conjugate, or pharmaceutically acceptable salt thereof, will be that amount of the compound which is the lowest dose effective to produce a therapeutic effect. Such an effective dose will generally depend upon the factors described above.

In some embodiments, a dose of the antisense oligonucleotide (e.g., PMO) or antisense oligonucleotide conjugate (e.g., PPMO), or pharmaceutically salt thereof, is about 0.005 mg/kg to about 300 mg/kg. In some embodiments, a dose is at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg. In some embodiments, a dose is about 0.005 mg/kg to about 200 mg/kg, about 0.1 mg/kg to about 100 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, or about 80 mg/kg to about 300 mg/kg. In some embodiments, a dose is about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg.

In some embodiments, the antisense oligonucleotide (e.g., PMO) or antisense oligonucleotide conjugate (e.g., PPMO), or pharmaceutically salt thereof, is administered, generally at regular intervals (e.g., every week, two weeks, three weeks, four weeks, monthly). For example, the antisense oligonucleotide or antisense oligonucleotide conjugate, or pharmaceutically acceptable salt thereof, may be administered once every week, two weeks, three weeks, four weeks, or monthly by intravenous infusion. In one embodiment, the antisense oligonucleotide or antisense oligonucleotide conjugate, or pharmaceutically acceptable salt thereof, may be administered once every week, two weeks, three weeks, four weeks, or month by intravenous infusion. In some embodiments, the antisense oligonucleotide (e.g., PMO) or antisense oligonucleotide conjugate (e.g., PPMO), or pharmaceutically acceptable salt thereof, is administered once every four weeks.

Administration may be followed by, or concurrent with, administration of an antibiotic, steroid or other therapeutic agent. The treatment regimen may be adjusted (dose, frequency, route, etc.) as indicated, based on the results of immunoassays, other biochemical tests and physiological examination of the human patient under treatment.

Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres, as described herein and known in the art. In certain embodiments, microemulsification technology may be utilized to improve bioavailability of lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other benefits, microemulsification provides enhanced bioavailability by preferentially directing absorption to the lymphatic system instead of the circulatory system, which thereby bypasses the liver, and prevents destruction of the compounds in the hepatobiliary circulation.

The formulations can contain micelles formed from an antisense oligonucleotide or antisense oligonucleotide conjugate, or pharmaceutically acceptable salt thereof, as provided herein and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the antisense oligonucleotide or antisense oligonucleotide conjugate, or pharmaceutically acceptable salt thereof, and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be particularly useful, including Gelucire-series, Labrafil, Labrasol, or Laurogly col (all manufactured and distributed by Gattefosse Corporation, Saint Priest, France). PEG-mono-oleate. PEG-di-oleate, PEG-mono-laurate and di-laurate, Lecithin, Polysorbate 80, etc (produced and distributed by a number of companies in USA and worldwide).

In certain embodiments, the delivery may occur by use of liposomes, nanocapsules, microparticles, microspheres, lipid particles, vesicles, and the like, for the introduction of the antisense oligonucleotide or antisense oligonucleotide conjugate, or pharmaceutically acceptable salt thereof, into suitable host cells. In particular, the antisense oligonucleotide or antisense oligonucleotide conjugate, or pharmaceutically acceptable salt thereof, may be formulated for delivery either encapsulated in a lipid particle, a liposome, a vesicle, a nanosphere, a nanoparticle or the like. The formulation and use of such delivery vehicles can be carried out using known and conventional techniques.

Hydrophilic polymers suitable for use with an antisense oligonucleotide or antisense oligonucleotide conjugate, or pharmaceutically acceptable salt thereof, are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In certain embodiments, polymers have a molecular weight of from about 100 or 120 daltons up to about 5,000 or 10,000 daltons, or from about 300 daltons to about 5,000 daltons. In other embodiments, the polymer is polyethyleneglycol having a molecular weight of from about 100 to about 5,000 daltons, or having a molecular weight of from about 300 to about 5,000 daltons. In certain embodiments, the polymer is polyethyleneglycol of 750 daltons (PEG(750)). Polymers may also be defined by the number of monomers therein; a preferred embodiment utilizes polymers of at least about three monomers, such PEG polymers consisting of three monomers (approximately 150 daltons).

Other hydrophilic polymers which may be suitable for use with an antisense oligonucleotide or antisense oligonucleotide conjugate, or pharmaceutically acceptable salt thereof, include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7 or 8 glucose units, designated by the Greek letter α, β, or γ, respectively. The glucose units are linked by α-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17α-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 pm in diameter; large unilamellar vesicles (LUVS) are typically larger than 0.05 pm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

Formulations comprising liposomes can contain an antisense oligonucleotide or antisense oligonucleotide conjugate, or pharmaceutically acceptable salt thereof, where the liposome membrane is formulated to provide a liposome with increased carrying capacity. Alternatively or in addition, the antisense oligonucleotide or antisense oligonucleotide conjugate, or pharmaceutically acceptable salt thereof, may be contained within, or adsorbed onto, the liposome bilayer of the liposome. An antisense oligonucleotide or antisense oligonucleotide conjugate may be aggregated with a lipid surfactant and carried within the liposome's internal space; in these cases, the liposome membrane is formulated to resist the disruptive effects of the active agent-surfactant aggregate.

According to one embodiment, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes are in solubilized form. Aggregates of surfactant and active agent (such as emulsions or micelles containing the active agent of interest) may be entrapped within the interior space of liposomes. A surfactant acts to disperse and solubilize the active agent, and may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMOs in the micromolar range; higher CMC surfactants may be utilized to prepare micelles entrapped within liposomes.

Liposomes may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993. For example, liposomes may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In certain embodiments, reagents such as DharmaFECT® and Lipofectamine® may be utilized to introduce polynucleotides or proteins into cells.

The release characteristics of a formulation depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

In addition to the methods provided herein, the antisense oligonucleotide or antisense oligonucleotide conjugate, or pharmaceutically acceptable salt thereof, may be formulated for administration in any convenient way for use in human or veterinary medicine, by analogy with other pharmaceuticals. The antisense oligonucleotide or antisense oligonucleotide conjugate, or pharmaceutically acceptable salt thereof, and its corresponding formulation may be administered alone or in combination with other therapeutic strategies in the treatment of muscular dystrophy, such as myoblast transplantation, stem cell therapies, administration of aminoglycoside antibiotics, proteasome inhibitors, and up-regulation therapies (e.g., upregulation of utrophin, an autosomal paralogue of dystrophin).

The routes of administration described are intended only as a guide since a skilled practitioner will be able to determine readily the optimum route of administration and any dosage for any particular animal and condition. Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (e.g., adeno-associated viral vectors) (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813.

### V. KITS

The invention also provides kits for treatment of a human patient with a genetic disease which kit comprises at least an antisense oligonucleotide or antisense oligonucleotide conjugate, or pharmaceutically acceptable salt thereof (e.g., one or more antisense oligonucleotides capable of targeting a particular sequence, such as one or more of exons 1-79 of the dystrophin gene (for example, Exon 51)), packaged in a suitable container, together with instructions for its use. The kits may also contain peripheral reagents such as buffers, stabilizers, etc. Those of ordinary skill in the field should appreciate that applications of the above method has wide application for identifying antisense molecules suitable for use in the treatment of many other diseases.

### VII. EXAMPLES

### Example 1: Exon Skipping in Cynomolgus Monkeys Over 28 Days after Single Intravenous Administration of PPMO #1

A total of 20 naïve, male cynomolgus monkeys were enrolled in the study with 4 males in Groups A, and 8 males in each of Groups B and C. All animals received a 1-hour IV infusion on Day 1. Doses were administered through the saphenous or cephalic vein with an IV catheter. Group A animals were administered Vehicle Control. Groups B and C were administered PPMO #1 (as .6HCl salt) at dose levels of 30 and 60 mg/kg, respectively. PPMO #1 concentrations for Groups B and C were 3 and 6 mg/mL, respectively, and dose volume for all animals was 10 mL/kg, as shown in Table 2.

TABLE 2

| | | | Dose | | Dose | Dose |
|---|---|---|---|---|---|---|
| Group | No. of | Test | Level | Conc. | Volume | Route/ |
| No. | Animals | Material | (mg/kg) | (mg/mL) | (mL/kg) | Regimen |
| A | 4 | Vehicle | 0 | 0 | 10 | 1-Hour IV |
| | | Control | | | | Infusion |
| B | 8 | PPMO #1 | 30 | 3 | | on Day 1 |
| C | 8 | | 60 | 6 | | |

### Results:

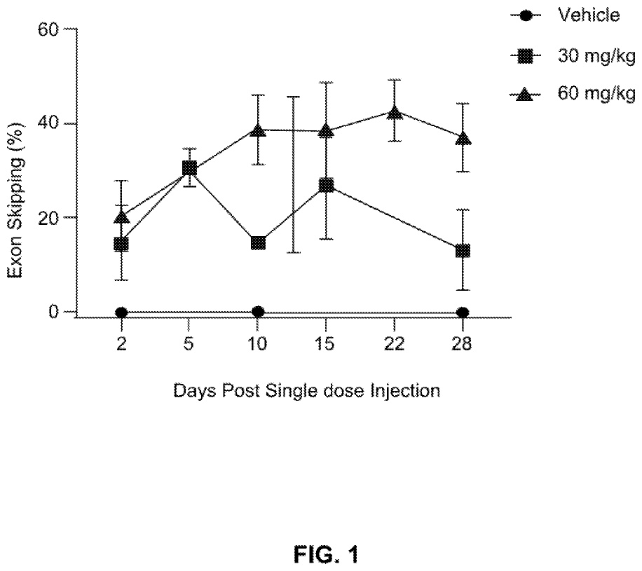

The bicep samples were homogenized and processed for dd-PCR to determine the levels of exon 51 skipping over 28 days. No exon skipping was detected from in biopsy samples from the vehicle treated groups. Exon skipping was detected in the 30 mg/kg and 60 mg/kg treated animals. For both the 30 and 60 mg/kg groups, exon skipping started on Day 2 and continued through Day 28 (end of study). On Day 28, exon skipping levels were 13.3%±8.6 and 37.2%±7.2 at the 30 and 60 mg/kg doses, respectively. The exon skipping data are depicted in FIG. 1.

Exon skipping levels were dose dependent, no exon skipping was measured in the vehicle treated samples, and the highest levels of exon skipping were detected at the 60 mg/kg group. Exon skipping was measured as early as 24h post dose and was detected at all time points until the last time point of the study, i.e. Day 28. Exon skipping levels on Day 28 were 13.3%±8.6 and 37.2%±7.2 at the 30 and 60 mg/kg doses, respectively. Exon skipping may have persisted longer than 28 days post single dose. The persistence of exon skipping for 28 days in the biceps biopsy samples can be used to support the monthly (i.e., once every four weeks) dosing regimen in the clinic. The data demonstrate that 30 or 60 mg/kg doses, administered every four weeks, can lead to reasonably high exon skipping with duration of effect for at least 28 days. The high potency of PPMO #1 along with the long duration of effect may translate into therapeutically effective protein expression of functionally truncated dystrophin in DMD patients.

### Example 2: Assessment of Renal Safety Parameters Based on Clinical Pathology and Histopathological Assessment of Kidney

A total of 28 naïve, male cynomolgus monkeys were enrolled in the study with 4 animals in Group 1, and 12 animals each in Groups 2 and 3. All animals received 1-hour IV infusions on Days 1, 29, 57, and 85. Group 1 animals were administered Vehicle Control. Groups 2 and 3 were administered PPMO #1 (as .6HCl salt) at dose levels of 30 and 60 mg/kg, respectively. PPMO #1 concentrations for Groups 2 and 3 were 3 and 6 mg/mL, respectively, and dose volume for all animals was 10 mL/kg.

All animals were monitored throughout the study with clinical observations and body weight measurements. Blood was collected pre-dose and at 1, 2, 4, 8, 12, 16, and 24 hours after the start of each infusion to provide plasma for PK analysis; urine was collected at 8 and 24 hours post-dose. Blood was collected for hematology and serum chemistry before the start of Day 1 dosing, and at Days 30, 58, and 86. Muscle biopsy and necropsy tissue collection was performed according to the schedule below in Table 3:

TABLE 3
Study Design

| | | | Dose | | Dose | Dose |
|---|---|---|---|---|---|---|
| Group | No. of | Test | Level | Conc. | Volume | Route/ |
| No. | Animals | Material | (mg/kg) | (mg/mL) | (mL/kg) | Regimen |
| 1 | 4 | Vehicle | 0 | 0 | 10 | 1-Hour IV |
| | | Control | | | | Infusion on |
| 2 | 12 | PPMO #1 | 30 | 3 | | Days 1, 29, |
| 3 | 12 | | 60 | 6 | | 57, and 85 |

Administration of PPMO #1 (as .6HCl salt) by intravenous infusion for 4 doses on Days 1, 29, 57, and 85 was clinically well tolerated under the conditions of this study in cynomolgus monkeys at dose levels of 30 and 60 mg/kg/dose. The highest dose in this study (60 mg/kg) corresponded to Cₘₐₓ of 266000±15500 ng/mL and an AUC_{inf} of 415000±254000 h*ng/mL on Day 1, and a Cₘₐₓ of 312000±38100 ng/mL and an AUC_{inf} of 479000±84900 h*ng/mL on Day 85. The main microscopic observation in the kidney was the accumulation of basophilic granules in tubular epithelium of the kidneys which was not considered adverse. Although there were some red blood cell mass decreases in the 30 and 60 mg/kg groups, there were no time or dose-dependent changes in the hematology overall or in the serum chemistry and urinalysis parameters that would indicate significant systemic changes.

### Example 3: Single Ascending Dose Study

Patients will receive a single dose of PPMO #1 (as a .6HCl salt) (either 0.3, 1.0, 2.0, 4.0, or 6.0 mg/kg) administered as an intravenous (IV) infusion.

Selected Inclusion Criteria:
I 2. Has a genetic diagnosis of DMD and an out-of-frame deletion mutation of the DMD gene amenable to exon 51 skipping treatment.
I 4. Has been on a stable dose of oral corticosteroids for at least 12 weeks prior to study drug administration, or has not received corticosteroids for at least 12 weeks prior to study drug administration.

Selected Exclusion Criteria:
A. Has a left ventricular ejection fraction (LVEF)<40% based on an ECHO performed within 3 months.
B. Has a QT interval corrected with Fridericia's formula (QTcF)≥450 msec in an ECG.
C. Has a forced vital capacity (FVC)<40% of predicted value within 3 months of Screening.
D. Platelet count <150×103 μL.

Criteria for Evaluation:

Safety Endpoints: Safety endpoints will include the AEs, clinical laboratory tests, safety biomarkers of renal function, ECGs, ECHOs, physical examinations, and vital signs.

Pharmacokinetic Endpoints: The following PK parameters will be calculated: maximum observed drug concentration (Cₘₐₓ), time to maximum concentration (Tₘₐₓ), area under the concentration-time curve (AUC) from Hour 0 to the last measurable concentration (AUC₀₋ₜ), AUC extrapolated to infinity (AUC_{0-∞}), apparent terminal elimination rate constant (λZ), apparent terminal elimination half-life (t_{1/2}), plasma clearance (CL), volume of distribution at the terminal phase (V_{z}), and volume of distribution at steady state (Vₛₛ).

### Example 4: Multiple Ascending Dose Study

Patients will be assigned to 1 of 4 cohorts: PPMO #1 at 4.0, 10.0, 16.0, or 20.0 mg/kg (as a .6HCl salt).

### Part A Dose Administration and Study Assessments:

PPMO #1 (as .6HCl salt) will be administered IV every 4 weeks (defined as every 28 days [4 days]). Each cohort will complete at least 12 weeks of dosing. Patients will continue to receive drug every 4 weeks at the dose level of their assigned cohort until the maximum tolerated dose (MTD) is identified.

All patients will undergo an additional muscle and skin biopsy (for evaluation of exon-skipping and exploratory exon-skipping, dystrophin protein production, and tissue PPMO levels) at Week 12 of their participation in Part A.

Assessments of pulmonary and physical function and quality of life will be performed every 12 weeks (±4 days) after initial dosing.

The transition from Part A to Part B of the study will occur once the MTD has been determined in Part A.

### Part B—Dose Expansion

Patients will proceed directly from Part A to Part B once the MTD has been determined in Part A. Upon MTD confirmation, the next dose administered to all patients will be the MTD.

### Treatment and Observation Period

Patients entering Part B of the study will receive additional treatment with PPMO #1 (as .6HCl salt), administered IV every 4 weeks at the MTD determined in Part A. Patients will continue dosing with PPMO #1 every 4 weeks at the MTD determined in Part A, for a minimum duration of 24 weeks. The clinical laboratory tests, other safety assessments, and functional and quality of life assessments performed in Part A will also be performed in Part B.

All patients will also undergo an additional muscle and skin biopsy (for evaluation of exon-skipping and exploratory exon-skipping, dystrophin protein production, and tissue PPMO levels) at Week 24 of their participation in Part B.

### Safety Follow-Up Period (Mandatory for all Patients)

All patients will return to the clinic for the End of Study/Early Termination visit up to 4 weeks (+4 days) after their last dose of PPMO #1. This includes both patients who complete the study and those who withdraw early. Vital sign measurements, physical examination findings, clinical laboratory tests, ECG findings, changes in concomitant medications, and AEs will be assessed at this visit. There should be no interruption in the collection of safety and efficacy data at any point in this study.

Selected Inclusion Criteria:
A. Has a genetic diagnosis of DMD and an out-of-frame deletion mutation of the DMD gene amenable to exon 51-skipping treatment.
B. Has been on a stable dose of oral corticosteroids for at least 12 weeks prior to study drug administration, or has not received corticosteroids for at least 12 weeks prior to study drug administration.

Selected Exclusion Criteria:
A. Has a LVEF<40.0% based on an ECHO performed within 12 weeks.
B. Has a QT interval corrected with Fridericia's formula ≥450 msec in an ECG.
C. Has a FVC<40.0% of predicted value (according to the American Thoracic Society/European Respiratory Society criteria) within 12 weeks.

Dose/Route/Regimen (Test Article):

In Part A (MAD, for dose determination), patients will receive ascending doses of PPMO #1 every 4 weeks, starting at the dose level for their assigned cohort (4.0, 10.0, 16.0, or 20.0 mg/kg), administered by IV infusion over a period of 60 minutes (+5 minutes).

In Part B (dose expansion), patients will receive doses of PPMO #1 every 4 weeks at the MTD determined in Part A, administered by IV infusion over a period of 60 minutes (±5 minutes).

All publications and patent applications cited in this specification are herein incorporated by reference as if each individual publication or patent application were specifically and individually indicated to be incorporated by reference.

1. A method of treating a human patient having Duchenne muscular dystrophy, the method comprising:
administering to the human patient once every four weeks a therapeutically effective amount of an antisense oligonucleotide conjugate, or a pharmaceutically acceptable salt thereof, said the antisense oligonucleotide conjugate comprising a cell penetrating peptide covalently attached to an oligonucleotide;
wherein said antisense oligonucleotide conjugate, or pharmaceutically acceptable salt thereof, induces exon skipping in the human dystrophin gene.

2. The method of claim 1, wherein the antisense oligonucleotide conjugate, or pharmaceutically acceptable salt thereof, comprises a cell penetrating peptide that is an arginine-rich peptide.

3. The method of claim 1 or 2, wherein the antisense oligonucleotide conjugate, or pharmaceutically acceptable salt thereof, comprises a cell penetrating peptide that is an arginine-rich peptide that is that is -GLY-R₅—R^{a} (SEQ ID NO: 59), —R₅—R^{a} (SEQ ID NO: 60), -GLY-R₆—R^{a} (SEQ ID NO: 57) or —R₆—R^{a} (SEQ ID NO: 58), wherein R is arginine and R^{a} is hydrogen or an acyl group.

4. The method of any one of claims 1-3, wherein the antisense oligonucleotide conjugate, or pharmaceutically acceptable salt thereof, induces skipping of exon 44, exon 45, exon 50, exon 51, exon 52, or exon 53 target region of the dystrophin pre-mRNA.

5. The method of any one of claims 1-4, wherein the antisense oligonucleotide conjugate is according to Formula (I): 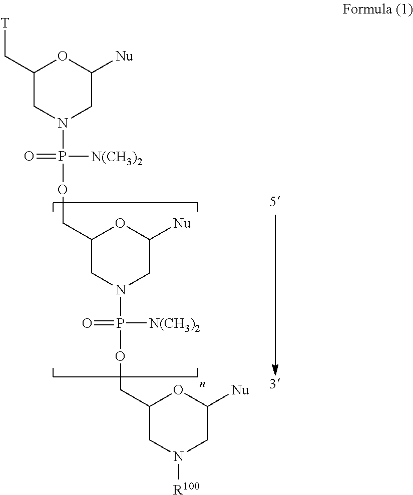
or a pharmaceutically acceptable salt thereof, wherein:
each Nu is a nucleobase which taken together form a targeting sequence;
T′ in Formula (I) is a moiety selected from: 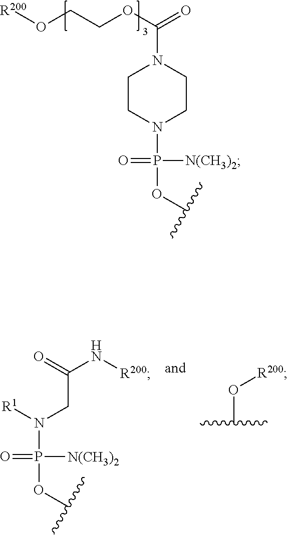
R¹⁰⁰ is a cell-penetrating peptide;
R²⁰⁰ is hydrogen;
R¹ is C₁-C₆ alkyl; and
each Nu from 1 to (n+1) and 5′ to 3′ corresponds to the nucleobases in one of the following:

| Base Sequence [5′ to 3′] | SEQ ID NO: |
|---|---|
| CTC CAA CAT CAA GGA AGA TGG CAT TTC TAG | 1 |
| ACC TCC AAC ATC AAG GAA GAT GGC | 2 |
| GTA CCT CCA ACA TCA AGG AAG ATG GCA TTT | 3 |
| GTA CCT CCA ACA TCA AGG AAG ATG GCA T | 4 |
| TCA AGG AAG ATG GCA TTT CT | 5 |
| UCA AGG AmAGm AmUGm GmCA UUU CU | 6 |
| GTT GCC TCC GGT TCT GAA GGT GTT C | 7 |
| GTT G5mC5mC T5mC5mC GGT T5mC T GAA GGT GTT 5mC | 8 |
| CCT CCG GTT CTG AAG GTG TTC | 9 |
| CTG AAG GTG TTC TTG TAC TTC ATC C | 10 |
| CCT CCG GTT CTG AAG GTG TTC TTG T | 11 |
| GTT GCC TCC GGT TCT GAA GGT GTT CTT G | 12 |
| TTG CCT CCG GTT CTG AAG GTG TTC TTG TAC | 13 |
| CTG TTG CCT CCG GTT CTG AAG GTG | 14 |
| CAT TCA ACT GTT GCC TCC GGT TCT GAA GGT G | 15 |
| CTG TTG CCT CCG GTT CTG | 16 |
| CAA TGC CAT CCT GGA GTT CCT G | 17 |
| GCT GCC CAA TGC CAT CCT GGA GTT CCT GTA AGA T | 18 |
| GCT GCC CAA TGC CAT CCT GGA GTT CCT G | 19 |
| GCT GCC CAA TGC CAT CCT GGA GTT CCT GTA A | 20 |
| CAA TGC CAT CCT GGA GTT CCT GTA AGA TAC C | 21 |
| CAA TGC CAT CCT GGA GTT CCT GTA AGA T | 22 |
| TGC CAT CCT GGA GTT CCT GTA AGA TAC C | 23 |
| GCT GCC CAA TGC CAT CCT GGA GTT CCT GTA AGA | 24 |
| TAC CAA | |
| CAA TGC CAT CCT GGA GTT CCT GTA AGA | 25 |
| GCT GCC CAA TGC CAT CCT GGA GTT CCT GTA AG | 26 |
| GCC CAA TGC CAT CCT GGA GTT CCT GTA AGA TAC C | 27 |
| GCC CAA TGC CAT CCT GGA GTT CCT GTA AGA T | 28 |
| TTG CCG CTG CCC AAT GCC ATC CTG GAG TTC CTG TAA | 29 |
| GAT | |
| GCC CAA TGC CAT CCT GGA GTT CCT GTA A | 30 |
| GCC GCT GCC CAA TGC CAT CCT GGA GTT CCT GTA A | 31 |
| GCC CAA TGC CAT CCT GGA GTT CCT G | 32 |
| GCC GCT GCC CAA TGC CAT CCT GGA GTT CCT G | 33 |
| m5C-G-m5C-T-G-C-m5C-m5C-A-A-T-G-m5C-m5C-A-U-m5C- | 34 |
| m5C | |
| GAT CTG TCA AAT CGC CTG CAG GTA A | 35 |
| GAT CTG TCA AAT CGC CTG CAG G | 36 |
| CAG ATC TGT CAA ATC GCC TGC AGG | 37 |
| GAT CTG TCA AAT CGC CTG CAG GT | 38 |
| GAT CTG TCA AAT CGC CTG CAG | 39 |
| CAG ATC TGT CAA ATC GCC TGC AGG T | 40 |
| CAG ATC TGT CAA ATC GCC TGC AG | 41 |
| GGG ATC CAG TAT ACT TAC AGG C | 42 |
| GGG ATC CAG TAT ACT TAC AGG CTC C | 43 |
| GAT CCA GTA TAC TTA CAG GCT CC | 44 |
| GGA TCC AGT ATA CTT ACA GGC TCC | 45 |
| ACT TCC TCT TTA ACA GAA AAG CAT AC | 46 |
| ATC CAG TAT ACT TAC AGG CTC C | 47 |
| GAG CTC AGA TCT TCT AAC TTC CTC T | 48 |
| GGG ATC CAG TAT ACT TAC AGG CTC | 49 |
| ATG GGA TCC AGT ATA CTT ACA GGC TCC | 50 |
| CTG TTC CAA ATC CTG CAT TGT TGC C | 51 |

wherein each T of each of SEQ ID NOS: 1-51 is thymine or uracil.

6. The method of claim 5, wherein each Nu from 1 to (n+1) and 5′ to 3′ of the antisense oligonucleotide conjugate, or pharmaceutically acceptable salt thereof, corresponds to SEQ ID NO: 1, SEQ ID NO: 7, or SEQ ID NO: 17.

7. The method of claim 1, wherein the antisense oligonucleotide conjugate is PPPMO #1, PPMO #2, or PPMO #3, or a pharmaceutically acceptable salt thereof.

8. The method of claim 1, wherein the method comprises administering to the human patient a therapeutically effective amount of an antisense oligonucleotide conjugate according to Formula (II): 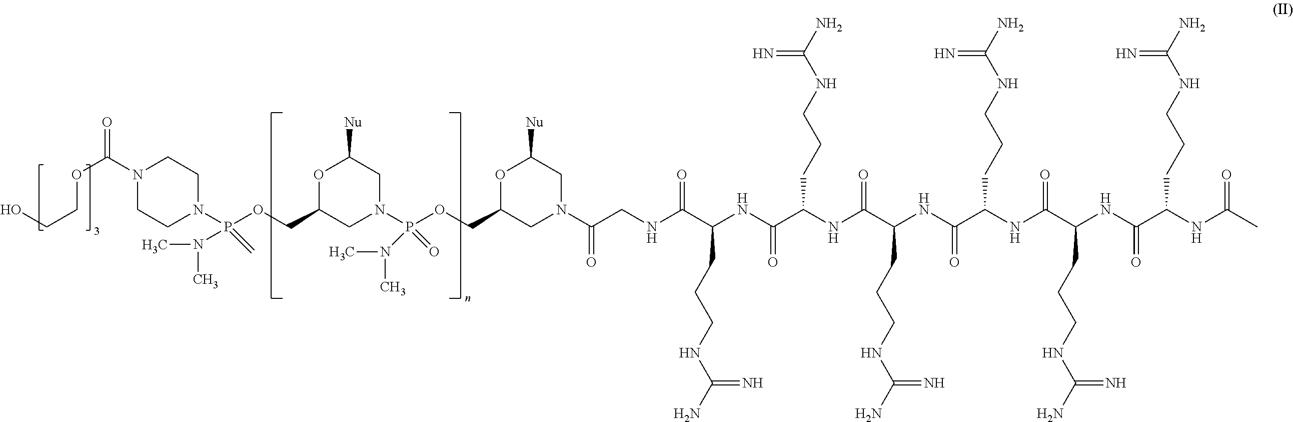
or a pharmaceutically acceptable salt thereof, each Nu from 1 to (n+1) and 5′ to 3′ corresponds to the nucleobases in one of the following:

| Base Sequence [5′ to 3′] | SEQ ID NO: |
|---|---|
| CTC CAA CAT CAA GGA AGA TGG CAT TTC TAG | 1 |
| ACC TCC AAC ATC AAG GAA GAT GGC | 2 |
| GTA CCT CCA ACA TCA AGG AAG ATG GCA TTT | 3 |
| GTA CCT CCA ACA TCA AGG AAG ATG GCA T | 4 |
| TCA AGG AAG ATG GCA TTT CT | 5 |
| UCA AGG AmAGm AmUGm GmCA UUU CU | 6 |
| GTT GCC TCC GGT TCT GAA GGT GTT C | 7 |
| GTT G5mC5mC T5mC5mC GGT T5mC T GAA GGT GTT 5mC | 8 |
| CCT CCG GTT CTG AAG GTG TTC | 9 |
| CTG AAG GTG TTC TTG TAC TTC ATC C | 10 |
| CCT CCG GTT CTG AAG GTG TTC TTG T | 11 |
| GTT GCC TCC GGT TCT GAA GGT GTT CTT G | 12 |
| TTG CCT CCG GTT CTG AAG GTG TTC TTG TAC | 13 |
| CTG TTG CCT CCG GTT CTG AAG GTG | 14 |
| CAT TCA ACT GTT GCC TCC GGT TCT GAA GGT G | 15 |
| CTG TTG CCT CCG GTT CTG | 16 |
| CAA TGC CAT CCT GGA GTT CCT G | 17 |
| GCT GCC CAA TGC CAT CCT GGA GTT CCT GTA AGA T | 18 |
| GCT GCC CAA TGC CAT CCT GGA GTT CCT G | 19 |
| GCT GCC CAA TGC CAT CCT GGA GTT CCT GTA A | 20 |
| CAA TGC CAT CCT GGA GTT CCT GTA AGA TAC C | 21 |
| CAA TGC CAT CCT GGA GTT CCT GTA AGA T | 22 |
| TGC CAT CCT GGA GTT CCT GTA AGA TAC C | 23 |
| GCT GCC CAA TGC CAT CCT GGA GTT CCT GTA AGA | 24 |
| TAC CAA | |
| CAA TGC CAT CCT GGA GTT CCT GTA AGA | 25 |
| GCT GCC CAA TGC CAT CCT GGA GTT CCT GTA AG | 26 |
| GCC CAA TGC CAT CCT GGA GTT CCT GTA AGA TAC C | 27 |
| GCC CAA TGC CAT CCT GGA GTT CCT GTA AGA T | 28 |
| TTG CCG CTG CCC AAT GCC ATC CTG GAG TTC CTG TAA | 29 |
| GAT | |
| GCC CAA TGC CAT CCT GGA GTT CCT GTA A | 30 |
| GCC GCT GCC CAA TGC CAT CCT GGA GTT CCT GTA A | 31 |
| GCC CAA TGC CAT CCT GGA GTT CCT G | 32 |
| GCC GCT GCC CAA TGC CAT CCT GGA GTT CCT G | 33 |
| m5C-G-m5C-T-G-C-m5C-m5C-A-A-T-G-m5C-m5C-A-U-m5C- | 34 |
| m5C | |
| GAT CTG TCA AAT CGC CTG CAG GTA A | 35 |
| GAT CTG TCA AAT CGC CTG CAG G | 36 |
| CAG ATC TGT CAA ATC GCC TGC AGG | 37 |
| GAT CTG TCA AAT CGC CTG CAG GT | 38 |
| GAT CTG TCA AAT CGC CTG CAG | 39 |
| CAG ATC TGT CAA ATC GCC TGC AGG T | 40 |
| CAG ATC TGT CAA ATC GCC TGC AG | 41 |
| GGG ATC CAG TAT ACT TAC AGG C | 42 |
| GGG ATC CAG TAT ACT TAC AGG CTC C | 43 |
| GAT CCA GTA TAC TTA CAG GCT CC | 44 |
| GGA TCC AGT ATA CTT ACA GGC TCC | 45 |
| ACT TCC TCT TTA ACA GAA AAG CAT AC | 46 |
| ATC CAG TAT ACT TAC AGG CTC C | 47 |
| GAG CTC AGA TCT TCT AAC TTC CTC T | 48 |
| GGG ATC CAG TAT ACT TAC AGG CTC | 49 |
| ATG GGA TCC AGT ATA CTT ACA GGC TCC | 50 |
| CTG TTC CAA ATC CTG CAT TGT TGC C | 51 |

wherein A is 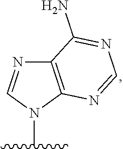 [SMILES: *n1cnc2c(N)ncnc21]
C is 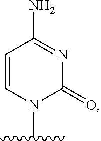 [SMILES: *n1ccc(N)nc1=O]
G is 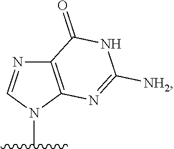 [SMILES: *n1cnc2c(=O)[nH]c(N)nc21]
T is 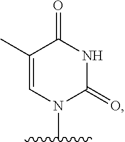 [SMILES: *n1cc(C)c(=O)[nH]c1=O]
U is 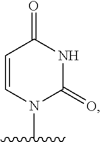 [SMILES: *n1ccc(=O)[nH]c1=O]
Gm is methylated guanine, Am is methylated adenine, and m5C is

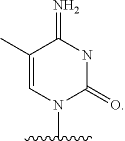 [SMILES: *n1cc(C)c(=N)[nH]c1=O]

9. The method of claim 8, wherein each Nu from 1 to (n+1) and 5′ to 3′ of the antisense oligonucleotide conjugate, or pharmaceutically acceptable salt thereof, of Formula (II) corresponds to SEQ ID NO: 1, SEQ ID NO: 7, or SEQ ID NO: 17.

10. The method of any one of claims 1-9, wherein the antisense oligonucleotide conjugate is in free base form.

11. The method of any one of claims 1-9, wherein the antisense oligonucleotide conjugate is a pharmaceutically acceptable salt.

12. The method of claim 11, wherein the antisense oligonucleotide conjugate is a halide salt.

13. The method of any one of claims 11-12, wherein the antisense oligonucleotide conjugate is an HCl salt.

14. The method of claim 13, wherein the HCl salt of the antisense oligonucleotide conjugate is a 6HCl salt.

15. The method of any one of claims 1-14, wherein the therapeutically effective amount of the antisense oligonucleotide conjugate or pharmaceutically acceptable salt thereof is provided in a pharmaceutical composition formed by dissolving 0.005 mg/kg to about 300 mg/kg of the antisense oligonucleotide conjugate, or pharmaceutically acceptable salt thereof, in an aqueous carrier solution.

16. The method of claim 15, wherein the therapeutically effective amount of the antisense oligonucleotide conjugate is at least 0.05 mg/kg, 0.3 mg/kg, 1 mg/kg, 2 mg/kg, 4 mg/kg, 6 mg/kg, 10 mg/kg, 16 mg/kg, 20 mg/kg, 30 mg/kg, 50 mg/kg, 60 mg/kg, 80 mg/kg, 100 mg/kg, 125 mg/kg, 150 mg/kg, 175 mg/kg, 200 mg/kg, 225 mg/kg, 250 mg/kg, or 275 mg/kg, about 0.005 mg/kg to about 200 mg/kg, about 0.1 mg/kg to about 100 mg/kg, about 0.1 mg/kg to about 80 mg/kg, about 0.1 mg/kg to about 50 mg/kg, about 0.1 mg/kg to about 25 mg/kg, about 20 mg/kg to about 80 mg/kg, about 50 mg/kg to about 100 mg/kg, about 50 mg/kg to about 80 mg/kg, about 80 mg/kg to about 300 mg/kg, about 0.05 mg/kg, about 0.3 mg/kg, about 1 mg/kg, about 2 mg/kg, about 4 mg/kg, about 6 mg/kg, about 10 mg/kg, about 16 mg/kg, about 20 mg/kg, about 30 mg/kg, about 50 mg/kg, about 60 mg/kg, about 80 mg/kg, about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg.

17. The method of any one of claims 1-16, wherein the antisense oligonucleotide conjugate, or pharmaceutically acceptable salt thereof, is administered intravenously.


SEQUENCE LISTING (61 sequences)

SEQ ID NO: 1 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(2): /replace=“u”
  Variation (9)..(9): /replace=“u”
  Variation (19)..(19): /replace=“u”
  Variation (24)..(26): /replace=“u”
  Variation (28)..(28): /replace=“u”
  misc_feature (1)..(30): /note=“Variant nucleotides given in the sequence have no preference with respect to those in the annotations for variant positions”
ctccaacatcaaggaagatggcatttctag

SEQ ID NO: 2 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
  Variation (4)..(4): /replace=“u”
  Variation (11)..(11): /replace=“u”
  Variation (21)..(21): /replace=“u”
  misc_feature (1)..(24): /note=“Variant nucleotides given in the sequence have no preference with respect to those in the annotations for variant positions”
acctccaacatcaaggaagatggc

SEQ ID NO: 3 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(2): /replace=“u”
  Variation (6)..(6): /replace=“u”
  Variation (13)..(13): /replace=“u”
  Variation (23)..(23): /replace=“u”
  Variation (28)..(30): /replace=“u”
  misc_feature (1)..(30): /note=“Variant nucleotides given in the sequence have no preference with respect to those in the annotations for variant positions”
gtacctccaacatcaaggaagatggcattt

SEQ ID NO: 4 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(2): /replace=“u”
  Variation (6)..(6): /replace=“u”
  Variation (13)..(13): /replace=“u”
  Variation (23)..(23): /replace=“u”
  Variation (28)..(28): /replace=“u”
  misc_feature (1)..(28): /note=“Variant nucleotides given in the sequence have no preference with respect to those in the annotations for variant positions”
gtacctccaacatcaaggaagatggcat

SEQ ID NO: 5 - DNA, 20 bp, Artificial Sequence
  source: /note=“Description of Artificial Sequence Synthetic oligonucleotide”
  source: /note=“Description of Combined DNA/RNA Molecule Synthetic oligonucleotide”
  Variation (1)..(1): /replace=“u”
  Variation (11)..(11): /replace=“u”
  Variation (16)..(18): /replace=“u”
  Variation (20)..(20): /replace=“u”
  misc_feature (1)..(20): /note=“Variant nucleotides given in the sequence have no preference with respect to those in the annotations for variant positions”
tcaaggaagatggcatttct

SEQ ID NO: 6 - RNA, 20 nt, Artificial Sequence
  source: /note=“Description of Artificial Sequence Synthetic oligonucleotide”
ucaaggaagauggcauuucu

SEQ ID NO: 7 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(3): /replace=“u”
  Variation (7)..(7): /replace=“u”
  Variation (12)..(13): /replace=“u”
  Variation (15)..(15): /replace=“u”
  Variation (21)..(21): /replace=“u”
  Variation (23)..(24): /replace=“u”
  misc_feature (1)..(25): /note=“Variant nucleotides given in the sequence have no preference with respect to those in the annotations for variant positions”
gttgcctccggttctgaaggtgttc

SEQ ID NO: 8 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(3): /replace=“u”
  Variation (7)..(7): /replace=“u”
  Variation (12)..(13): /replace=“u”
  Variation (15)..(15): /replace=“u”
  Variation (21)..(21): /replace=“u”
  Variation (23)..(24): /replace=“u”
  misc_feature (1)..(25): /note=“Variant nucleotides given in the sequence have no preference with respect to those in the annotations for variant positions”
gttgcctccggttctgaaggtgttc

SEQ ID NO: 9 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (8)..(9): /replace=“u”
  Variation (11)..(11): /replace=“u”
  Variation (17)..(17): /replace=“u”
  Variation (19)..(20): /replace=“u”
  misc_feature (1)..(21): /note=“Variant nucleotides given in the sequence have no preference with respect to those in the annotations for variant positions”
cctccggttctgaaggtgttc

SEQ ID NO: 10 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(2): /replace=“u”
  Variation (8)..(8): /replace=“u”
  Variation (10)..(11): /replace=“u”
  Variation (13)..(14): /replace=“u”
  Variation (16)..(16): /replace=“u”
  Variation (19)..(20): /replace=“u”
  Variation (23)..(23): /replace=“u”
  misc_feature (1)..(25): /note=“Variant nucleotides given in the sequence have no preference with respect to those in the annotations for variant positions”
ctgaaggtgttcttgtacttcatcc

SEQ ID NO: 11 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (8)..(9): /replace=“u”
  Variation (11)..(11): /replace=“u”
  Variation (17)..(17): /replace=“u”
  Variation (19)..(20): /replace=“u”
  Variation (22)..(23): /replace=“u”
  Variation (25)..(25): /replace=“u”
  misc_feature (1)..(25): /note=“Variant nucleotides given in the sequence have no preference with respect to those in the annotations for variant positions”
cctccggttctgaaggtgttcttgt

SEQ ID NO: 12 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(3): /replace=“u”
  Variation (7)..(7): /replace=“u”
  Variation (12)..(13): /replace=“u”
  Variation (15)..(15): /replace=“u”
  Variation (21)..(21): /replace=“u”
  Variation (23)..(24): /replace=“u”
  Variation (26)..(27): /replace=“u”
  misc_feature (1)..(28): /note=“Variant nucleotides given in the sequence have no preference with respect to those in the annotations for variant positions”
gttgcctccggttctgaaggtgttcttg

SEQ ID NO: 13 - DNA, 30 bp, Artificial Sequence
  source: /note=“Description of Artificial Sequence Synthetic oligonucleotide”
  source: /note=“Description of Combined DNA/RNA Molecule Synthetic oligonucleotide”
  Variation (1)..(2): /replace=“u”
  Variation (6)..(6): /replace=“u”
  Variation (11)..(12): /replace=“u”
  Variation (14)..(14): /replace=“u”
  Variation (20)..(20): /replace=“u”
  Variation (22)..(23): /replace=“u”
  Variation (25)..(26): /replace=“u”
  Variation (28)..(28): /replace=“u”
  misc_feature (1)..(30): /note=“Variant nucleotides given in the sequence have no preference with respect to those in the annotations for variant positions”
ttgcctccggttctgaaggtgttcttgtac

SEQ ID NO: 14 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(2): /replace=“u”
  Variation (4)..(5): /replace=“u”
  Variation (9)..(9): /replace=“u”
  Variation (14)..(15): /replace=“u”
  Variation (17)..(17): /replace=“u”
  Variation (23)..(23): /replace=“u”
  misc_feature (1)..(24): /note=“Variant nucleotides given in the sequence have no preference with respect to those in the annotations for variant positions”
ctgttgcctccggttctgaaggtg

SEQ ID NO: 15 - DNA, 31 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(4): /replace=“u”
  Variation (9)..(9): /replace=“u”
  Variation (11)..(12): /replace=“u”
  Variation (16)..(16): /replace=“u”
  Variation (21)..(22): /replace=“u”
  Variation (24)..(24): /replace=“u”
  Variation (30)..(30): /replace=“u”
  misc_feature (1)..(31): /note=“Variant nucleotides given in the sequence have no preference with respect to those in the annotations for variant positions”
cattcaactgttgcctccggttctgaaggtg

SEQ ID NO: 16 - DNA, 18 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(2): /replace=“u”
  Variation (4)..(5): /replace=“u”
  Variation (9)..(9): /replace=“u”
  Variation (14)..(15): /replace=“u”
  Variation (17)..(17): /replace=“u”
  misc_feature (1)..(18): /note=“Variant nucleotides given in the sequence have no preference with respect to those in the annotations for variant positions”
ctgttgcctccggttctg

SEQ ID NO: 17 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
  Variation (4)..(4): /replace=“u”
  Variation (9)..(9): /replace=“u”
  Variation (12)..(12): /replace=“u”
  Variation (17)..(18): /replace=“u”
  Variation (21)..(21): /replace=“u”
  misc_feature (1)..(22): /note=“Variant nucleotides given in the sequence have no preference with respect to those in the annotations for variant positions”
caatgccatcctggagttcctg

SEQ ID NO: 18 - DNA, 34 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (10)..(10): /replace=“u”
  Variation (15)..(15): /replace=“u”
  Variation (18)..(18): /replace=“u”
  Variation (23)..(24): /replace=“u”
  Variation (27)..(27): /replace=“u”
  Variation (29)..(29): /replace=“u”
  Variation (34)..(34): /replace=“u”
  misc_feature (1)..(34): /note=“Variant nucleotides given in the sequence have no preference with respect to those in the annotations for variant positions”
gctgcccaatgccatcctggagttcctgtaagat

SEQ ID NO: 19 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (10)..(10): /replace=“u”
  Variation (15)..(15): /replace=“u”
  Variation (18)..(18): /replace=“u”
  Variation (23)..(24): /replace=“u”
  Variation (27)..(27): /replace=“u”
  misc_feature (1)..(28): /note=“Variant nucleotides given in the sequence have no preference with respect to those in the annotations for variant positions”
gctgcccaatgccatcctggagttcctg

SEQ ID NO: 20 - DNA, 31 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (10)..(10): /replace=“u”
  Variation (15)..(15): /replace=“u”
  Variation (18)..(18): /replace=“u”
  Variation (23)..(24): /replace=“u”
  Variation (27)..(27): /replace=“u”
  Variation (29)..(29): /replace=“u”
  misc_feature (1)..(31): /note=“Variant nucleotides given in the sequence have no preference with respect to those in the annotations for variant positions”
gctgcccaatgccatcctggagttcctgtaa

SEQ ID NO: 21 - DNA, 31 bp, Artificial Sequence
  source: /note=“Description of Artificial Sequence Synthetic oligonucleotide”
  source: /note=“Description of Combined DNA/RNA Molecule Synthetic oligonucleotide”
  Variation (4)..(4): /replace=“u”
  Variation (9)..(9): /replace=“u”
  Variation (12)..(12): /replace=“u”
  Variation (17)..(18): /replace=“u”
  Variation (21)..(21): /replace=“u”
  Variation (23)..(23): /replace=“u”
  Variation (28)..(28): /replace=“u”
  misc_feature (1)..(31): /note=“Variant nucleotides given in the sequence have no preference with respect to those in the annotations for variant positions”
caatgccatcctggagttcctgtaagatacc

SEQ ID NO: 22 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
  Variation (4)..(4): /replace=“u”
  Variation (9)..(9): /replace=“u”
  Variation (12)..(12): /replace=“u”
  Variation (17)..(18): /replace=“u”
  Variation (21)..(21): /replace=“u”
  Variation (23)..(23): /replace=“u”
  Variation (28)..(28): /replace=“u”
  misc_feature (1)..(28): /note=“Variant nucleotides given in the sequence have no preference with respect to those in the annotations for variant positions”
caatgccatcctggagttcctgtaagat

SEQ ID NO: 23 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
  Variation (1)..(1): /replace=“u”
  Variation (6)..(6): /replace=“u”
  Variation (9)..(9): /replace=“u”
  Variation (14)..(15): /replace=“u”
  Variation (18)..(18): /replace=“u”
  Variation (20)..(20): /replace=“u”
  Variation (25)..(25): /replace=“u”
  misc_feature (1)..(28): /note=“Variant nucleotides given in the sequence have no preference with respect to those in the annotations for variant positions”
tgccatcctggagttcctgtaagatacc

SEQ ID NO: 24 - DNA, 39 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (10)..(10): /replace=“u”
  Variation (15)..(15): /replace=“u”
  Variation (18)..(18): /replace=“u”
  Variation (23)..(24): /replace=“u”
  Variation (27)..(27): /replace=“u”
  Variation (29)..(29): /replace=“u”
  Variation (34)..(34): /replace=“u”
  misc_feature (1)..(39): /note=“Variant nucleotides given in the sequence have no preference with respect to those in the annotations for variant positions”
gctgcccaatgccatcctggagttcctgtaagataccaa

SEQ ID NO: 25 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
  Variation (4)..(4): /replace=“u”
  Variation (9)..(9): /replace=“u”
  Variation (12)..(12): /replace=“u”
  Variation (17)..(18): /replace=“u”
  Variation (21)..(21): /replace=“u”
  Variation (23)..(23): /replace=“u”
  misc_feature (1)..(27): /note=“Variant nucleotides given in the sequence have no preference with respect to those in the annotations for variant positions”
caatgccatcctggagttcctgtaaga

SEQ ID NO: 26 - DNA, 32 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (10)..(10): /replace=“u”
  Variation (15)..(15): /replace=“u”
  Variation (18)..(18): /replace=“u”
  Variation (23)..(24): /replace=“u”
  Variation (27)..(27): /replace=“u”
  Variation (29)..(29): /replace=“u”
  misc_feature (1)..(32): /note=“Variant nucleotides given in the sequence have no preference with respect to those in the annotations for variant positions”
gctgcccaatgccatcctggagttcctgtaag

SEQ ID NO: 27 - DNA, 34 bp, Artificial Sequence
  source: /note=“Description of Artificial Sequence Synthetic oligonucleotide”
  source: /note=“Description of Combined DNA/RNA Molecule Synthetic oligonucleotide”
  Variation (7)..(7): /replace=“u”
  Variation (12)..(12): /replace=“u”
  Variation (15)..(15): /replace=“u”
  Variation (20)..(21): /replace=“u”
  Variation (24)..(24): /replace=“u”
  Variation (26)..(26): /replace=“u”
  Variation (31)..(31): /replace=“u”
  misc_feature (1)..(34): /note=“Variant nucleotides given in the sequence have no preference with respect to those in the annotations for variant positions”
gcccaatgccatcctggagttcctgtaagatacc

SEQ ID NO: 28 - DNA, 31 bp, Artificial Sequence
  source: /note=“Description of Artificial Sequence Synthetic oligonucleotide”
  source: /note=“Description of Combined DNA/RNA Molecule Synthetic oligonucleotide”
  Variation (7)..(7): /replace=“u”
  Variation (12)..(12): /replace=“u”
  Variation (15)..(15): /replace=“u”
  Variation (20)..(21): /replace=“u”
  Variation (24)..(24): /replace=“u”
  Variation (26)..(26): /replace=“u”
  Variation (31)..(31): /replace=“u”
  misc_feature (1)..(31): /note=“Variant nucleotides given in the sequence have no preference with respect to those in the annotations for variant positions”
gcccaatgccatcctggagttcctgtaagat

SEQ ID NO: 29 - DNA, 39 bp, Artificial Sequence
  source: /note=“Description of Artificial Sequence Synthetic oligonucleotide”
  source: /note=“Description of Combined DNA/RNA Molecule Synthetic oligonucleotide”
  Variation (1)..(2): /replace=“u”
  Variation (8)..(8): /replace=“u”
  Variation (15)..(15): /replace=“u”
  Variation (20)..(20): /replace=“u”
  Variation (23)..(23): /replace=“u”
  Variation (28)..(29): /replace=“u”
  Variation (32)..(32): /replace=“u”
  Variation (34)..(34): /replace=“u”
  Variation (39)..(39): /replace=“u”
  misc_feature (1)..(39): /note=“Variant nucleotides given in the sequence have no preference with respect to those in the annotations for variant positions”
ttgccgctgcccaatgccatcctggagttcctgtaagat

SEQ ID NO: 30 - DNA, 28 bp, Artificial Sequence
  source: /note=“Description of Artificial Sequence Synthetic oligonucleotide”
  source: /note=“Description of Combined DNA/RNA Molecule Synthetic oligonucleotide”
  Variation (7)..(7): /replace=“u”
  Variation (12)..(12): /replace=“u”
  Variation (15)..(15): /replace=“u”
  Variation (20)..(21): /replace=“u”
  Variation (24)..(24): /replace=“u”
  Variation (26)..(26): /replace=“u”
  misc_feature (1)..(28): /note=“Variant nucleotides given in the sequence have no preference with respect to those in the annotations for variant positions”
gcccaatgccatcctggagttcctgtaa

SEQ ID NO: 31 - DNA, 34 bp, Artificial Sequence
  source: /note=“Description of Artificial Sequence Synthetic oligonucleotide”
  source: /note=“Description of Combined DNA/RNA Molecule Synthetic oligonucleotide”
  Variation (6)..(6): /replace=“u”
  Variation (13)..(13): /replace=“u”
  Variation (18)..(18): /replace=“u”
  Variation (21)..(21): /replace=“u”
  Variation (26)..(27): /replace=“u”
  Variation (30)..(30): /replace=“u”
  Variation (32)..(32): /replace=“u”
  misc_feature (1)..(34): /note=“Variant nucleotides given in the sequence have no preference with respect to those in the annotations for variant positions”
gccgctgcccaatgccatcctggagttcctgtaa

SEQ ID NO: 32 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
  Variation (7)..(7): /replace=“u”
  Variation (12)..(12): /replace=“u”
  Variation (15)..(15): /replace=“u”
  Variation (20)..(21): /replace=“u”
  Variation (24)..(24): /replace=“u”
  misc_feature (1)..(25): /note=“Variant nucleotides given in the sequence have no preference with respect to those in the annotations for variant positions”
gcccaatgccatcctggagttcctg

SEQ ID NO: 33 - DNA, 31 bp, Artificial Sequence
  source: /note=“Description of Artificial Sequence Synthetic oligonucleotide”
  source: /note=“Description of Combined DNA/RNA Molecule Synthetic oligonucleotide”
  Variation (6)..(6): /replace=“u”
  Variation (13)..(13): /replace=“u”
  Variation (18)..(18): /replace=“u”
  Variation (21)..(21): /replace=“u”
  Variation (26)..(27): /replace=“u”
  Variation (30)..(30): /replace=“u”
  misc_feature (1)..(31): /note=“Variant nucleotides given in the sequence have no preference with respect to those in the annotations for variant positions”
gccgctgcccaatgccatcctggagttcctg

SEQ ID NO: 34 - DNA, 18 bp, Artificial Sequence
  source: /note=“Description of Artificial Sequence Synthetic oligonucleotide”
  source: /note=“Description of Combined DNA/RNA Molecule Synthetic oligonucleotide”
  Variation (4)..(4): /replace=“u”
  Variation (11)..(11): /replace=“u”
  misc_feature (1)..(18): /note=“Variant nucleotides given in the sequence have no preference with respect to those in the annotations for variant positions”
cgctgcccaatgccaucc

SEQ ID NO: 35 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (5)..(5): /replace=“u”
  Variation (7)..(7): /replace=“u”
  Variation (12)..(12): /replace=“u”
  Variation (17)..(17): /replace=“u”
  Variation (23)..(23): /replace=“u”
  misc_feature (1)..(25): /note=“Variant nucleotides given in the sequence have no preference with respect to those in the annotations for variant positions”
gatctgtcaaatcgcctgcaggtaa

SEQ ID NO: 36 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (5)..(5): /replace=“u”
  Variation (7)..(7): /replace=“u”
  Variation (12)..(12): /replace=“u”
  Variation (17)..(17): /replace=“u”
  misc_feature (1)..(22): /note=“Variant nucleotides given in the sequence have no preference with respect to those in the annotations for variant positions”
gatctgtcaaatcgcctgcagg

SEQ ID NO: 37 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
  Variation (5)..(5): /replace=“u”
  Variation (7)..(7): /replace=“u”
  Variation (9)..(9): /replace=“u”
  Variation (14)..(14): /replace=“u”
  Variation (19)..(19): /replace=“u”
  misc_feature (1)..(24): /note=“Variant nucleotides given in the sequence have no preference with respect to those in the annotations for variant positions”
cagatctgtcaaatcgcctgcagg

SEQ ID NO: 38 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (5)..(5): /replace=“u”
  Variation (7)..(7): /replace=“u”
  Variation (12)..(12): /replace=“u”
  Variation (17)..(17): /replace=“u”
  Variation (23)..(23): /replace=“u”
  misc_feature (1)..(23): /note=“Variant nucleotides given in the sequence have no preference with respect to those in the annotations for variant positions”
gatctgtcaaatcgcctgcaggt

SEQ ID NO: 39 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (5)..(5): /replace=“u”
  Variation (7)..(7): /replace=“u”
  Variation (12)..(12): /replace=“u”
  Variation (17)..(17): /replace=“u”
  misc_feature (1)..(21): /note=“Variant nucleotides given in the sequence have no preference with respect to those in the annotations for variant positions”
gatctgtcaaatcgcctgcag

SEQ ID NO: 40 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
  Variation (5)..(5): /replace=“u”
  Variation (7)..(7): /replace=“u”
  Variation (9)..(9): /replace=“u”
  Variation (14)..(14): /replace=“u”
  Variation (19)..(19): /replace=“u”
  Variation (25)..(25): /replace=“u”
  misc_feature (1)..(25): /note=“Variant nucleotides given in the sequence have no preference with respect to those in the annotations for variant positions”
cagatctgtcaaatcgcctgcaggt

SEQ ID NO: 41 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
  Variation (5)..(5): /replace=“u”
  Variation (7)..(7): /replace=“u”
  Variation (9)..(9): /replace=“u”
  Variation (14)..(14): /replace=“u”
  Variation (19)..(19): /replace=“u”
  misc_feature (1)..(23): /note=“Variant nucleotides given in the sequence have no preference with respect to those in the annotations for variant positions”
cagatctgtcaaatcgcctgcag

SEQ ID NO: 42 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
  Variation (5)..(5): /replace=“u”
  Variation (10)..(10): /replace=“u”
  Variation (12)..(12): /replace=“u”
  Variation (15)..(16): /replace=“u”
  misc_feature (1)..(22): /note=“Variant nucleotides given in the sequence have no preference with respect to those in the annotations for variant positions”
gggatccagtatacttacaggc

SEQ ID NO: 43 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
  Variation (5)..(5): /replace=“u”
  Variation (10)..(10): /replace=“u”
  Variation (12)..(12): /replace=“u”
  Variation (15)..(16): /replace=“u”
  Variation (23)..(23): /replace=“u”
  misc_feature (1)..(25): /note=“Variant nucleotides given in the sequence have no preference with respect to those in the annotations for variant positions”
gggatccagtatacttacaggctcc

SEQ ID NO: 44 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(3): /replace=“u”
  Variation (8)..(8): /replace=“u”
  Variation (10)..(10): /replace=“u”
  Variation (13)..(14): /replace=“u”
  Variation (21)..(21): /replace=“u”
  misc_feature (1)..(23): /note=“Variant nucleotides given in the sequence have no preference with respect to those in the annotations for variant positions”
gatccagtatacttacaggctcc

SEQ ID NO: 45 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
  Variation (4)..(4): /replace=“u”
  Variation (9)..(9): /replace=“u”
  Variation (11)..(11): /replace=“u”
  Variation (14)..(15): /replace=“u”
  Variation (22)..(22): /replace=“u”
  misc_feature (1)..(24): /note=“Variant nucleotides given in the sequence have no preference with respect to those in the annotations for variant positions”
ggatccagtatacttacaggctcc

SEQ ID NO: 46 - DNA, 26 bp, Artificial Sequence
  source: /note=“Description of Artificial Sequence Synthetic oligonucleotide”
  source: /note=“Description of Combined DNA/RNA Molecule Synthetic oligonucleotide”
  Variation (3)..(4): /replace=“u”
  Variation (7)..(7): /replace=“u”
  Variation (9)..(11): /replace=“u”
  Variation (24)..(24): /replace=“u”
  misc_feature (1)..(26): /note=“Variant nucleotides given in the sequence have no preference with respect to those in the annotations for variant positions”
acttcctctttaacagaaaagcatac

SEQ ID NO: 47 - DNA, 22 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(2): /replace=“u”
  Variation (7)..(7): /replace=“u”
  Variation (9)..(9): /replace=“u”
  Variation (12)..(13): /replace=“u”
  Variation (20)..(20): /replace=“u”
  misc_feature (1)..(22): /note=“Variant nucleotides given in the sequence have no preference with respect to those in the annotations for variant positions”
atccagtatacttacaggctcc

SEQ ID NO: 48 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
  Variation (5)..(5): /replace=“u”
  Variation (10)..(10): /replace=“u”
  Variation (12)..(13): /replace=“u”
  Variation (15)..(15): /replace=“u”
  Variation (19)..(20): /replace=“u”
  Variation (23)..(23): /replace=“u”
  Variation (25)..(25): /replace=“u”
  misc_feature (1)..(25): /note=“Variant nucleotides given in the sequence have no preference with respect to those in the annotations for variant positions”
gagctcagatcttctaacttcctct

SEQ ID NO: 49 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
  Variation (5)..(5): /replace=“u”
  Variation (10)..(10): /replace=“u”
  Variation (12)..(12): /replace=“u”
  Variation (15)..(16): /replace=“u”
  Variation (23)..(23): /replace=“u”
  misc_feature (1)..(24): /note=“Variant nucleotides given in the sequence have no preference with respect to those in the annotations for variant positions”
gggatccagtatacttacaggctc

SEQ ID NO: 50 - DNA, 27 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(2): /replace=“u”
  Variation (7)..(7): /replace=“u”
  Variation (12)..(12): /replace=“u”
  Variation (14)..(14): /replace=“u”
  Variation (17)..(18): /replace=“u”
  Variation (25)..(25): /replace=“u”
  misc_feature (1)..(27): /note=“Variant nucleotides given in the sequence have no preference with respect to those in the annotations for variant positions”
atgggatccagtatacttacaggctcc

SEQ ID NO: 51 - DNA, 25 bp, Artificial Sequence
  source: /note=“Description of Artificial Sequence Synthetic oligonucleotide”
  source: /note=“Description of Combined DNA/RNA Molecule Synthetic oligonucleotide”
  Variation (2)..(2): /replace=“u”
  Variation (4)..(5): /replace=“u”
  Variation (11)..(11): /replace=“u”
  Variation (14)..(14): /replace=“u”
  Variation (18)..(19): /replace=“u”
  Variation (21)..(22): /replace=“u”
  misc_feature (1)..(25): /note=“Variant nucleotides given in the sequence have no preference with respect to those in the annotations for variant positions”
ctgttccaaatcctgcattgttgcc

SEQ ID NO: 52 - protein, 12 aa, Artificial Sequence
  source: /note=“Description of Artificial Sequence Synthetic peptide”
  MOD_RES (2)..(2): 6-aminohexanoic acid
  MOD_RES (5)..(5): 6-aminohexanoic acid
  MOD_RES (8)..(8): 6-aminohexanoic acid
  MOD_RES (11)..(11): 6-aminohexanoic acid
RXRRXRRXRRXR

SEQ ID NO: 53 - protein, 10 aa, Artificial Sequence
  source: /note=“Description of Artificial Sequence Synthetic peptide”
RFFRFFRFFR

SEQ ID NO: 54 - protein, 14 aa, Artificial Sequence
  source: /note=“Description of Artificial Sequence Synthetic peptide”
  MOD_RES (2)..(2): 6-aminohexanoic acid
  MOD_RES (5)..(5): 6-aminohexanoic acid
  MOD_RES (8)..(8): 6-aminohexanoic acid
  MOD_RES (11)..(11): 6-aminohexanoic acid
  MOD_RES (13)..(13): 6-aminohexanoic acid
  MOD_RES (14)..(14): Beta-alanine
RXRRXRRXRRXRXX

SEQ ID NO: 55 - protein, 12 aa, Artificial Sequence
  source: /note=“Description of Artificial Sequence Synthetic peptide”
  MOD_RES (11)..(11): 6-aminohexanoic acid
  MOD_RES (12)..(12): Beta-alanine
RFFRFFRFFRXX

SEQ ID NO: 56 - protein, 11 aa, Artificial Sequence
  source: /note=“Description of Artificial Sequence Synthetic peptide”
RFFRFFRFFRG

SEQ ID NO: 57 - protein, 7 aa, Artificial Sequence
  source: /note=“Description of Artificial Sequence Synthetic peptide”
RRRRRRG

SEQ ID NO: 58 - protein, 6 aa, Artificial Sequence
  source: /note=“Description of Artificial Sequence Synthetic peptide”
RRRRRR

SEQ ID NO: 59 - protein, 6 aa, Artificial Sequence
  source: /note=“Description of Artificial Sequence Synthetic peptide”
RRRRRG

SEQ ID NO: 60 - protein, 5 aa, Artificial Sequence
  source: /note=“Description of Artificial Sequence Synthetic peptide”
RRRRR

SEQ ID NO: 61 - RNA, 30 nt, Artificial Sequence
  source: /note=“Description of Artificial Sequence Synthetic oligonucleotide”
cuccaacaucaaggaagauggcauuucuag